(19) United States
(12) Patent Application Publication
(10) Pub. No.: US 2023/0293698 A1
(43) Pub. Date: Sep. 21, 2023

(54) METAL-ORGANIC FRAMEWORKS DELIVER SMALL MOLECULES AND BIOMACROMOLECULES FOR CANCER IMMUNOTHERAPY

(71) Applicant: The University of Chicago, Chicago, IL (US)

(72) Inventors: Wenbin Lin, Chicago, IL (US); Kaiyuan Ni, Chicago, IL (US); Taokun Luo, Chicago, IL (US); Guangxu Lan, Chicago, IL (US)

(21) Appl. No.: 17/927,168

(22) PCT Filed: May 24, 2021
(86) PCT No.: PCT/US2021/033886

§ 371 Date: Nov. 22, 2022

Related U.S. Application Data
(60) Provisional application No. 63/045,499, filed on Jun. 29, 2020.
(60) Provisional application No. 63/028,891, filed on May 22, 2020.

(51) Int. Cl. A61K 47/52; A61P 35/00; A61K 31/7088

(52) U.S. Cl. CPC A61K 47/52; A61P 35/00; A61K 31/7088

(57) ABSTRACT

Modified metal-organic framework (MOFs) are described that have surfaces with enhanced ability to coordinatively bond to or electrostatically interact with therapeutic agents, such as nucleic acids and small molecules and proteins with phosphate or carboxylate groups. Methods of providing the modified MOFs are described that include replacing strongly coordinating metal oxo cluster capping groups with weakly coordinating capping groups and/or incorporating organic bridging ligands with electron-withdrawing groups. MOFs with surface attached therapeutic agents (e.g., immunotherapeutic agents) prepared from the modified MOFs are also described, along with methods of using the MOFs as to treat cancer, e.g., via radiotherapy-radiodynamic therapy (RT-RDT), either with or without the co-administration of another therapeutic agent, such as a chemotherapeutic agent or an immunomodulator. Thus, the described methods can involve cancer immunotherapy and in situ cancer vaccination.

### CROSS REFERENCE TO RELATED APPLICATIONS

This application is a national stage application of PCT/US2021/033886, filed May 24, 2021, herein incorporated by reference in its entirety, and which claims priority to and the benefit of U.S. Provisional Patent Application Ser. No. 63/028,891, filed May 22, 2020; and U.S. Provisional Patent Application Ser. No. 63/045,499, filed Jun. 29, 2020, the disclosures of each of which are incorporated herein by reference in their entireties.

### GOVERNMENT INTEREST

This invention was made with government support under W81XWH-18-1-0417 awarded by the Defense Advanced Research Projects Agency, and CA198989 awarded by the National Institutes of Health. The government has certain rights in the invention.

### TECHNICAL FIELD

The presently disclosed subject matter provides compositions and methods for treating cancer. In particular, the presently disclosed subject matter relates to metal-organic framework (MOF) nanomaterials that have modified surfaces for enhanced binding to various small molecule, peptide, protein, and nucleic acid therapeutic agents. The presently disclosed subject matter further relates to the MOFs with therapeutic agent surface modification and their use in treating cancer, e.g., by activating the immune system against tumors.

### Abbreviations

° C.=degrees Celsius
%=percentage
μg=microgram
μl=microliter
mol=micromole
M=micromolar
AFM=atomic-force microscopy
APC=antigen presenting cells
APF=aminophenyl fluorescein
bpy=2,2′-bipyridine
CBI=checkpoint blockade immunotherapy
cGAMP=cyclic guanosine monophosphate-adenosine monophosphate
CLSM=confocal laser scanning microscopy
cm=centimeter
CRT=calreticulin
CTL=cytotoxic T lymphocytes
Cu=copper
DAMPs=danger-associated molecular patterns
DBB=4,4′-di(4-benzoato)-2,2′-bipyridine
DBP=5,15-di(p-benzoato)porphyrin
DC=dendritic cells
dF(CF₃)ppy=2-(2,4-difluorophenyl)-5-(trifluoromethyl) pyridine
DMF=dimethylformamide
DMSO=dimethylsulfoxide
eV=electronvolts
g=gram
Gy=gray
h=hour
Hf=hafnium
IC₅₀=fifty percent inhibitory concentration
ICD=immunogenic cell death
ICP-MS=inductively coupled plasma-mass spectrometry
IFN=interferon
IL=interleukin
IMD=imiquimod
Ir=iridium
kg=kilogram
kVp=peak kilovoltage
mA=milliampere
mg=milligram
min=minute
mL=milliliter
mm=millimeter
mM=millimolar
mmol=millimole
MOF=metal-organic framework
MOL=metal-organic layer
MOP=metal-organic nanoplates
MUC-1=mucin-1
mV=milliVolts
ng=nanogram
NIR=near infrared
nm=nanometer
nMOF=nanoscale metal-organic frameworks
NMR=nuclear magnetic resonance
ODN=oligodeoxynucleotide
PAMPs=pathogen-associated molecular patterns
PBS=phosphate buffered saline
PD-1=programming death-1
PD-L1=programming death-ligand 1
PDT=photodynamic therapy
ppy=2-phenyl-pyridine
PS=photosensitizer
Pt=platinum
PXRD=powder x-ray diffraction
RDT=radiodynamic therapy
REF₁₀=radiation enhancement factors at 10% cell survival
ROS=reactive oxygen species
RT=radiotherapy
Ru=ruthenium
SBU=secondary building units
s=seconds
SOSG=singlet oxygen sensor green
STING=stimulator of interferon genes
TEM=transmission electron microscopy
TFA=trifluoroacetic acid
TBP=5,10,15,20-tetra(p-benzoato)-porphyrin
TMS=trimethylsilyl
TGI=tumor growth inhibition
TLR9=toll-like receptor 9
TME=tumor microenvironment
UV=ultraviolet
wt=weight
Z=atomic number

### BACKGROUND

Immunotherapies for cancer harness the patient's own immune system to recognize and treat cancer. Cancer immunotherapy agents use various strategies, including checkpoint blockades, chimeric antigen receptor T cells, vaccination, and others. Checkpoint blockade immunotherapy (CBI), for example, employs agents that target T lymphocyte regulatory pathways, such as the programming death-1/programming death-ligand 1 (PD-1/PD-L1) axis. CBI has demonstrated clinical response in some solid tumors. However, many patients do not respond to current cancer immunotherapies.

Accordingly, there is an ongoing need for additional compositions and methods to treat cancer, including those that can provide cancer immunotherapy.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a metal-organic framework (MOF) having a surface modified to coordinatively or electrostatically bind to one or more therapeutic agents of interest, wherein said MOF comprises: (a) a plurality of metal oxo cluster secondary building units (SBUs), wherein each of said metal oxo cluster SBUs comprises one or more first metal ions and one or more anions, wherein each of said one or more anions is coordinated to one or more of the one or more first metal ions; and (b) a plurality of organic bridging ligands linking together the plurality of SBUs to form a two- or three-dimensional matrix; wherein (i) a plurality of SBUs at a surface of the MOF each comprise a weakly coordinating anion as a SBU capping group anion or (ii) the plurality of organic bridging ligands comprise an organic bridging ligand comprising an electron-withdrawing group or ligand, a positive charge, or a combination thereof, optionally wherein the plurality of organic bridging ligands comprise a ligand comprising a nitrogen donor group coordinatively bound to a second metal ion, wherein said second metal ion is further coordinated to at least one second metal ligand comprising one or more electron-withdrawing groups; wherein a surface of said MOF has enhanced ability to coordinatively or electrostatically bind to one or more therapeutic agents of interest.

In some embodiments, said one or more first metal ions comprise at least one ion of a metal that absorbs ionizing radiation, optionally X-rays, and/or wherein said metal is selected from the group comprising Hf, a lanthanide metal, Ba, Ta, W, Re, Os, Ir, Pt, Au, Pb, and Bi; further optionally wherein the first metal ion is a Hf ion. In some embodiments, a plurality of SBUs at a surface of the MOF each comprise a weakly coordinating anion as a capping group, optionally wherein said weakly coordinating anion is selected from the group consisting of trifluoroacetate and triflate. In some embodiments, the plurality of organic bridging ligands comprise a porphyrin substituted by at least two carboxylate groups, optionally wherein the plurality of organic bridging ligands comprise 5,15-di(p-benzoato)porphyrin (DBP).

In some embodiments, the MOF further comprises a small molecule therapeutic agent sequestered in pores and/or cavities of the two- or three-dimensional network, optionally wherein said small molecule therapeutic agent is a chemotherapeutic agent, a small molecule inhibitor and/or a small molecule immunomodulator. In some embodiments, the MOF comprises a chemotherapeutic agent sequestered in pores and/or cavities of the two- or three-dimensional network, optionally wherein said chemotherapeutic agent is selected from cisplatin, carboplatin, paclitaxel, SN-35, and etoposide. In some embodiments, the MOF comprises a small molecule inhibitor sequestered in pores and/or cavities of the two- or three-dimensional network, optionally wherein said small molecule inhibitor is selected from the group consisting of a PLK1 inhibitor, a Wnt inhibitor, a Bcl-2 inhibitor, a PD-L1 inhibitor, an ENPP1 inhibitor and an IDO inhibitor. In some embodiments, the MOF comprises a small molecule immunomodulator sequestered in pores and/or cavities of the two- or three-dimensional network. In some embodiments, the small molecule immunomodulator is imiquimod (IMD).

In some embodiments, the plurality of organic bridging ligands comprise an organic bridging ligand comprising a nitrogen donor group, wherein said nitrogen donor group is coordinated to a second metal ion and wherein said second metal ion is further coordinated to at least one second metal ligand comprising one or more electron-withdrawing groups, optionally wherein the one or more electron withdrawing groups are selected from halo and perhaloalkyl groups. In some embodiments, the organic bridging ligand comprising a nitrogen donor group is 4,4′-di(p-benzoato)-2,2′-bipyridine (DBB). In some embodiments, the second metal ion is an iridium (Ir) ion or a ruthenium (Ru) ion and/or wherein said second metal ion is coordinated to two second metal ligands, wherein one or both of the second metal ligands comprise one or more electron withdrawing groups. In some embodiments, one or both of the second metal ligands is 2-(2,4-difluorophenyl)-5-(trifluomethyl)pyridine (dF(CF₃)ppy). In some embodiments, the MOF has a zeta (ζ)-potential value of at least about 5 millivolts (mV), optionally wherein the MOF has a ζ-potential value of at least about 30 mV.

In some embodiments, said MOF comprises a three-dimensional network, wherein said three-dimensional network is provided in the form of a nanoparticle.

In some embodiments, the presently disclosed subject matter provides a MOF for the delivery of one or more therapeutic agents of interest, wherein said MOF comprises: (a) a plurality of metal oxo cluster SBUs, wherein each of said metal oxo cluster SBUs comprises one or more first metal ions and one or more anions, wherein each of said anions is coordinated to one or more of the one or more first metal ions; (b) a plurality of organic bridging ligands linking together the plurality of SBUs to form a two- or three-dimensional matrix; and (c) one or more therapeutic agents of interest bonded to a surface of said MOF via coordinative bonds or electrostatic interactions, optionally wherein one or more therapeutic agents of interest are coordinatively bonded to a metal ion of one or more of the plurality of SBUs at the surface of the MOF. In some embodiments, said first metal ion is an ion of a metal that absorbs ionizing radiation, optionally X-rays, and/or wherein the first metal ion is an ion of a metal selected from Hf, a lanthanide metal, Ba, Ta, W, Re, Os, Ir, Pt, Au, Pb, and Bi; further optionally wherein the first metal ion is a Hf ion.

In some embodiments, each of said one or more therapeutic agents of interest are selected from the group comprising a nucleic acid, a small molecule comprising a phosphate or carboxylate group, and/or a macromolecule comprising a surface accessible phosphate or carboxylate group. In some embodiments, the one or more therapeutic agents of interest comprise a macromolecule comprising a surface accessible phosphate or carboxylate group and wherein said macromolecule is a protein, optionally wherein said protein is an antibody. In some embodiments, said protein is selected from the group comprising an anti-CD37 antibody, an anti-CD44 antibody, an anti-CD47 antibody, an anti-CD73 antibody, an anti-PD-1 antibody, an anti-PD-L1 antibody, an anti-LAG3 antibody, and an anti-CTLA-4 antibody. In some embodiments, the one or more therapeutic agents of interest comprise a nucleic acid and wherein said nucleic acid is selected from the group comprising a miRNA, a mRNA, a siRNA, a CpG ODN, and a cyclic di-nucleotide, optionally wherein the nucleic acid is a cyclic di-nucleotide and said cyclic di-nucleotide is a STING agonist, further optionally wherein said STING agonist is c-di-AMP or cGAMP.

In some embodiments, said MOF comprises one or more additional therapeutic agents sequestered in pores or cavities of the two- or three-dimensional network; optionally wherein said MOF comprises about 1 wt % to about 50 wt % of said one or more additional therapeutic agents.

In some embodiments, the plurality of SBUs comprise Hf oxo clusters, wherein said plurality of organic bridging ligands comprise DBP, and wherein the one or more therapeutic agents of interest are bonded to the surface of said MOF via coordinative bonds to Hf ions of surface accessible SBUs. In some embodiments, the one or more therapeutic agents of interest comprise one or more antibodies. In some embodiments, the one or more therapeutic agents comprise an anti-CD47 antibody. In some embodiments, the MOF further comprises IMD sequestered in pores or cavities of the two- or three-dimensional network.

In some embodiments, said MOF is a three-dimensional network and is provided as a nanoparticle. In some embodiments, said MOF comprises about 1 wt % to about 50 wt % of the IMD or the anti-CD47 antibody; optionally wherein the MOF comprises about 9 weight (wt) % IMD and about 7.5 wt % anti-CD47 antibody.

In some embodiments, the plurality of SBUs comprise Hf oxo clusters, wherein said plurality of organic bridging ligands comprise DBB coordinated to an Ir ion, wherein said Ir ion is further coordinated to two (dF(CF₃)ppy); and wherein the one or more therapeutic agents of interest are bonded to the surface of said MOF via electrostatic interactions. In some embodiments, the one or more therapeutic agents of interest comprise a nucleic acid. In some embodiments, the nucleic acid is a STING agonist or a CpG oligodeoxynucleotide (ODN), optionally wherein the nucleic acid is a CpG ODN.

In some embodiments, the MOF comprises about 1 wt % to about 50 wt % of the one or more therapeutic agents of interest, optionally wherein said one or more therapeutic agents of interest comprise an antibody.

In some embodiments, the presently disclosed subject matter provides a method of treating cancer in need thereof, the method comprising: (a) administering to the subject a MOF, wherein said MOF comprises: (i) a plurality of metal oxo cluster SBUs, wherein each of said metal oxo cluster SBUs comprises one or more first metal ions and one or more anions, wherein each of said anions is coordinated to one or more of the one or more first metal ions; (ii) a plurality of organic bridging ligands linking together the plurality of SBUs to form a two- or three-dimensional matrix; and (iii) one or more therapeutic agents of interest bonded to a surface of said MOF via coordinative bonds or electrostatic interactions, optionally wherein one or more therapeutic agents of interest are coordinatively bonded to a metal ion of one or more of the plurality of SBUs at the surface of the MOF; and (b) exposing at least a portion of the subject to ionizing radiation energy, optionally X-rays. In some embodiments, the method further comprises administering to said subject an additional therapeutic agent or treatment, optionally an immunotherapy agent and/or a cancer treatment selected from the group comprising surgery, chemotherapy, toxin therapy, cryotherapy, and gene therapy.

In some embodiments, the additional therapeutic agent is an immunotherapy agent, optionally wherein said immunotherapy agent is an immune checkpoint inhibitor. In some embodiments, the immunotherapy agent is an anti-PD-1 or an anti-PD-L1 antibody.

In some embodiments, the cancer is colorectal cancer, melanoma, head and neck cancer, brain cancer, breast cancer, liver cancer, cervical cancer, lung cancer or pancreatic cancer. In some embodiments, administration of the MOF provides an extended release profile for one or more of the one or more therapeutic agents of interest, optionally wherein the release rate is tunable and/or wherein the MOF provides sustained release of one or more therapeutic agents of interest over a period of a few hours or a few days. In some embodiments, administration of the MOF lowers the therapeutically effective dose of the one or more therapeutic agents of interest.

In some embodiments, the presently disclosed subject matter provides a method of enhancing surface interaction and/or bonding of one or more therapeutic agents of interest to a MOF, the method comprising modifying the surface of the MOF by (i) providing one or more surface accessible coordination sites coordinatively bonded to a weakly coordinated anion that can be replaced by a carboxylate or phosphate substituent of a therapeutic agent of interest or (ii) providing a MOF comprising one or more electron-withdrawing bridging ligands, one or more bridging ligands comprising a positive charge, or a combination thereof. In some embodiments, modifying the surface of the MOF comprises: (ia) providing a parent MOF comprising metal oxo cluster SBUs linked together via organic bridging ligands, wherein each of said SBUs comprises one or more metal ions and one or more anions, and wherein said MOF comprises a plurality of surface accessible metal oxo cluster SBUs where the one or more anions of each of said surface accessible metal oxo cluster SBUs comprise a strongly coordinating anion as a SBU capping group; optionally wherein said strongly coordinating anion comprises acetate or formate; and (ib) removing said strongly coordinating anion, wherein the removing comprises contacting said parent MOF with a reagent selected from trimethylsilyl trifluoroacetate, trimethylsilyl triflate, and a mineral acid having a pKa of less than about 3; thereby replacing said strongly coordinating anion, optionally wherein said strongly coordinating anion is selected from an acetate or a formate anion, with a weakly coordinating anion, optionally wherein said weakly coordinating anion is selected from a trifluoroacetate or triflate anion.

In some embodiments, providing a MOF comprising one or more bridging ligands comprising an electron-withdrawing group, one or more bridging ligands comprising a positive charge, or a combination thereof comprises providing a MOF comprising metal oxo cluster SBUs linked together via organic bridging ligands, wherein each of said SBUs comprise one or more first metal ions and one or more anions coordinated to said one or more first metal ions, and wherein said organic bridging ligands comprise at least one organic bridging ligand comprising a coordinated, non-SBU-associated second metal ion, wherein said second metal ion is further coordinated to one or more electron-withdrawing ligand, optionally wherein said electron-withdrawing ligand is a halo and/or perhaloalkyl-substituted bipyridine ligand. In some embodiments, providing the MOF comprises providing an MOF comprising a DBB bridging ligand, wherein said DBB bridging ligand is coordinated to a first metal ion of two different metal oxo cluster SBUs and to a second metal ion and wherein said second metal ion is further coordinated to two halo and/or perhaloalkyl-substituted pyridine ligands, optionally wherein said two halo and/or perhaloalkyl-substituted pyridine ligands are each 2-(2,4-difluorophenyl)-5-(trifluoromethyl)-pyridine. In some embodiments, said second metal ion is Ir or Ru.

In some embodiments, the MOF comprises one or more SBU comprising a metal ion that absorbs ionizing radiation, optionally x-rays and/or wherein the metal ion is an ion of an element selected from the group comprising Hf, a lanthanide metal, Ba, Ta, W, Re, Os, Ir, Pt, Au, Pb, and Bi; further optionally wherein said metal ion is a Hf ion. In some embodiments, said MOF has enhanced interaction and/or bonding ability for one or more therapeutic agents of interest compared to a MOF without surface modification, wherein said one or more therapeutic agents of interest are selected from a nucleic acid, a small molecule, and/or macromolecule comprising a surface accessible phosphate or carboxylate group. In some embodiments, said protein is selected from the group comprising an anti-CD37 antibody, an anti-CD44 antibody, an anti-CD47 antibody, an anti-CD73 antibody, an anti-PD-1 antibody, an anti-PD-L1 antibody, an anti-LAG3 antibody, and an anti-CTLA-4 antibody. In some embodiments, said nucleic acid is selected from the group comprising a miRNA, a mRNA, a siRNA, a CpG ODN, and a cyclic di-nucleotide, optionally wherein said cyclic di-nucleotide is a STING agonist, further optionally wherein said STING agonist is c-di-AMP or cGAMP.

Accordingly, it is an object of the presently disclosed subject matter to provide MOFs adapted to bind and deliver one or more therapeutic agents of interest, to methods of treating cancer using the MOFs, and to methods of enhancing surface interaction and/or binding of therapeutic agents to MOFs.

An object of the presently disclosed subject matter having been stated hereinabove, and which is achieved in whole or in part by the presently disclosed subject matter, other objects will become evident as the description proceeds when taken in connection with the accompanying drawings and examples as best described hereinbelow.

### BRIEF DESCRIPTION OF THE DRAWINGS

FIG. 1: A schematic drawing showing repolarization of M2 to M1 macrophages and promotion of phagocytosis by blocking the “don't eat me” signal on tumor cells by a metal-organic framework (MOF) comprising hafnium (Hf)-oxo clusters and 5,15-di(p-benzoato)porphyrin (DBP) bridging ligands with imiquimod (IMD) sequestered in pores in the MOF and anti-cluster of differentiation 47 (αCD47) antibodies attached to the surface of the MOF (wherein the MOF is referred to as IMD@Hf-DBP/αCD47) plus X-ray radiation. This macrophage therapy synergizes with anti-programming death-ligand 1 antibody (αPD-L1) checkpoint blockade immunotherapy (CBI) to systemically eradicate tumors.

FIGS. 2A-2C: FIG. 2A is a schematic drawing showing surface modification of metal-oxo clusters of hafnium-5,15-di(p-benzoato)porphyrin (Hf-DBP) metal-organic frameworks (MOFs) for anti-cluster of differentiation 47 antibody (αCD47) loading. A parent Hf-DBP with acetate capping groups on the hafnium (Hf) oxo clusters (left) is contacted with trimethylsilyl-trifluoroacetate (TMS-TFA), exchanging the acetate capping groups with trifluoroacetate capping groups (middle) which are then replaced by αCD47. FIG. 2B is a graph showing the αCD47 loading efficiency of Hf-DBP (right) and TFA-modified Hf-DBP (left). FIG. 2C is a graph showing the release profiles of imiquimod (IMD, squares)) and αCD47 (circles) from an Hf-DBP MOF with IMD sequestered in MOF pores and αCD47 attached to the surface (i.e., where the MOF is IMD@Hf-DBP/αCD47).

FIGS. 3A and 3B: Transmission electron microscopy (TEM) images of hafnium-5,15-di(p-benzoato)porphyrin (Hf-DBP) metal-organic frameworks (MOFs). FIG. 3A is a large field TEM image. Scale bar at lower left is 200 nanometers (nm). FIG. 3B is a high resolution TEM image (scale bar at lower left is 20 nm) with a cropped fast Fourier transfer (FFT) image (inset, lower right).

FIGS. 4A and 4B: Transmission electron microscopy (TEM) images of trifluoroacetate (TFA)-modified hafnium-5,15-di(p-benzoato)porphyrin (Hf-DBP) metal-organic frameworks (MOFs). FIG. 4A is a large field TEM image. Scale bar at lower left is 200 nanometers (nm). FIG. 4B is a high resolution TEM image (scale bar at lower left is 10 nm) with a cropped fast Fourier transfer (FFT) image (inset, lower right).

FIG. 5: Graph showing the powder x-ray diffraction (PXRD) patterns of a hafnium-5,15-di(p-benzoato)porphyrin (Hf-DBP, solid line) metal-organic framework (MOF) and of a trifluoroacetate (TFA)-modified Hf-DBP MOF (dashed line).

FIG. 6: A schematic drawing showing imiquimod (IMD) loaded in a trifluoroacetate (TFA)-modified hafnium-5,15-di(p-benzoato)porphyrin) (Hf-DBP) nanopalate. Hafnium-12 (Hf₁₂) secondary binding units (SBUs) are shown as polyhedra, DBP organic ligands are shown as sticks. The trifluoromethyl moieties of the TFA groups and IMD are indicated by arrows.

FIGS. 7A and 7B: Transmission electron microscopy (TEM) images of IMD@Hf-DBP, i.e., imiquimod (IMD)-loaded hafnium-5,15-di(p-benzoato)porphyrin (Hf-DBP) metal-organic frameworks (MOFs). FIG. 7A is a large field TEM image. Scale bar at lower left is 200 nanometers (nm). FIG. 7B is a high resolution TEM image (scale bar at lower left is 50 nm) with a cropped fast Fourier transfer (FFT) image (inset, lower right).

FIGS. 8A and 8B: Transmission electron microscopy (TEM) images of IMD@Hf-DBP/αCD47, i.e., imiquimod (IMD)-loaded hafnium-5,15-di(p-benzoato)porphyrin (Hf-DBP) metal-organic frameworks (MOFs) with anti-cluster of differentiation 47 antibodies (αCD47) attached to the surface. FIG. 8A is a large field TEM image. Scale bar at lower left is 100 nanometers (nm). FIG. 8B is a high resolution TEM image (scale bar at lower left is 50 nm) with a cropped fast Fourier transfer (FFT) image (inset, lower right).

FIG. 9: Graph showing the powder x-ray diffraction (PXRD) patterns of a hafnium-5,15-di(p-benzoato)porphyrin (Hf-DBP, solid line) metal-organic framework (MOFs) loaded with imiquimod (IMD), i.e., IMD@Hf-DBP (solid line) and of a Hf-DBP MOF loaded with IMD and having anti-cluster of differentiation 47 antibodies (αCD47) attached to the MOF surface, i.e., IMD@Hf-DBP/αCD47 (dashed line).

FIG. 10: Graph showing the release (percentage versus time (in hours (hr))) of fluorescein-isothiocyanate (FITC)-labeled immunoglobulin G (IgG-FITC) from an imiquimod (IMD)-loaded hafnium-5,15-di(p-benzoato)porphyrin metal organic framework surface modified with the IgG-FITC (i.e., IMD@Hf-DBP/IgG-FITC) in serum containing phosphate buffered saline (PBS). n=3.

FIG. 11: Graph showing the cellular uptake (measured as nanomoles of hafnium (nmol Hf) per 10⁵ cells) of hafnium-5,15-di(p-benzoato)porphyrin (Hf-DBP) metal-organic frameworks (MOFs) in murine colon adenocarcinoma (CT26) cells as quantified by inductively-coupled plasma-mass spectrometry (ICP-MS). Cellular uptake is shown for imiquimod (IMD) loaded Hf-DBP (IMD@Hf-DBP, darkly shaded bars) and for IMD-loaded Hf-DBP with surface attached anti-cluster of differentiation 47 antibodies (αCD47) (IMD@Hf-DBP/αCD47, light grey shaded bars). n=3.

FIG. 12: Graph showing the dark toxicity (no X-ray irradiation) of hafnium-5,15-di(p-benzoato)-porphyrin (Hf-DBP) metal organic frameworks (MOFs) loaded with imiquimod (IMD) (i.e., IMD@Hf-DBP, circles) and IMD@Hf-DBP MOFs with surface attached anti-cluster of differentiation 47 antibodies (IMD@Hf-DBP/αCD47, squares) in murine colon adenocarcinoma (CT26) cells. Cell viability (shown as a percentage (%)) was measured by (3-(4,5-dimethylthiazol-2-yl)-5-(3-carboxymethoxyphenyl)-2-(4-sulfophenyl)-2H-tetrazolium) (MTS) assay. n=6.

FIG. 13: Representative gating strategies for macrophages and their subtypes M1 and M2. Histograms indicate different expression levels of cluster of differentiation 86 (CD86) or cluster of differentiation 206 (CD206) in M1 or M2 subtypes, respectively.

FIGS. 14A-14D: FIG. 14A is a series of representative flow cytometric analysis of repolarization of M2 macrophages co-cultured with murine colon adenocarcinoma (CT26) cells treated with phosphate buffered saline (PBS), imiquimod (IMD), hafnium-5,15-di(p-benzoato)porphyrin (Hf-DBP) metal-organic frameworks (MOFs) or IMD-loaded Hf-DBP (IMD@Hf-DBP) MOFs and irradiated with X-ray at a dose of 0 (−) or 2 (+) Gray (Gy). Macrophages were stained with phycoerythrin-cyanine dye (PE-Cy7)-conjugated cluster of differentiation 206 (CD206) and allophycocyanin (APC)-conjugated cluster of differentiation 86 (CD86) antibodies. FIG. 14B is a series of representative flow cytometric analysis of phagocytosis of carboxyfluorescein succinimidyl ester (CFSE)-labeled CT26 cells by macrophages treated with PBS, anti-cluster of differentiation 47 antibody (αCD47), Hf-DBP or αCD47 surface modified Hf-DBP (Hf-DBP/αCD47) and irradiated with X-ray at a dose of 0 (−) or 2(+) Gray (Gy). CFSE-labeled murine colon adenocarcinoma (CT26) cells were gated from peridinin-chlorophyll protein-cyanine dye 5.5 (PerCP-Cy5.5)-labeled macrophage populations. FIG. 14C is a graph showing the quantification of macrophage repolarization described for FIG. 14A. FIG. 14D is a graph showing the quantification of phagocytosis described for FIG. 14B. n=3; *P<0.05, **P<0.01, and ***P<0.005 from control.

FIGS. 15A-15C: FIG. 15A is a series of histograms showing the immune analysis of macrophage repolarization in murine colon adenocarcinoma (CT26)-tumors treated with phosphate buffered saline (PBS) and no X-ray irradiation (PBS(−)); PBS with irradiation (PBS(+)); a mixture of imiquimod (IMD) and anti-cluster of differentiation 47 antibody (αCD47) with irradiation (IMD/αCD47(+)); IMD-loaded hafnium-5,15-di(p-benzoate)porphyrin metal-organic framework (Hf-DBP) with surface attached αCD47 and no irradiation (IMD@Hf-DBP-αCD47(−)); Hf-DBP and irradiation (Hf-DBP(+)); IMD-loaded Hf-DBP and irradiation (IMD@Hf-DBP(+)); Hf-DBP with surface attached αCD47 and irradiation (Hf-DBP-αCD47(+)); or IMD@Hf-DBP-αCD47 and irradiation (IMD@Hf-DBP-αCD47(+)). FIG. 15B is a graph showing the mean fluorescence intensity (MFI) of major histocompatibility complex class II (MHC-II) expression in the same tumor treatment groups described for FIG. 15A. FIG. 15C is a graph showing the efficacy curves (indicated by tumor volume in cubic centimeters (cm³)) of the various treatments described for FIG. 15A. n=5 or 6.

FIG. 16: Representative gating strategies for cluster of differentiation 45 positive (CD45+) cells, cluster of differentiation 11b positive (CD11b+) cells, dendritic cells (DCs), macrophages and their subtypes M1 and M2.

FIGS. 17A-17F: FIG. 17A is a graph showing the percentages of cluster of differentiation 45 positive (CD45+) cells in the murine colon adenocarcinoma (CT26) tumor-bearing mice treated as described for FIG. 15A. FIG. 17B is a graph showing the percentages of dendritic cells (DCs) in the murine colon adenocarcinoma (CT26) tumor-bearing mice treated as described for FIG. 15A. FIG. 17C is a graph showing the percentages of macrophages in the murine colon adenocarcinoma (CT26) tumor-bearing mice treated as described for FIG. 15A. FIG. 17D is a graph showing the percentages of M1 macrophages in the murine colon adenocarcinoma (CT26) tumor-bearing mice treated as described for FIG. 15A. FIG. 17E is a graph showing the percentages of M2 macrophages in the murine colon adenocarcinoma (CT26) tumor-bearing mice treated as described for FIG. 15A. FIG. 17F is a graphs showing the ratio of M1 to M2 macrophages in the murine colon adenocarcinoma (CT26) tumor-bearing mice treated as described for FIG. 15A. (+) and (−) refer to with and without X-ray irradiation, respectively. Central lines, bounds of boxes, and whiskers represent mean values, 25% to 75% of the range of data, and 1.5 fold of interquartile range away from outliers, respectively. *P<0.05, **P<0.01 and ***P<0.001 by t-test.

FIG. 18: Photo of excised tumors from murine colon adenocarcinoma (CT26) tumor-bearing mice treated with phosphate buffered saline (PBS) and no X-ray irradiation (PBS(−)); PBS with irradiation (PBS(+)); a mixture of imiquimod (IMD) and anti-cluster of differentiation 47 antibody (αCD47) with irradiation (IMD/αCD47(+)); IMD-loaded hafnium-5,15-di(p-benzoate)porphyrin metal-organic framework (Hf-DBP) with surface attached αCD47 and no irradiation (IMD@Hf-DBP-αCD47(−)); Hf-DBP and irradiation (Hf-DBP(+)); IMD-loaded Hf-DBP and irradiation (IMD@Hf-DBP(+)); Hf-DBP with surface attached αCD47 and irradiation (Hf-DBP-αCD47(+)); or IMD@Hf-DBP-αCD47 and irradiation (IMD@Hf-DBP-αCD47(+)) in a CT26 single tumor model after sacrifice. n=6.

FIG. 19: Graph of tumor weights (in grams (g)) from each treatment group described for FIG. 18 in the murine colon adenocarcinoma tumor model after sacrifice. N=6.

FIGS. 20A-20F: FIG. 20A is a graph of the growth curves of primary tumors of bilateral murine colon adenocarcinoma (CT26) tumor-bearing mice treated with phosphate buffered saline (PBS) and no irradiation (PBS(−)); PBS with X-ray irradiation (PBS(+)); anti-programming death ligand 1 antibody ((αPD-L1) and irradiation (αPD-L1(+)); imiquimod (IMD) loaded hafnium-5,15-di(p-benzoato)porphyrin (Hf-DBP) metal-organic frameworks (MOFs) with surface attached anti-cluster of differentiation 47 antibodies ((αCD47) and irradiation (IMD@Hf-DBP/αCD47(+)); Hf-DBP MOFs with surface attached αCD47 antibodies, irradiation and co-administered αPD-L1 (Hf-DBP/αCD47(+)+αPDL1); IMD, αCD47, αPDL1 and irradiation (IMD/αCD47(+)+αPDL1); IMD-loaded Hf-DBP MOFs with surface attached αCD47, no irradiation and co-administered αPDL1 (IMD@Hf-DBP/αCD47(−)+αPD-L1); or IMD loaded Hf-DBP MOFs with surface attached αCD47 with irradiation and co-administration of αPD-L1 (IMD@Hf-DBP/αCD47(+)+αPD-L1). FIG. 20B is a graph of the growth curves of distant tumors in the mice described for FIG. 20A. FIG. 20C is a graph of ELISpot assay results to detect interferon-gamma (IFN-γ) producing T cells with tumor-specific responses in splenocytes after six of the eight treatments described for FIG. 20A. Results are shown for treatment of cells stimulated with no peptide (left) or with SPSYVYHQF (SEQ ID NO: 4) (right). FIGS. 20D-20F are graphs of the percentages of tumor-infiltrating cluster of differentiation 8 positive (CD8⁺, FIG. 20D), cluster of differentiation 4 positive (CD4⁺) T cells (FIG. 20E), and natural (NK) cells (FIG. 20F) with respect to the total number of tumor cells for six of the eight treatment groups described for FIG. 20A. n=5. *P<0.05, **P<0.01, and ***P<0.005 from control.

FIG. 21 is a series of histograms showing representative gating strategies for cluster of differentiation 45 positive (CD45⁺) cells, T cells, cluster of differentiation 8 positive (CD8⁺) T cells, B cells, natural killer (NK) cells, dendritic cells, macrophages and their subtypes of M1 and M2.

FIGS. 22A and 22B: A pair of graphs of the percentages of cluster of differentiation 45 positive (CD45⁺) cells (FIG. 22A) and B cells (FIG. 22B) in both primary and distant tumors of murine colon adenocarcinoma (CT26) bilateral tumor bearing mice treated as described for six of the eight treatment groups in FIG. 20A. (+) and (−) refer to with and without irradiation, respectively. Central lines, bounds of boxes, and whiskers represent mean values, 25% to 75% of the range of data, and 1.5 fold of interquartile range away from outliers, respectively. *P<0.05, **P<0.01 and ***P<0.001 by t-test.

FIG. 23 is a schematic illustration of antitumor effect of in situ cancer vaccination via nanoscale metal-organic frameworks (nMOFs) plus checkpoint blockade immunotherapy (CBI). (1) Hf-DBB^{F}-Ir@CpG (i.e., a MOF with hafnium secondary building units; organic bridging ligands comprising a 4,4′-di(4-benzoato)-2,2′-bipyridine (DBB) coordinatively bound to an iridium (IR) also coordinatively bound to two 2-(2,4-difluorophenyl)-5-trifluoromethyl)pyridine ligands; and surface bound CpG oligodeoxynucleotides (ODNs)) was intratumorally administrated in the primary tumor. (2) Upon X-ray activation, Hf-DBB^{F}-Ir generate reactive oxygen species (ROSs) to induce immunogenic cell death to expose tumor antigens and danger-associated molecular patterns (DAMPs), while CpG ODNs as pathogen-associated molecular patterns (PAMPs) deliver to antigen presenting cells assisted by cationic Hf-DBB^{F}_Ir. (3) DAMPs and PAMPs promote dendritic cell (DC) maturation. (4) Tumor antigens are presented by mature DCs onto T cell in tumor-draining lymph nodes. (5) T cells are expanded and primed to the distant tumor as well as the primary tumor. (6) Systemically administrated immune checkpoint blockade inhibitor anti-programming death ligand 1 antibodies (αPD-L1) attenuates T cell exhaustion.

FIGS. 24A and 24B are a pair of schematic diagrams showing synthesis of Hf-DBB^{F}-Ir (FIG. 24A) and Hf-DBB-Ir (FIG. 24B) metal-organic frameworks (MOFs) from hafnium tetrachloride (HfCl₄) and either H₂DBB-Ir-F (FIG. 24A) or H₂DBB-Ir (FIG. 24B).

FIGS. 25A and 25B: FIG. 25A is a graph showing the powder x-ray diffraction patterns (PXRDs) of Hf-DBB^{F}-Ir and Hf-DBB-Ir, freshly prepared or after 24 h incubation in 0.6 mM phosphate buffered saline (PBS), in comparison to that of a model metal-organic framework (University of Oslo (UiO)-69). FIG. 25B is a graph showing the number-averaged diameters of Hf-DBB^{F}-Ir (112.2±2.8 nanometers (nm), squares) and Hf-DBB-Ir (113.9±1.6 nm, circles) in ethanol (EtOH). n=3.

FIGS. 26A-26E: FIG. 26A is a schematic illustration of controlled synthesis of Hf-DBB-Ir and Hf-DBB^{F}-Ir nMOFs based on hafnium (Hf)-oxo clusters and DBB^{F}_Ir or DBB-Ir ligands, respectively. Upon X-ray irradiation, Hf-oxo clusters absorb X-ray to generate ^{·}OH through radiolysis and transfer energy to adjacent photosensitizing ligands to generate ¹O₂ and/or O₂. FIG. 26B is a pair of transmission electron microscopy (TEM) images of Hf-DBB-Ir (top) and Hf-DBB^{F}-Ir (bottom), Scale bar=100 nm. FIGS. 26C-26E are graphs showing ^{·}OH generation probed by aminophenyl fluorescein (APF) with a fluorescence imaging system (FIG. 26C, n=6), 102 generation probed by singlet oxygen sensor green (SOSG) with a fluorescence imaging system (FIG. 26D, n=6), and 02 generation probed by 5-tert-butoxycarbonyl-5-methyl-1-pyrroline-N-oxide (BMPO) in electron paramagnetic resonance (EPR) (FIG. 26E) of DBB-Ir, DBB^{F}-Ir, Hf-DBB-Ir, or Hf-DBB^{F}-Ir upon X-ray irradiation.

FIGS. 27A-27D: Transmission electron microscopy (TEM) images of Hf-DBB^{F}_Ir and Hf-DBB-Ir metal organic frameworks (MOFs), with and without surface-attached CpG oligodeoxynucleotides (ODNs). FIGS. 27A and 27B are large-area TEM images of Hf-DBB^{F}-Ir and Hf-DBB-Ir, respectively. Scale bar in the lower right of both images represents 500 nanometers (nm). FIGS. 27C and 27D are TEM images of Hf-DBB^{F}_Ir@CpG. In FIG. 27C, the scale bar in the lower right represents 100 nm, while the scale bar in the lower right of FIG. 27D represents 500 nm.

FIGS. 28A-28D: FIG. 28A is a graph showing the ultraviolet-visible (UV-vis) spectra (intensity (in arbitrary units) versus wavelength in nanometers (nm)) of Hf-DBB^{F}-Ir and Hf-DBB-Ir metal-organic frameworks (MOFs) in comparison to those of ligands DBB^{F}-Ir and DBB-Ir. FIGS. 28B and 28C are graphs of the excitation (Ex) and emission (Em) spectra of Hf-DBB^{F}-Ir (FIG. 28B) and Hf-DBB-Ir (FIG. 28C) in comparison to those of DBB^{F}-Ir and DBB-Ir, respectively. FIG. 28D is a graph comparing the excitation (Ex) and emission (Em) spectra of Hf-DBB^{F}-Ir and Hf-DBB-Ir.

FIG. 29: Graph of cellular uptake of Hf-DBB-Ir and Hf-DBB^{F}-Ir in murine colon carcinoma (MC38) cells after 1, 2, 4, or 8 hours of incubation with 20 micromolar (M) Hf-DBB-Ir or Hf-DBB^{F}-Ir based on Hf, n=3. The hafnium (Hf) concentrations (in nanomoles (nmol) per 10% cells) were determined by inductively-coupled plasma mass spectrometry (ICP-MS).

FIGS. 30A-30D: In vitro generation of danger-associated molecular patterns (DAMPs) and phagocytosis. Murine colon carcinoma (MC38) cells were treated with phosphate buffered saline (PBS), ligands (DBB-Ir or DBB^{F}-Ir) or metal-organic frameworks (MOFs) (Hf-DBB-Ir or Hf-DBB^{F}-Ir) for 4 hours and then irradiated with X-rays at a dose of 0 (−) or 2 (+) Gray (Gy). FIG. 30A is a graph of results of clonogenic assays to evaluate radioenhancement of Hf-DBB-Ir or Hf-DBB^{F}-Ir on MC38 cells upon X-ray irradiation, n=6. FIG. 30B is a graph of the cytotoxicity of Hf-DBB-Ir or Hf-DBB^{F}-Ir upon X-ray irradiation at a dose of 2 Gy on MC38 cells, n=6. FIGS. 30C and 30D are graphs of phagocytosis of carboxyfluorescein succinimidyl ester (CFSE)-labelled MC38 cells by dendritic cells (DCs). DCs co-cultured with treated MC38 cells without (−, FIG. 30C) or with (+, FIG. 30D) irradiation were stained with phycoerythrin-cyanine dye 5.5 (PE-Cy5.5)-conjugated cluster of differentiation 11c (CD11c) antibody, n=3. Data are expressed as means±s.d. *P<0.05, **P<0.01 and ***P<0.001 by t-test.

FIGS. 31A and 31B: Generation of danger-associated molecular patterns (DAMPs) and phagocytosis. Murine colon carcinoma (MC38) cells were treated with PBS, DBB-Ir, DBB^{F}-Ir, Hf-DBB-Ir, or Hf-DBB^{F}-Ir for 4 hours and then irradiated upon X-ray at a dose of 0 (−) or 2 (+) Gray (Gy) and co-cultured with dendritic cells (DCs) for phagocytosis assay. FIG. 31A is a series of graphs showing the flow cytometric analysis of calreticulin exposure in treated MC38 cells. Lighter grey histogram (control) and darker grey histogram show the difference of calreticulin (CRT) level in the cells, respectively. FIG. 31B is a series of graphs showing phagocytosis of carboxyfluorescein succinimidyl ester (CFSE)-labelled MC38 cells by DCs analyzed by flow cytometry. DCs co-cultured with treated MC38 cells were stained with phycoerythrin-cyanine dye 5.5 (PE-Cy5.5)-conjugated CD11c antibody. CD11c⁺CFSE⁺ double positive population was gated as DC-phagocytosed MC38 cells.

FIGS. 32A-32I: In vitro delivery of pathogen-associated molecular patterns (PAMPs) and dendritic cell (DC) activation. FIG. 32A is a graph showing the zeta (C) potential (in millivolts (mV)) of Hf-DBB-Ir and Hf-DBB^{F}-Ir metal-organic frameworks (MOFs). FIG. 32B is a graph showing (left lanes) quantification of adsorbed CpG oligodeoxynucleotides (ODNs) with DNA gel (insert) and (right lanes) non-absorbed CpG ODN by NanoDrop with free CpG ODN as control. FIG. 32C is a series of graphs and fluorescence micrograph images of fluorescein isothiocyanate (FITC)-labeled CpG ODN uptake by DCs incubated with free CpG ODNs, Hf-DBB-Ir@CpG or Hf-DBB^{F}-Ir@CpG at concentrations of 20 micromolar (M) hafnium (Hf) and 0.1 microgram per milliliter (g/mL) CpG ODNs quantified with flow cytometry and observed under confocal laser scanning microscopy (CLSM). Scale bar=4 micrometers (m). FIGS. 32D-32F are graphs showing quantification of functional surface markers cluster of differentiation 80 (CD80, FIG. 32D), cluster of differentiation 86 (CD86, FIG. 32E) and major histocompatibility complex class II (MHC-II, FIG. 32F) quantified by flow cytometry. The legend in FIG. 32D applies to all three of FIGS. 32D-32F. FIGS. 32G and 32H are graphs of quantification of biomarkers interferon-alpha (IFN-α, FIG. 32G) and interleukin-6 (IL-6, FIG. 32H) of DCs stimulated with free CpG ODNs, Hf-DBB-Ir@CpG or Hf-DBB^{F}-Ir@CpG with increasing CpG ODNs concentrations by enzyme-linked immunosorbent assay (ELISA), n=6. FIG. 32I is a graph of the ovalbumin Kb-binding protein (SIINFEKL (SEQ ID NO: 3)) expression levels of DCs co-cultured with MC38-ova cells at a 1:3 ratio, n=6. The legend in FIG. 32I also applies to FIGS. 32F and 32H.

FIGS. 33A-33C: Delivery of pathogen-associated molecular patterns (PAMPs) and dendritic cell (DC) maturation. FIGS. 33A and 33B are graphs showing the interferon-alpha (IFN-α, FIG. 33A) and interleukin-6 (IL-6, FIG. 33B) expression levels quantified by quantitative polymerase chain reaction (qPCR). n=3. The glyceraldehyde-3-phosphate dehydrogenase (GAPDH) was used as a housekeeping gene for comparison of gene expression. DCs were co-cultured with MC38-ova cells pre-treated with free CpG oligodeoxynucleotides (ODNs), Hf-DBB-Ir@CpG, or Hf-DBB^{F}-Ir@CpG plus X-ray irradiation at a dose of 2 Gray (Gy) at a 1:3 ratio. FIG. 33C is a graph showing the quantitative analysis of expression level of Kb-ova of DCs, n=3. Data are expressed as means±s.d. (n=3). *P<0.05 and ***P<0.001 by t-test.

FIGS. 34A-34E: In vivo toxicity and efficacy. Murine colon adenocarcinoma (MC38)-bearing C57BL/6 mice were treated with PBS(−), PBS(+), Hf-DBB^{F}-Ir@CpG(−), CpG(+), Hf-DBB^{F}-Ir(+), Hf-DBB-Ir(+), and Hf-DBB^{F}-Ir@CpG(+), n=6. FIG. 34A is a series of graphs of the immune analyses of tumor microenvironment for 7-day and 14-day MC38 tumor models of 100-150 cubic millimeters (mm³) in sizes, n=6. FIG. 34B is a photo and FIG. 34C and FIG. 34C is a graph of the weights (in grams (g)) of excised tumors of MC38-bearing mice. Pancreatic tumor cell (Panc02)-bearing C57BL/6 mice were treated with PBS(−), PBS(+), Hf-DBB^{F}-Ir@CpG(−), CpG(+), Hf-DBB^{F}-Ir(+), and Hf-DBB^{F}-Ir@CpG(+), n=6. FIG. 34D is a photo and FIG. 34E is a graph of the weights (in g) of excised tumors of Panc02-bearing mice.

FIGS. 35A-35J: Nanoscale metal-organic framework (nMOFs) for in situ personalized cancer vaccination to boost innate immunity for in vivo anti-cancer treatment. FIGS. 35A and 35B are graphs of tumor growth curves of murine colon adenocarcinoma (MC38) (FIG. 35A) and pancreatic cancer (Panc02) (FIG. 35B) tumor-bearing mice treated with PBS(−), PBS(+), CpG(+), Hf-DBB^{F}-Ir(+), Hf-DBB^{F}_Ir@CpG(−), or Hf-DBB^{F}-Ir@CpG(+). Hf-DBB-Ir(+) served as control group on the MC38 model, n=6. FIG. 35C is a graph of the plasma concentration (in nanograms per liter (ng/L) of interleukin-6 (IL-6) and interferon-alpha (IFN-α) 48 hours after first irradiation as quantified by enzyme-linked immunosorbent assay (ELISA). FIGS. 35D and 35E are graphs of the percentages (%) of macrophages (FIG. 35D) and dendritic cells (DCs, FIG. 35E) with respect to the total cells in tumors and tumor-draining lymph nodes (DLN) excised from MC38-bearing mice 2 days post treatment. FIG. 35F is a graph of the percentages of tumor-infiltrating CD80⁺ MHCII⁺ cells with respect to DCs in tumors and DLN excised from MC38-bearing mice day 2 post treatment. FIGS. 35G and 35 H are graphs of the total immunoglobulin G (IgG, FIG. 35G) and total immunoglobulin M (IgM, FIG. 35H) measured in micrograms per liter (g/L) in plasma 2 days and 12 days post irradiation, n=6. FIG. 35I is a graph of the percentages of SIINFEKL (SEQ ID NO: 3)-H₂K^{b+} cells excised from MC38-ova-bearing mice day 6 post treatment. The legend in FIG. 35D also applies to FIGS. 35C and 35E-35I. FIG. 35J is a graph of the tumor growth curves (measured in cubic centimeters (cm³) of MC38-ova tumor-bearing Rag2^{-/-} mice treated with Hf-DBB^{F}_Ir(+) or Hf-DBB^{F}-Ir@CpG(+) with or without OT-I T cell transfer study. Data are expressed as means±s.d. (n=6). n.s. P>0.05, *P<0.05, **P<0.01 and ***P<0.001 by t-test. Central lines, bounds of box and whiskers represent mean values, 25% to 75% of the range of data and 1.5 fold of interquartile range away from outliers, respectively.

FIG. 36: Graph of weight (in milligrams (mg)) of tumor-draining lymph nodes of murine colon adenocarcinoma (MC38)-bearing C57BL/6 mice were treated with PBS(−), PBS(+), Hf-DBB^{F}-Ir@CpG(−), CpG(+), Hf-DBB^{F}-Ir(+), Hf-DBB-Ir(+), Hf-DBB^{F}-Ir@CpG(+). n=6. *P<0.05, **P<0.01, and ***P<0.001 by t-test.

FIGS. 37A-37I: Abscopal effect of in situ cancer vaccination synergized checkpoint blockade immunotherapy (CBI) with promoted adaptive immunity. FIGS. 37A-37C show tumor growth curves of primary treated (FIG. 37A) and distant untreated (FIG. 37B) tumors and survival curves (FIG. 37C) of murine colon adenocarcinoma (MC38)-tumor-bearing mice treated with PBS(−), PBS(+), αPD-L1(+), Hf-DBB^{F}-Ir@CpG(+), Hf-DBB^{F}-Ir@CpG(−)+αPD-L1, or Hf-DBB^{F}-Ir@CpG(+)+αPD-L1. Treatment began on day 14 after tumor inoculation when the tumor reached a volume of 100-150 mm³. X-ray irradiation was carried out on mice 12 h after the i.t. injection of PBS or Hf-DBB^{F}-Ir@CpG on five consecutive days at a dose of 1 gray (Gy)/fraction. Antibody (αPD-L1) was given intraperitoneally every three days at a dose of 75 micrograms (μg)/mouse. n=6. The legend in FIG. 37A also applies to FIG. 37B. FIGS. 37D-37I are graphs showing the percentages of tumor-infiltrating CD45⁺ cells (FIG. 37D), dendritic cells (DCs) (FIG. 37E) CD80⁺ MHCII⁺ DCs (FIG. 37F), natural killer (NK) cells (FIG. 37G), CD4⁺ T cells (FIG. 37H), and CD8⁺ T cells (FIG. 37I) with respect to the total cells 10 days post treatment. Data are expressed as means±s.d. (n=6). *P<0.05, **P<0.01 and ***P<0.001 by t-test. Central lines, bounds of box and whiskers represent mean values, 25% to 75% of the range of data and 1.5 fold of interquartile range away from outliers, respectively. The legend in FIG. 37E also applies to FIGS. 37D and 37F-37I.

FIGS. 38A-38D: Immune analysis on bilateral models. Murine colon adenocarcinoma (MC38)-tumor bearing mice were treated with PBS(−), PBS(+), αPD-L1(+), Hf-DBB^{F}-Ir@CpG(+), Hf-DBB^{F}-Ir@CpG+αPD-L1(−), and Hf-DBB^{F}_Ir@CpG+αPD-L1(+). FIGS. 38A and 38B are graphs showing the percentages (%) of neutrophils (FIG. 38A), or macrophages (FIG. 38B) with respect to the total cells infiltrated into tumors excised from bilateral MC38 tumor-bearing mice 10 days after the first irradiation treatment, n=6. The legend in FIG. 38A also applies to FIG. 38B. FIG. 38C is a photo and FIG. 38D is a graph of the weights (in milligrams (mg)) of excised tumor-draining lymph nodes of mice, n=6.

FIGS. 39A-39J: Specificity and immune memory effect of in situ cancer vaccination plus checkpoint blockade immunotherapy (CBI). FIG. 39A is (left) a series of representative images of colonies (top—control, bottom—stimulated with KSPWFTTL (SEQ ID NO: 5)) and (right) a graph of the statistical analysis of ELISpot assay performed to detect tumor-specific interferon-gamma (IFN-γ) producing T cells (control or stimulated with SEQ ID NO: 5), n=6. FIG. 39B is a schematic illustration of bilateral models established by subcutaneous (s.c.) injection of MC38 and B16F10 or LL2 cells onto flanks as primary and distant tumors, respectively. FIGS. 39C-39F are graphs showing tumor growth curves of primary treated MC38 (FIGS. 39C and 39E) and distant untreated (B16F10 for FIG. 39D and LL2 for FIG. 39F) tumor growth curves on unmatched bilateral tumor models treated with PBS(+), αPD-L1(+), Hf-DBB^{F}-Ir@CpG(+), or Hf-DBB^{F}-Ir@CpG(+)+αPD-L1, n=4. FIGS. 39G and 39H are graphs of primary (FIG. 39G) and distant (FIG. 39H) tumor growth on MC38-bearing Rag2^{-/-} models treated with PBS(−) or Hf-DBB^{F}-Ir@CpG(+)+αPD-L1, n=6. FIG. 39I is a graph of tumor growth curves after challenge with MC38 tumor cells and re-challenge with B16F10 cells on cured mice as treated from FIG. 37C. FIG. 39J is a graph of percentages (%) of CD44^{high}CD62L^{low} cells with respect to the total splenocytes, n=6. The legend in the graph of FIG. 39A also applies to FIG. 39J.

FIG. 40: Graph showing quantified fluoroscein-isothiocyanate (FITC) signal from FITC-mucin 1 (MUC-1) per cell by confocal laser scanning microscopy (CLSM), N=3. CLSM showed that FITC-MUC-1/Hf-DBP-Pt delivered MUC-1 peptides into HEK293T cells efficiently.

FIG. 41: Graph of 3-(4,5-dimethylthiazol-2-yl)-5-(3-carboxymethoxyphenyl)-2-(4-sulfo-phenyl)-2H-tetrazolium (MTS) assay results showing that mucin-1 (MUC-1) peptide can synergize with radiotherapy-radiodynamic therapy (RT-RDT) by hafnium-5,15-di(p-benzoato)porphyrin (Hf-DBP) with platinum (Pt) (Hf-DBP-Pt) to better kill cancer cells (murine colon adenocarcinoma (MC38) cells) in vitro upon X ray irradiation.

FIG. 42: Graph of tumor growth curves (tumors measured in cubic millimeters (mm³)) of murine colon adenocarcinoma (MC38) bearing C57BL/6 mice treated by phosphate buffered saline (PBS), mucin-1 peptide (MUC-1) and a metal-organic framework with surface associated MUC-1 (i.e., Hf-DBP-Pt/MUC-1) intratumorally. All mice received 1 gray (Gy) X-ray each day in 6 consecutive days with minimal toxicity of MUC-1/Hf-DBP-Pt system.

FIG. 43: Graph of CpG oligodeoxynucleotide loading percentages by different nMOFs.

FIG. 44. Graph of tumor growth curves (tumors measured in cubic millimeters (mm³)) of murine colon adenocarcinoma (MC38)-tumor-bearing C57BL/6 mice treated with phosphate buffered saline (PBS), cyclic guanosine monophosphate-adenosine monophosphate (cGAMP) or a metal-organic framework with surface associated cGAMP (cGAMP/Hf-DBP-Pt) intratumorally. All mice received 2 gray (Gy) X-day each day in 5 consecutive days starting on Day 7. The cGAMP/Hf-DBP-Pt system has minimal systemic toxicity as indicated by the steady body weight trend.

FIGS. 45A and 45B: Pair of graphs of cyclic guanosine monophosphate-adenosine monophosphate (cGAMP) adsorption percentage (FIG. 45A) and cGAMP release profiles (FIG. 45B) of cGAMP/nanoscale metal-organic layer (nMOL) in different buffers.

FIG. 46: Graph of isothermal titration calorimetry (ITC) fitting result of cyclic guanosine monophosphate-adenosine monophosphate (cGAMP) titration into the nanoscale metal-organic layer (nMOL) in water solution.

FIG. 47: Graph of interferon regulatory factor (IRF) response measured by THP1-DUAL™ KO-MyD88 reporter cells (InvivoGen, San Diego, California, United States of America). Cyclic guanosine monophosphate-adenosine monophosphate (cGAMP)/nanoscale metal-organic layer (nMOL) had a much lower half maximal effective concentration (EC₅₀) with higher IRF response level than free 2′,3′-cGAMP.

FIG. 48. Quantification of fluorescence signals after intratumoral injection of either cyclic guanosine monophosphate-adenosine monophosphate (cGAMP)-Cy5 or cGAMP-Cy5/nanoscale metal-organic layer (nMOL). The nMOL retained and protected cGAMP for a much longer period of time than free cGAMP.

FIGS. 49A and 49B: Graphs of tumor growth curves (tumors measured in cubic centimeters (cm³)) of murine colon adenocarcinoma (MC38)-tumor bearing C57BL/6 mice (FIG. 49A) and murine colorectal carcinoma (CT26)-tumor bearing BALB/c mice (FIG. 49B) treated by phosphate buffered saline (PBS), cyclic guanosine monophosphate-adenosine monophosphate (cGAMP), nanoscale metal-organic layer (nMOL) and cGAMP/nMOL with (+) or without (−) irradiation. All mice received 2 gray (Gy) X-ray each day starting on Day 7 for 6 consecutive days. The cGAMP/nMOL system showed minimal toxicity.

FIGS. 50A and 50B: Graphs showing tumor growth curves (tumors measured in cubic centimeters (cm³)) of bilateral murine colon adenocarcinoma (MC38)-bearing C57BL/6 mice treated by phosphate buffered saline (PBS), anti-programming death ligand 1 antibody (αPD-L1), a nanoscale metal-organic layer with surface associated cyclic guanosine monophosphate-adenosine monophosphate (cGAMP/nMOL), and αPD-L1+cGAMP/nMOL with irradiation. FIG. 50A is a graph of the tumor growth curves for the primary tumor. FIG. 50B is a graph of the tumor growth curves for the distant tumor. All mice received 2 Gy X ray each day starting on Day 7 for 6 consecutive days. αPD-L1 was intraperitoneally injected on Day 10 and 13.

### REFERENCE TO SEQUENCE LISTING SUBMITTED ELECTRONICALLY

The content of the electronically submitted sequence listing in ASCII text file (Name: 3072-19-PCT.ST25.txt; Size: 2 kilobytes; and Date of Creation: May 21, 2021) filed with the application is incorporated herein by reference in its entirety.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. Although any methods, devices, and materials similar or equivalent to those described herein can be used in the practice or testing of the presently disclosed subject matter, representative methods, devices, and materials are now described. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a metal ion” includes a plurality of such metal ions, and so forth.

Unless otherwise indicated, all numbers expressing quantities of size, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of size (i.e., diameter), weight, concentration or percentage is meant to encompass variations of in one example ±20% or ±10%, in another example ±5%, in another example ±1%, and in still another example ±0.1% from the specified amount, as such variations are appropriate to perform the disclosed methods.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein the term “alkyl” can refer to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. In some embodiments, there can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Heteroaryl” as used herein refers to an aryl group that contains one or more non-carbon atoms (e.g., O, N, S, Se, etc) in the backbone of a ring structure. Nitrogen-containing heteroaryl moieties include, but are not limited to, pyridine, imidazole, benzimidazole, pyrazole, pyrazine, triazine, pyrimidine, and the like.

“Aralkyl” refers to an -alkyl-aryl group, optionally wherein the alkyl and/or aryl moiety is substituted.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “arylene” refers to a bivalent aromatic group, e.g., a bivalent phenyl or napthyl group. The arylene group can optionally be substituted with one or more aryl group substituents and/or include one or more heteroatoms.

The term “amino” refers to the group —N(R)₂ wherein each R is independently H, alkyl, substituted alkyl, aryl, substituted aryl, aralkyl, or substituted aralkyl. The terms “aminoalkyl” and “alkylamino” can refer to the group —N(R)₂ wherein each R is H, alkyl or substituted alkyl, and wherein at least one R is alkyl or substituted alkyl. “Arylamine” and “aminoaryl” refer to the group —N(R)₂ wherein each R is H, aryl, or substituted aryl, and wherein at least one R is aryl or substituted aryl, e.g., aniline (i.e., —NHC₆H₅).

The term “thioalkyl” can refer to the group —SR, wherein R is selected from H, alkyl, substituted alkyl, aralkyl, substituted aralkyl, aryl, and substituted aryl. Similarly, the terms “thioaralkyl” and “thioaryl” refer to —SR groups wherein R is aralkyl and aryl, respectively.

The terms “halo”, “halide”, or “halogen” as used herein refer to fluoro, chloro, bromo, and iodo groups.

The “haloalkyl” refers to an alkyl group substituted by one or more halo groups. A “perhaloalkyl” group refers to an alkyl group where all of the hydrogen atoms that are attached to carbon atoms are replaced by halo groups. An exemplary perhaloalkyl group is trifluoromethyl (i.e., —CF₃).

The terms “hydroxyl” and “hydroxy” refer to the —OH group.

The terms “mercapto” or “thiol” refer to the —SH group.

The terms “carboxylate” and “carboxylic acid” can refer to the groups —C(═O)O⁻ and —C(═O)OH, respectively. The term “carboxyl” can also refer to the —C(═O)OH group. In some embodiments, “carboxylate” or “carboxyl” can refer to either the —C(═O)O⁻ or —C(═O)OH group. In some embodiments, when the term “carboxylate” is used in reference to an anion of a SBU, the term “carboxylate” can be used to refer to the anion HCO₂⁻ and, thus, can be synonymous with the term “formate”.

The term “carbonyl” refers to the —C(═O)—R group where R is H, alkyl, substituted alkyl, aralkyl, substituted aralkyl, aryl, or substituted aryl.

The term “phosphonate” refers to the —P(═O)(OR)₂ group, wherein each R can be independently H, alkyl, aralkyl, aryl, or a negative charge (i.e., wherein effectively there is no R group present to bond to the oxygen atom, resulting in the presence of an unshared pair of electrons on the oxygen atom). Thus, stated another way, each R can be present or absent, and when present is selected from H, alkyl, aralkyl, or aryl.

The term “phosphate” refers to the —OP(═O)(OR′)₂ group, where R′ is H or a negative charge.

The term “lipid” can refer to a hydrophobic or amphiphilic small molecule, such as, but not limited to a fatty acid, a phospholipid, a glycerolipid, a glycerophospholipid, a sphingolipid, a saccharolipid, or a polyketide.

The terms “bonding” or “bonded” and variations thereof can refer to either covalent or non-covalent bonding (e.g., hydrogen bonding, ionic bonding, van der Waals interactions, etc.). In some cases, the term “bonding” refers to bonding via a coordinate bond or via electrostatic interactions.

The term “conjugation” can refer to a bonding process, as well, such as the formation of a covalent linkage or a coordinate bond.

As used herein, the term “metal-organic framework” or “MOF” refers to a solid two- or three-dimensional network comprising both metal and organic components, wherein the organic components include at least one, and typically more than one carbon atom. In some embodiments, the material is crystalline. In some embodiments, the material is amorphous. In some embodiments, the material is porous. In some embodiments, the metal-organic matrix material is a coordination polymer, which comprises repeating units of coordination complexes comprising a metal-based secondary building unit (SBU), such as a metal ion or metal complex, and a bridging polydentate (e.g., bidentate or tridentate) organic ligand. In some embodiments, the material contains more than one type of SBU or metal ion. In some embodiments, the material can contain more than one type of organic bridging ligand.

The term “nanoscale metal-organic framework” can refer to a nanoscale structure comprising, consisting essentially of, or consisting of an MOF.

The terms “nanoscale,” “nanomaterial,” and “nanoparticle” refer to a structure having at least one region with a dimension (e.g., length, width, diameter, etc.) of less than about 1,000 nm. In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, or even less than about 30 nm). In some embodiments, the dimension is between about 30 nm and about 250 nm (e.g., about 30, 40, 50, 60, 70, 80, 90, 100, 110, 120, 130, 140, 150, 160, 170, 180, 190, 200, 210, 220, 230, 240, or 250 nm).

In some embodiments, a nanoparticle is approximately spherical. When the nanoparticle is approximately spherical, the characteristic dimension can correspond to the diameter of the sphere. In addition to spherical shapes, the nanomaterials/nanoparticles can be disc-shaped, plate-shaped (e.g., hexagonally plate-like), oblong, polyhedral, rod-shaped, cubic, or irregularly-shaped.

As used herein, the terms “nanoplate”, “metal-organic nanoplates”, and “MOP” refer to a MOF with a plate- or disc-like shape, i.e., wherein the MOF is substantially longer and wider than it is thick. In some embodiments, the MOP is at least 2, 3, 4, 5, 6, 7, 8, 9, 10, 25, or 50 times longer and/or wider than it is thick. In some embodiments, the MOP is less than about 100 nm, 50 nm, or about 30 nm thick. In some embodiments, the MOP is between about 3 nm and about 30 nm thick (e.g., about 5, 10, 15, 20, 25, or 30 nm thick). In some embodiments, the MOP is between about 3 nm and about 12 nm thick (e.g., 3, 4, 5, 6, 7, 8, 9, 10, 11, or 12 nm thick). In some embodiments, the MOP is about two, three, four, five, six, seven, eight, nine or ten layers thick. In some embodiments, the MOP is between about two and about five layers thick, wherein each layer is about the thickness of a SBU. In some embodiments, the MOP is crystalline. In some embodiments, the MOP is amorphous. In some embodiments, the MOP is porous. In some embodiments, a strongly coordinating modulator, such as a monocarboxylic acid like acetic acid (AcOH), formic acid, benzoic acid, or trifluoroacetic acid (TFA), is used to control the nanoplate morphology of the MOP and to introduce defects (missing bridging ligands) to enhance the diffusion of ROS through MOP channels.

As used herein, the term “metal-organic layer” (or “MOL”) refers to a solid, mainly two-dimensional network comprising both metal and organic components, wherein the organic components include at least one, and typically more than one carbon atom. In some embodiments, the MOL is crystalline. In some embodiments, the MOL is amorphous. In some embodiments, the MOL is porous.

In some embodiments, the MOL is a coordination polymer, which comprises repeating units of coordination complexes comprising a metal-based secondary building unit (SBU), such as a metal ion or metal complex, and a bridging polydentate (e.g., bidentate or tridentate) organic ligand. In some embodiments, the bridging ligand is essentially planar. In some embodiments, a majority of bridging ligands bind to at least three SBUs. In some embodiments, the material contains more than one type of SBU or metal ion. In some embodiments, the material can contain more than one type of bridging ligand.

In some embodiments, the MOL can be essentially a monolayer of a coordination complex network between the SBUs and the bridging ligands where the monolayer extends in the x- and y-planes but has a thickness of only about one SBU.

In some embodiments, the MOL can be a monolayer of a substantially planar coordination complex network between the SBUs and the bridging ligands wherein substantially all of the bridging ligands are in the same plane. In some embodiments, more than 80%, 85%, 90%, or 95% of the bridging ligands are substantially in the same plane. In some embodiments, more than 95%, 96%, 97%, 98%, 99%, or about 100% of the bridging ligands are in the same plane. Thus, while the MOL can extend in the x- and y-planes for a distance that can comprise the length and/or diameter of multiple SBUs and bridging ligands, in some embodiments, the MOL can have a thickness of only about one SBU. In some embodiments, the thickness of the MOL is about 3 nm or less (e.g., about 3, 2, or about 1 nm or less) and the width, length, and/or diameter of the MOL is at least about 5 times, 10 times, 20 times, 30 times, 40 times, 50 times, 60 times, 70 times, 80 times, 90 times, or about 100 times or more the thickness of the MOL. In some embodiments, the MOL has a sheet-like shape. In some embodiments, the MOL has a plate-like or disc-like shape. In some embodiments, a coordinating modulator, such as a monocarboxylic acid like acetic acid (AcOH), formic acid, benzoic acid, or trifluoroacetic acid (TFA), is used to control the nanoplate morphology of the MOL and/or to introduce defects (missing bridging ligands) to enhance the diffusion of ROS through MOL channels or modulate other properties of the MOL.

In contrast to an MOL and/or MOP, the terms “metal-organic framework”, “nanoscale metal-organic framework”, “MOF” and/or “nMOF” as used herein typically refer to a solid metal-organic matrix material particle wherein each of the length, width, thickness, and/or diameter of the MOF is greater than about 30 or 31 nm (or greater than about 50 nm or greater than about 100 nm) and/or wherein none of the width, length, and/or diameter of the MOF is 5 or more times greater than the thickness of the MOF.

A “coordination complex” is a compound in which there is a coordinate bond between a metal ion and an electron pair donor, ligand or chelating group. Thus, ligands or chelating groups are generally electron pair donors, molecules or molecular ions having unshared electron pairs available for donation to a metal ion.

The term “coordinate bond” refers to an interaction between an electron pair donor and a coordination site on a metal ion resulting in an attractive force between the electron pair donor and the metal ion. The use of this term is not intended to be limiting, in so much as certain coordinate bonds also can be classified as having more or less covalent character (if not entirely covalent character) depending on the characteristics of the metal ion and the electron pair donor.

As used herein, the term “ligand” refers generally to a species, such as a molecule or ion, which interacts, e.g., binds, in some way with another species. More particularly, as used herein, a “ligand” can refer to a molecule or ion that binds a metal ion in solution to form a “coordination complex.” See Martell, A. E., and Hancock, R. D, Metal Complexes in Aqueous Solutions, Plenum: New York (1996), which is incorporated herein by reference in its entirety. The terms “ligand” and “chelating group” can be used interchangeably. The term “bridging ligand” can refer to a group that bonds to more than one metal ion or complex, thus providing a “bridge” between the metal ions or complexes. Organic bridging ligands can have two or more groups with unshared electron pairs separated by, for example, an alkylene or arylene group. Groups with unshared electron pairs, include, but are not limited to, —CO₂H, —NO₂, amino, hydroxyl, thio, thioalkyl, —B(OH)₂, —SO₃H, PO₃H, phosphonate, and heteroatoms (e.g., nitrogen, oxygen, or sulfur) in heterocycles.

The term “coordination site” when used herein with regard to a ligand, e.g., a bridging ligand, refers to a unshared electron pair, a negative charge, or atoms or functional groups cable of forming an unshared electron pair or negative charge (e.g., via deprotonation under at a particular pH).

“Electrostatic bonding” refers to attractive forces between two completely or partially ionized species with opposite charges.

The term “small molecule” refers to a non-polymeric, naturally-occurring or synthetic molecule. Small molecules typically have a molecular weight of about 900 Daltons (Da) or less (e.g., about 800 Da, about 750 Da, about 700 Da, about 650 Da, about 600 Da, about 550 Da, or about 500 Da or less).

The term “macromolecule” as used herein refers to molecules that are larger than about 900 Da. In some embodiments, the macromolecule is a polymer or biopolymer, e.g., a protein or a nucleic acid.

The terms “polymer” and “polymeric” refer to chemical structures that have repeating units (i.e., multiple copies of a given chemical substructure). Polymers can be formed from polymerizable monomers. A polymerizable monomer is a molecule that comprises one or more moieties that can react to form bonds (e.g., covalent or coordination bonds) with moieties on other molecules of polymerizable monomer. Generally, each polymerizable monomer molecule can bond to two or more other molecules. In some cases, a polymerizable monomer will bond to only one other molecule, forming a terminus of the polymeric material.

Polymers can be organic, or inorganic, or a combination thereof. As used herein, the term “inorganic” refers to a compound or composition that contains at least some atoms other than carbon, hydrogen, nitrogen, oxygen, sulfur, phosphorous, or one of the halides. Thus, for example, an inorganic compound or composition can contain one or more silicon atoms and/or one or more metal atoms.

As used herein “organic polymers” are those that do not include silica or metal atoms in their repeating units. Exemplary organic polymers include polyvinylpyrrolidone (PVO), polyesters, polyamides, polyethers, polydienes, and the like. Some organic polymers contain biodegradable linkages, such as esters or amides, such that they can degrade overtime under biological conditions.

The term “hydrophilic polymer” as used herein generally refers to hydrophilic organic polymers, such as but not limited to, polyvinylpyrrolidone (PVP), polyvinylmethylether, polymethyloxazoline, polyethyloxazoline, polyhydroxy-propyloxazoline, polyhydroxypropylmethacrylamide, polymethyacrylamide, polydimethylacrylamide, polyhydroxylpropylmethacrylate, polyhydroxy-ethylacrylate, hydroxymethylcellulose, hydroxyethylcellulose, polyethylene-imine (PEI), polyethyleneglycol (i.e., PEG) or another hydrophilic poly(alkyleneoxide), polyglycerine, and polyaspartamide. The term “hydrophilic” refers to the ability of a molecule or chemical species to interact with water. Thus, hydrophilic polymers are typically polar or have groups that can hydrogen bond to water.

“Polypeptide” and “peptide” refer to a polymer composed of amino acid residues linked via peptide (amide) bonds, related naturally occurring structural variants, and synthetic non-naturally occurring analogs thereof. In some embodiments, “peptide” refers to a polymer composed of between 2 and 50 amino acid residues.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “protein” typically refers to large polypeptides (e.g., >50 amino acid residues). Conventional notation is used herein to portray polypeptide sequences: the left-hand end of a polypeptide sequence is the amino-terminus; the right-hand end of a polypeptide sequence is the carboxyl-terminus.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

In some embodiments, a “preventive” or “prophylactic” treatment is a treatment administered to a subject who does not exhibit signs, or exhibits only early signs, of a condition, disease, or disorder. Thus, a prophylactic or preventative treatment can be administered for the purpose of decreasing the risk of developing pathology associated with developing the condition, disease, or disorder.

The term “photosensitizer” (PS) refers to a chemical compound or moiety that can be excited by light of a particular wavelength, typically visible or near-infrared (NIR) light, and produce a reactive oxygen species (ROS). For example, in its excited state, the photosensitizer can undergo intersystem crossing and transfer energy to oxygen (O₂) (e.g., in tissues being treated by PDT) to produce ROSs, such as singlet oxygen (¹O₂).

In some embodiments, the photosensitizer is a porphyrin, a chlorophyll, a dye, or a derivative or analog thereof, such as a porphyrin, chlorophyll or dye comprising one or more additional aryl or alkyl group substituents, having one or more carbon-carbon double bonds replaced by a carbon-carbon single bond, and/or comprising a substituent (e.g., a substituted alkylene group) that can covalently substituted with a bond to an organic bridging ligand). In some embodiments, porphyrins, chlorins, bacteriochlorins, or porphycenes can be used. In some embodiments, the photosensitizer can have one or more functional groups, such as carboxylic acid, amine, or isothiocyanate, e.g., for using in attaching the photosensitizer to another molecule or moiety, such as an organic bridging ligand or a SBU, and/or for providing an additional site or sites to enhance coordination or to coordinate an additional metal or metals. In some embodiments, the photosensitizer is a porphyrin or a derivative or analog thereof. Exemplary porphyrins include, but are not limited to, hematoporphyrin, protoporphyrin and tetraphenylporphyrin (TPP). Exemplary porphyrin derivatives include, but are not limited to, pyropheophorbides, bacteriochlorophylls, chlorophyll a, benzoporphyrin derivatives, tetrahydroxyphenyl chlorins, purpurins, benzochlorins, naphthochlorins, verdins, rhodins, oxochlorins, azachlorins, bacteriochlorins, tolyporphyrins and benzobacteriochlorins. Porphyrin analogs include, but are not limited to, expanded porphyrin family members (such as texaphyrins, sapphyrins and hexaphyrins), porphyrin isomers (such as porphycenes, inverted porphyrins, phthalocyanines, and naphthalocyanines), and TPP substituted with one or more functional groups.

In some embodiments, the PS is a metal coordination complex comprising a metal (e.g., Ru or Ir) and one or more nitrogen donor ligands, e.g., one or more nitrogen-containing aromatic groups. In some embodiments, the one or more nitrogen donor ligands are selected from the group including, but not limited to, a bipyridine (bpy), a phenanthroline, a terpyridine, or a phenyl-pyridine (ppy), each of which can optionally be substituted with one or more aryl group substituents (e.g., on a carbon atom of the aromatic group).

The term “cancer” as used herein refers to diseases caused by uncontrolled cell division and/or the ability of cells to metastasize, or to establish new growth in additional sites. The terms “malignant”, “malignancy”, “neoplasm”, “tumor,” “cancer” and variations thereof refer to cancerous cells or groups of cancerous cells.

Particular types of cancer include, but are not limited to, skin cancers (e.g., melanoma), connective tissue cancers (e.g., sarcomas), adipose cancers, breast cancers, head and neck cancers, lung cancers (e.g., mesothelioma), stomach cancers, pancreatic cancers, ovarian cancers, cervical cancers, uterine cancers, anogenital cancers (e.g., testicular cancer), kidney cancers, bladder cancers, colorectal cancers (i.e., colon cancers or rectal cancers), prostate cancers, central nervous system (CNS) cancers, retinal cancer, blood, neuroblastomas, multiple myeloma, and lymphoid cancers (e.g., Hodgkin's and non-Hodgkin's lymphomas).

The term “metastatic cancer” refers to cancer that has spread from its initial site (i.e., the primary site) in a patient's body.

The terms “anticancer drug”, “chemotherapeutic”, and “anticancer prodrug” refer to drugs (i.e., chemical compounds) or prodrugs known to, or suspected of being able to treat a cancer (i.e., to kill cancer cells, prohibit proliferation of cancer cells, or treat a symptom related to cancer). In some embodiments, the term “chemotherapeutic” as used herein refers to a non-PS molecule that is used to treat cancer and/or that has cytotoxic ability. Such more traditional or conventional chemotherapeutic agents can be described by mechanism of action or by chemical compound class, and can include, but are not limited to, alkylating agents (e.g., melphalan), anthracyclines (e.g., doxorubicin), cytoskeletal disruptors (e.g., paclitaxel), epothilones, histone deacetylase inhibitors (e.g., vorinostat), inhibitors of topoisomerase I or II (e.g., irinotecan or etoposide), kinase inhibitors (e.g., bortezomib), nucleotide analogs or precursors thereof (e.g., methotrexate), peptide antibiotics (e.g., bleomycin), platinum based agents (e.g., cisplatin or oxaliplatin), retinoids (e.g., tretinoin), and vinka alkaloids (e.g., vinblastine).

### II. General Considerations

For several decades, cancer vaccines have been developed to amplify tumor-specific T cell responses (Goldman and DeFrancesco, 2009). In particular, tumor antigen-based cancer vaccines have been widely investigated in the clinic, leading to the approval of the prostatic acid phosphatase-based prostate cancer vaccine Sipuleucel-T by the United States Food and Drug Administration (Hu et al., 2018). However, traditional approaches to cancer vaccine development face several hurdles, including tumor heterogeneity with different somatic mutations and hence varied tumor antigens among patients (Tanyi et al., 2018; Sahin and Türeci, 2018), ineffective delivery of peptide-based tumor antigens to lymph nodes due to rapid renal clearance and enzymatic degradation (Purcell et al., 2007; Du et al., 2018), inefficient internalization of tumor antigens by antigen presenting cells (APCs) (Liu et al., 2014), and the ability of tumors to escape from immune surveillance via mechanisms such as the programming death-1/programming death-ligand 1(PD-1/PD-L1) axis (Joyce and Fearon, 2015).

Personalized vaccines with neo-antigens or autologous whole tumor lysates can overcome tumor heterogeneity (Kuai et al., 2017), but their production processes are lengthy, complicated, and expensive (Scheetz et al., 2019). One promising strategy to improve personalized cancer vaccination uses immunostimulatory treatments to generate tumor antigens in situ, which can afford systemic antitumor immune responses in a personalized fashion and modulate local tumor microenvironments to relieve immunosuppression (Wang et al., 2018). For instance, intratumoral injection of oncolytic viruses such as talimogene laherparepvec (T-VEC) inflicts direct cytotoxic effects on cancer cells and recruits DCs for antigen presentation, acting as in situ cancer vaccines with reduced side effects (Gilliet et al., 2008). Non-viral treatments with potent antitumor effects, such as phototherapy (Castano et al., 2006), radiotherapy (RT) (Weichselbaum et al., 2014; Chao et al., 2018), and some chemotherapies can also generate danger-associated molecular patterns (DAMPs) and tumor antigens by inducing immunogenic cell death (Kepp et al., 2019).

Stimulation of DCs with immunoadjuvants such as stimulator of interferon genes (STING) agonist (Shae et al., 2019; Luo et al., 2017) or CpG oligodeoxynucleotides (ODNs) further promotes antigen presentation and immune responses (Klinman, 2004). Naturally existing as microbial DNAs known as pathogen-associated molecular patterns (PAMPs), CpGs are short DNA strands explored widely as vaccine adjuvants for toll-like receptor 9 (TLR9) stimulation, DC maturation, antigen presentation, and the priming of tumor-specific cytotoxic T lymphocytes (CTL) (Figdor et al., 2004). In addition, antigen presentation by immature DCs in the absence of immunoadjuvants induces tolerance rather than stimulates an immune reaction (Lutz and Schuler, 2002). In particular, class C CpGs can enhance Type I interferon (IFN) production to activate DCs and stimulate B cells, which in turn upregulates co-stimulatory molecules and secretes pro-inflammatory cytokines to afford superb anticancer effects (Radovic-Moreno et al., 2015; Brody et al., 2010). Similar to peptide vaccines, however, even locally administrated CpGs are prone to enzymatic degradation and cannot be efficiently internalized by APCs due to their anionic nature (Brody et al., 2010; Rosi et al., 2006).

In the context of antigen and adjuvant delivery, nanotechnology is at the forefront of merging drug delivery and immune stimulation to elicit antitumor activity for cancer vaccination (Jiang et al., 2017; Nam et al., 2019; Song et al., 2017; Zhang et al., 2019; Ma et al., 2019; Lou et al., 2019; and Wilson et al., 2019). The presently disclosed subject matter is based, in one aspect, on the use of nanoscale metal-organic frameworks (nMOFs) for personalized cancer vaccination via X-ray activated generation of DAMPs and tumor antigens and efficient delivery of CpGs to APCs as PAMPs. However, CpGs are merely one exemplary agent that can be delivered by the nMOFs. Other immunotherapy agents, including an array of small molecules or macromolecules can be delivered using nMOFs according to the presently disclosed subject matter, as described further hereinbelow.

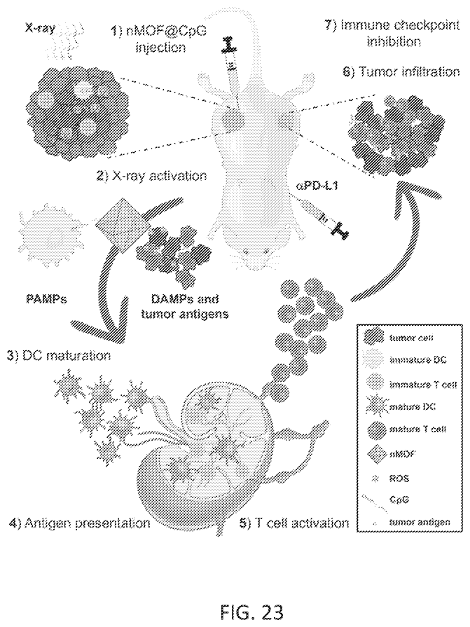

Assembled from tunable metal-oxo clusters and functional organic ligands, nMOFs have emerged as a new type of porous and crystalline molecular nanomaterials with interesting potential in biomedical applications (Furukawa et al., 2013; Wang et al, 2017). nMOFs have shown potent antitumor activity by generating highly cytotoxic and immunogenic reactive oxygen species (ROSs) upon external light or X-ray irradiation (Lan et al., 2019a; Ni et al., 2018a). nMOFs are also able to directly convert X-ray energy to ROSs via a unique radiotherapy-radiodynamic therapy (RT-RDT) (Ni et al., 2018b). According to one aspect of the presently disclosed subject matter cationic nMOFs have been designed through molecular engineering to release DAMPs and tumor antigens via X-ray activated RT-RDT and, thus, for example, to deliver CpGs via electrostatic interactions. The in situ vaccination afforded by nMOFs effectively expand cytotoxic T cells in tumor-draining lymph nodes to reinvigorate the adaptive immune system for tumor regression. See FIG. 23. The local therapeutic effects of the nMOF-based in situ vaccines were extended to distant tumors by combination treatment with an anti-PD-L1 antibody (αPD-L1) to afford an 83.3% cure rate on an MC38 colorectal cancer model.

Also described herein are additional exemplary nMOF for treating cancer by activating the immune system. For example, described hereinbelow is a nMOF that co-delivers (i) the Toll-like receptor 7/8 (TLR7/8) agonist imiquimod (IMD) as a pathogen-associated molecular pattern (PAMP), and (ii) an anti-cluster of differentiation 46 antibody (αCD47) for macrophage modulation and reversal of immunosuppression. See FIG. 1. IMD repolarizes immunosuppressive type M2 macrophages to immunostimulatory M1 macrophages, while the antibody blocks CD47 tumor cell surface marker to promote phagocytosis. The IMD can be sequestered in pores in the nMOF core while the surface of the nMOF is modified to enhance its ability to bond to the antibody. nMOFs combining delivery of IMD and αCD47 were injected intratumorally followed by low grade X-ray irradiation and showed enhanced tumor regression over αCD47, IMD, or bare nMOF treatments alone. Upon X-ray irradiation, the nMOF-triggered RT-RDT, IMD and αCD47 together modulate immunosuppressive tumor microenvironment and active innate immunity to orchestrate adaptive immunity when used in combination with an anti-PDL1 immune checkpoint inhibitor, which can lead to complete eradication of both primary and distant tumors in a bilateral colorectal tumor model. Thus, nMOFs provide an effective platform to co-deliver multiple immunoadjuvants to induce systemic immune response and provide improved anti-tumor efficacy.

### III. Nanoscale Metal-Organic Frameworks

In some embodiments, the presently disclosed subject matter relates to metal-organic frameworks (MOFs) (e.g., nanoscale MOFs (nMOFs), such as MOF nanoparticles or nanoscale metal-organic layers (MOLs)) that are modified to be capable of improved surface absorption of therapeutic agents compared to a parent, non-modified MOF. The modified MOFs can be capable of increased surface absorption of small molecules and/or macromolecule therapeutic agents. The surface absorption can involve coordinative bonding of the therapeutic agent to a metal ion at the surface of the MOF or electrostatic interactions between the therapeutic agent and the surface of the MOF. In some embodiments, these therapeutic agents can include, but are not limited to: (1) nucleic acids such as microRNA (miRNA), messenger (mRNA), small interfering RNA (siRNA), and CpG ODN; (2) polynucleotides and cyclic di-nucleotides (such as STING agonists c-di-AMP and cGAMP); (3) small molecules and macromolecules with accessible phosphate or carboxyl groups (such as etoposide phosphate); (4) peptides and proteins such as anti-CD37, anti-CD44, anti-CD47, anti-CD73, anti-PD-1, anti-PD-L1, anti-LAG3, anti-CTLA-4 antibodies; and (5) combinations of any of (1)-(4).

In some embodiments, the therapeutic agents target the immune system. Such therapeutic agents include, but are not limited to, PAMPS, such as Toll-like receptor (TLR) agonists and RIG-I-like receptor (RLR) agonists and STING agonists; oncolytic molecules, including both small molecule oncolytic molecules such as PV-10, targeting lysosomes, and peptides, such as ruxotemitide, targeting mitochondria; cytokines, including but not limited to interleukins (ILs) (e.g., IL-2, IL-7, IL-12, IL-15 and other interleukins and their natural or synthetic derivatives), interferons (IFNs) (e.g., Type I, II, III IFNs including IFN-α, IFN-β, IFN-γ and other IFNs, and their natural or synthetic derivatives), and tumor necrosis factors (TNF) (e.g., TNF-α and their natural or synthetic derivatives); antibodies including monoclonal antibodies (mAbs) that target PD-1, PD-L1, CTLA, CD47, SIRPa, CD28, OX40, CD127, and CD40, as well as bispecific monoclonal antibodies; and nucleic acids, such as, but not limited to siRNA, miRNA, mRNA, and non-coding RNA (ncRNA). Exemplary TLR agonists and RLR agonists include TLR3 agonists, such as poly(I:C), poly(A:U), double-stranded RNA (dsRNA), and their natural or synthetic derivatives; TLR4 agonists, such as lipopolysaccharides (LPS), and their natural and synthetic derivatives like monophosphoryl Lipid A (MPLA), CRX-527, G100 etc.; TLR7/8 agonists, including imidazoquinoline compounds such as imiquimod (IMD), gardiquimod, resiquimod, CL075, CL097, CL264, CL307; benzoazepine analogs such as VTX-2337, TL8-506; single stranded RNA (ssRNA, and their natural and synthetic derivatives; TLR-9 agonists, such as CpG oligonucleotides (ODNs) including class A, B, and C CpG ODNs, double stranded DNA (dsDNA), and their natural and synthetic derivatives; and RLR ligands to target RIG-I like receptors (RLRs), e.g., dsRNA such as 5′ppp-dsRNA; 3p-hairpin RNA; Poly(dA:dT); Poly(I:C); and their natural and synthetic derivatives. Exemplary STING agonists include, but are not limited to, cyclic dinucleotides (CDNs), including natural CDNs such as 2′3′-cGAMP, 3′3′-cGAMP, c-di-AMP, c-di-GMP; cyclic adenine monophosphate-inosine monophosphate (cAIMP); xanthenone analog such as DMXAA; and their natural and synthetic derivatives such as CL656 and ADU-S100; and phosphodiesterase inhibitors, such as ENPP1/2 inhibitors and SVPD inhibitors.

### III.A. Modified Metal-Organic Frameworks

In some embodiments, the presently disclosed subject matter relates to a modified metal-organic framework (MOF), e.g., a nMOF, comprising metal-containing secondary building units (SBUs) linked together via organic bridging ligands. For example, an organic bridging ligand can include at least two functional groups (e.g., a carboxylate or a phosphate) capable of forming a coordinative bond to a metal ion and can link two SBUs together by forming coordinative bonds with a metal ion on each of the two SBUs with one of these two functional groups. In some embodiments, the SBUs are metal oxo clusters. In some embodiments, the metal ion of the SBU is capable of absorbing x-rays. In the parent, unmodified MOF, the SBU can include a strongly coordinating capping group coordinated to a metal ion, such as an acetate or a formate group. This strongly coordinating capping group can be removed via treatment with trimethysilyl trifluoroacetate, trimethysilyl triflate, or with mineral acids with suitable acid strength, thereby exchanging the strongly coordinating capping groups with weakly coordinating groups, such as trifluoroacetate or triflate groups, in the modified MOF. The weakly coordinating groups can then be replaced by the carboxylate group of a protein or carboxylate group-containing small molecule or a phosphate group of a nucleic acid, attaching the protein, peptide, small molecule, or nucleic acid to the surface of the MOF via coordination to the metal ion. Alternatively, a combination of electron-withdrawing bridging ligands can be used to increase defects of the capping groups and cationic charges on the bridging ligands to increase electrostatic interactions between the MOF and macromolecules. Thus, the term “modified MOF” also refers to MOFs with electron-withdrawing bridging ligands. Methods of preparing parent MOFs to be modified are described, for example, in U.S. Pat. No. 10,206,871, the disclosure of which is incorporated herein by reference in its entirety.

The modified MOFs can act as delivery platforms for a one or more MOF surface-attached therapeutic agents (e.g., one or more therapeutic agents that targets the immune system). In some embodiments, the modified MOF can provide improved absorption of the surface-attached therapeutic agents. In addition, the pores/cavities of the MOFs can be further loaded with small molecule chemotherapeutic agents, such as, but not limited to, cisplatin, carboplatin, paclitaxel, SN-35, etoposide, and the like; or small molecule inhibitors, such as, but not limited to, a PLK1 inhibitor (TAK-960, ON01910, BI 2536, etc.), a Wnt inhibitor (CCT036477, etc.), a Bcl-2 inhibitor, a PD-L1 inhibitor, an ENPP1 inhibitor, or an IDO inhibitor. Thus, the modified MOF can act as a multi-agent delivery platform for a combination of agents with varying solubilities.

In some embodiments, the presently disclosed subject matter provides a nanoparticle (e.g., an MOF nanoparticle (nMOF)) or a nanoparticle formulation for treating cancer. The nanoparticle (e.g., the nMOF) can comprise a metal-containing secondary building unit (e.g., a metal oxo cluster, such as an Hf-metal oxo cluster) and an organic bridging ligand. Attached to the surface of the nMOF can be one or more macromolecules, such as αCD47 or CpG ODNs, and/or one or more small molecule therapeutic agents to activate the immune system against tumors. Additional therapeutic agents (e.g., additional chemotherapeutic or immunotherapy agents), e.g., IMD, can be loaded in the pores and/or cavities of the MOF.

For instance, as described in the Examples below, exemplary IMD-nMOF-DBP-CD47 nanoparticles were prepared. In a colorectal cancer tumor model, treatment with IMD-nMOF-DBP-CD47 intratumorally, followed by low grade X-ray irradiation, provided enhanced tumor regression over CD47, imiquimod, or nMOF-DBP treatment alone. Accordingly, in some embodiments, the presently disclosed subject matter provides improved treatment of tumors using lower doses of CD47 and imiquimod, which can be toxic when delivered at current systemic doses. In some embodiments, MOFs can deliver peptides (e.g., mucin-1 peptide) or a STING activator/agonist, such as cyclic guanosine monophosphate-adenosine monophosphate (cGAMP) or a CpG oligodeoxynucleotide (ODN).

In some embodiments, the presently disclosed subject matter relates to MOFs (e.g., nMOFs) as locally activable immunotherapeutics to release danger-associated molecular patterns (DAMPs) and tumor antigens and to deliver pathogen-associated molecular patterns (PAMPs) for in situ personalized cancer vaccination. As described in the examples below, when activated by X-rays, cationic nMOFs can effectively generate reactive oxygen species to release DAMPs and tumor antigens while delivering anionic CpG ODNs as PAMPs to facilitate the maturation of antigen presentation cells.

In some embodiments, the presently disclosed subject matter provides a MOF (e.g., an nMOF, such as a MOF nanoparticle or MOL) having a surface modified to coordinatively or electrostatically bind to one or more therapeutic agents of interest. In some embodiments, the MOF comprises: (a) a plurality of metal oxo cluster secondary building units (SBUs), wherein each of said metal oxo cluster SBUs comprises one or more first metal ions and one or more anions, wherein each of said one or more anions is coordinated to one or more of the one or more first metal ions; and (b) a plurality of organic bridging ligands linking together the plurality of SBUs to form a two- or three-dimensional matrix. In some embodiments, a plurality of SBUs at a surface of the MOF each comprise a weakly coordinating anion as a SBU capping group anion. Alternatively, in some embodiments, the plurality of organic bridging ligands comprise an organic bridging ligand comprising an electron-withdrawing group or ligand, a positive charge, or a combination thereof. In some embodiments, the plurality of organic bridging ligands comprise a ligand comprising a nitrogen donor group coordinatively bound to a second metal ion, wherein said second metal ion is further coordinated to at least one second metal ligand comprising one or more electron-withdrawing groups. The presently disclosed modified MOF can have a surface that has enhanced (i.e., increased) ability to coordinatively or electrostatically bind to one or more therapeutic agents of interest, e.g., as compared to the surface of an MOF that does not comprise the weakly coordinating capping group or the organic bridging ligand comprising an electron-withdrawing group or ligand. For example, the MOF can have a greater number of sites on its surface that can bond via coordinative or electrostatic interactions with therapeutic agents.

In some embodiments, one or more first metal ion is a metal that absorbs ionizing radiation. In some embodiments, the ionizing radiation absorbable by the metal ion is an X-ray. In some embodiments, one or more of the one or more first metal ions are ions of elements selected from the group including, but not limited to, Hf, a lanthanide metal (La, Ce, Pr, Nd, Pm, Sm, Eu, Gd, Tb, Dy, Ho, Er, Tm, Yb, or Lu), Ba, Ta, W, Re, Os, Ir, Pt, Au, Pb, and Bi. In some embodiments, the MOF is free of Bi and/or W ions. In some embodiments, the first metal ion is an Hf ion. In some embodiments, the plurality of SBUs are metal oxo clusters, e.g., Hf-oxo clusters. In some embodiments, the oxo clusters comprise anions selected from oxide (O²⁻), hydroxide (OH⁻), S²⁻, SH⁻, and formate (HCO₂⁻).

In some embodiments, a plurality of SBUs at a surface of the MOF each comprise a weakly coordinating anion as a capping group. By “capping group” is meant a metal ligand that only coordinates to one metal ion in an SBU. In some embodiments, the capping group is a metal ligand in a MOF SBU that is located at a surface of the MOF, e.g. the upper or lower surface of a MOL or the outer surface of MOF nanoparticle. In some embodiments, the weakly coordinating anion is selected from the group comprising trifluoroacetate and triflate. These weakly coordinating anions can be introduced by modification of a MOF comprising a more strongly coordinating capping group, such as acetate, formate, or benzoate. In some embodiments, the MOF comprises a Hf₁₂ oxo cluster or a Hf₆ oxo cluster.

Each SBU is bonded to at least one other SBU via coordinative bonding to the same bridging ligand. Stated another way, each SBU is bonded via a coordinative bond to at least one bridging ligand, which is also coordinatively bonded to at least one other SBU.

Any suitable bridging ligand or ligands can be used. In some embodiments, each bridging ligand is an organic compound comprising multiple coordination sites. The coordination sites can each comprise a group capable of forming a coordinate bond with a metal cation or a group capable of forming such a group. Thus, each coordination site can comprise an unshared electron pair, a negative charge, or an atom or functional group capable of forming an unshared electron pair or negative charge. Typical coordination sites include, but are not limited to functional groups such as carboxylate and derivatives there (e.g., esters, amides, anhydrides), nitrogen-containing groups (e.g., amines, nitrogen-containing aromatic and non-aromatic heterocycles), alcohols, phenols and other hydroxyl-substituted aromatic groups; ethers, phosphonates, phosphates, thiols, and the like.

In some embodiments, each bridging ligand comprises between 2 and 10 coordination sites (i.e., 2, 3, 4, 5, 6, 7, 8, 9, or 10 coordination sites). In some embodiments, each bridging ligand is capable of binding to two or three SBUs. In some embodiments, each of the organic bridging ligand is a dicarboxylate or a tricarboxylate. For example, the organic bridging ligand can be a porphyrin, chlorin or bacteriochlorin substituted by two carboxylate groups or by two substituents that each comprise a carboxylate group. In some embodiments, each of the two carboxylate groups can form a coordinate bond to the metal ion of two separate SBUs, while the porphyrin, chlorin, or bacteriochlorin nitrogen atoms can form coordinate bonds to another cation or cations (e.g., another metal cation).

In some embodiments, each organic bridging ligand comprises at least two groups wherein each of said two groups is individually selected from the group comprising a carboxylate, an aromatic or non-aromatic nitrogen-containing group (e.g., pyridine, piperidine, indole, acridine, quinolone, pyrrole, pyrrolidine, imidazole, pyrimidine, pyridazine, pyrazine, a triazole, and oxazole), a phenol, an acetylacetonate (acac), a phosphonate, and a phosphate. In some embodiments, at least one bridging ligand is a carboxylate-containing ligand, a pyridine-containing bridging ligand, a phenol-containing ligand, an acetylacetonate-containing bridging ligand, a phosphonate-containing bridging ligand, or a phosphate-containing bridging ligand. In some embodiments, at least one bridging ligand comprises at least two carboxylate groups.

In some embodiments, the plurality of organic bridging ligands comprise a porphyrin substituted by at least two carboxylate groups, optionally wherein the plurality of organic bridging ligands comprise 5,15-di(p-benzoato)porphyrin (DBP).

In some embodiments, each bridging ligand is an organic compound comprising multiple coordination sites, wherein the multiple coordination sites are essentially co-planar or are capable of forming coordinative bonds that are coplanar.

Exemplary organic bridging ligands include, but are not limited to,

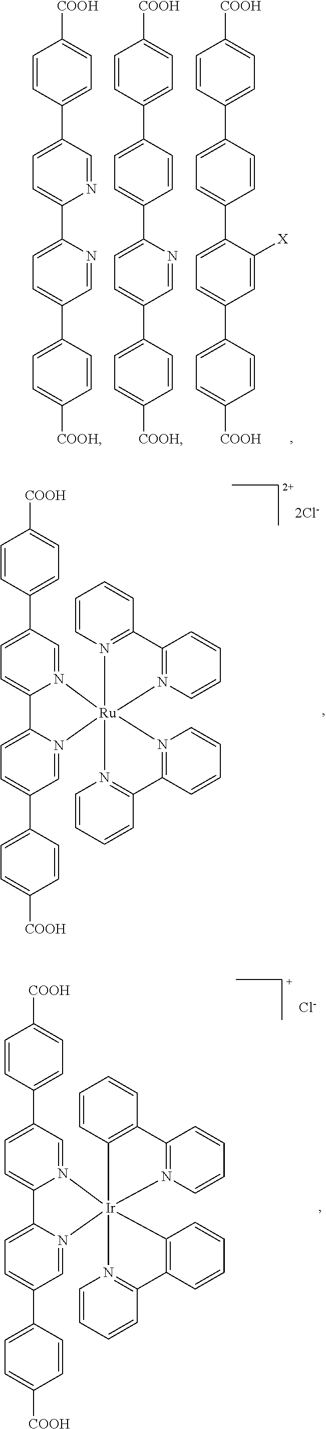

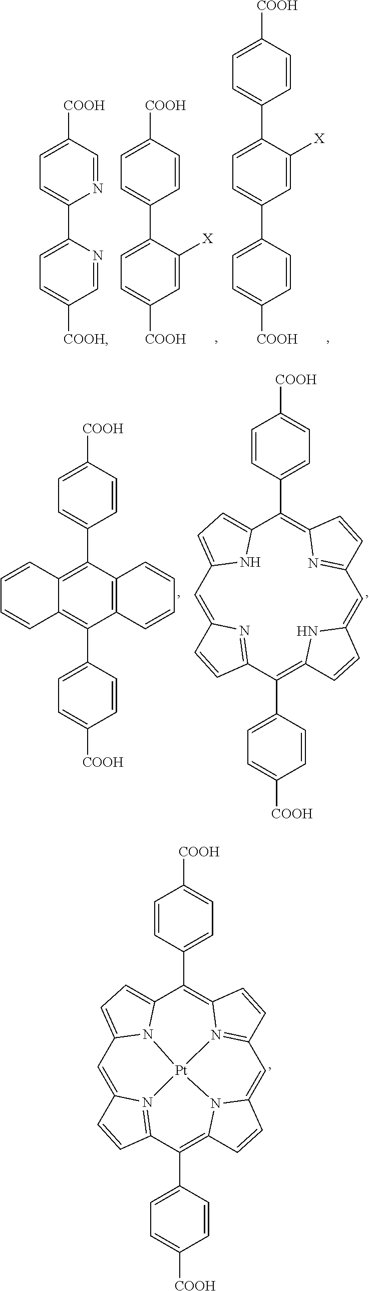

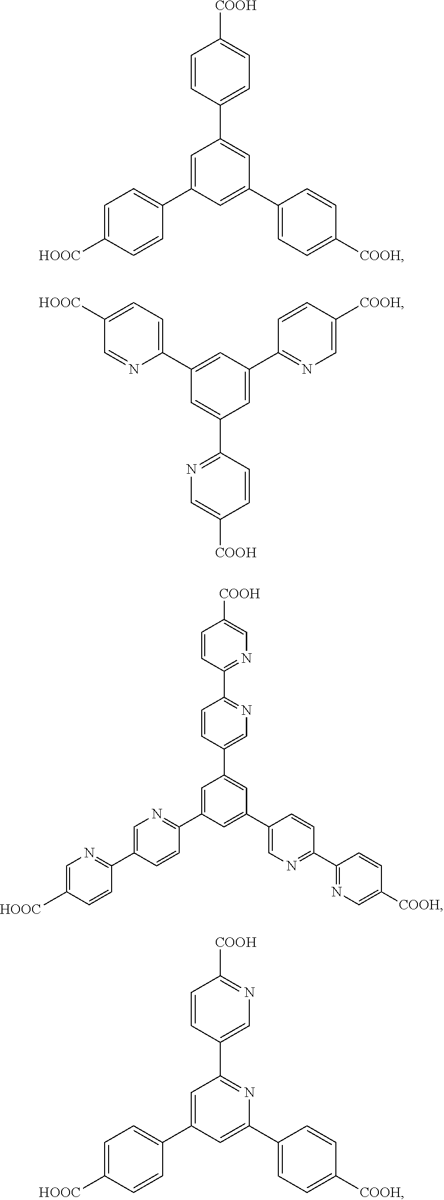

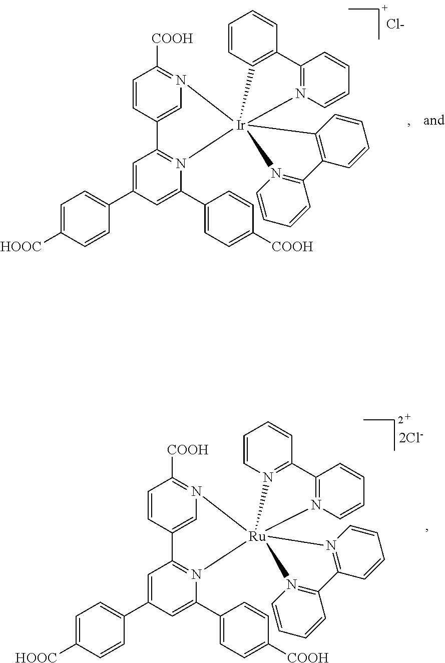

where X, if present, is selected from H, halo (e.g., Cl, Br, or I), OH, SH, NH₂, nitro (NO₂), alkyl, substituted alkyl (e.g., hydroxy-substituted alkyl, thiol-substituted alkyl, or amino-substituted alkyl) and the like. In some embodiments, X comprises a covalently attached photosensitizer such as, but not limited to, a dye, a porphyrin, a chlorin, a bacteriochlorin, a porphycene, or a chlorophyll, or a derivative or analog thereof. For example, X can be a porphyrin covalently attached to the bridging ligand via an alkylene linker moiety and an amide, ester, thiourea, disulfide, or ether bond.

In some embodiments, the linking group of the organic bridging ligand comprises a nitrogen donor moiety. In some embodiments, the organic bridging ligand can comprise a nitrogen donor moiety selected from the group comprising, but not limited to, a bipyridine, a phenyl-pyridine, a phenanthroline, and a terpyridine, which can optionally be substituted with one or more aryl group substituent at one or more of the carbon atoms of the nitrogen donor moiety. In some embodiments, at least one of the organic bridging ligands comprises a ligand selected from the group consisting of 4,4′-di(4-benzoato)-2,2′bipyridine (DBB), 4′,6′-dibenzoato-[2,2′-bipyridine]-4-carboxylate (BPY), and 4′-(4-carboxyphenyl)-[2,2′:6′,2″-terpyridine]-5,5″-dicarboxylate (TPY). In some embodiments, at least one of the organic bridging ligands comprises DBB.

In some embodiments, the MOF further comprises a small molecule therapeutic agent (e.g., a small molecule chemotherapeutic agent) sequestered in pores and/or cavities of the two- or three-dimensional MOF network. In some embodiments, the small molecule therapeutic agent is a chemotherapeutic agent, a small molecule inhibitor and/or a small molecule immunomodulator. In some embodiments, the small molecule therapeutic agent is a small molecule chemotherapeutic agent such as, but not limited to, cisplatin, carboplatin, paclitaxel, SN-35, and etoposide. In some embodiments, the small molecule therapeutic agent is a small molecule inhibitor, such as, but not limited to, a polo-like kinase 1 (PLK1) inhibitor, a Wnt inhibitor, a B-cell lymphoma 2 protein (Bcl-2) inhibitor, a PD-L1 inhibitor, an ectonucleotide pyrophosphatase/phosphodiesterase 1 (ENPP1) inhibitor and an indoleamine 2,3-dioxygenase (IDO) inhibitor. In some embodiments, the IDO inhibitor is selected from the group including, but not limited to indoximod (i.e., 1-methyl-D-tryptophan), BMS-986205, epacadostat (i.e., ICBN24360), and 1-methyl-L-tryptophan. In some embodiments, the small molecule therapeutic agent is a small molecule immunomodulator. In some embodiments, the small molecule immunomodulator is imiquimod (IMD).

In some embodiments, the plurality of organic bridging ligands comprise an organic bridging ligand comprising a nitrogen donor group, wherein said nitrogen donor group is coordinated to a second metal ion and wherein said second metal ion is further coordinated to at least one second metal ligand comprising one or more electron-withdrawing groups, optionally wherein the one or more electron withdrawing groups are halo or haloalkyl (e.g., perhaloalkyl) groups. For example, in some embodiments, the organic bridging ligand comprising a nitrogen donor group comprises a pyridine or bipyridine moiety. In some embodiments, the organic bridging ligand comprising a nitrogen donor group is 4,4′-di(p-benzoato)-2,2′-bipyridine (DBB). In some embodiments, the second metal ion is an iridium (Ir) or a ruthenium (Ru) ion. In some embodiments, the second metal ion is an Ir ion. In some embodiments, the second metal ion is coordinated to two second metal ligands wherein one or both of the second meal ligands comprise one or more electron withdrawing groups, such as, but not limited to, halo, carbonyl, sulfonyl, cyano, nitro, and haloalkyl (e.g., perhaloalkyl). In some embodiments, the haloalkyl group is a halo-substituted methyl group. In some embodiments, the haloalkyl is a perhaloalkyl group (i.e., wherein all of the hydrogen atoms of the alkyl group are replaced by halo). In some embodiments, one or both of the second metal ligands is a bipyridine (bpy) or phenylpyridine (ppy) substituted with one or more electron withdrawing groups. In some embodiments, the bpy or ppy is substituted with at least two groups selected from halo and haloalkyl. In some embodiments the bpy or ppy is substituted by at least two or three groups selected from fluoro (F) and trifluoromethyl. In some embodiments, one or both of the second metal ligands is 2-(2,4-difluorophenyl)-5-(trifluomethyl)pyridine (dF(CF₃)ppy).

In some embodiments, the MOF has a zeta (ζ)-potential value of at least about 5 millivolts (mV) (e.g., at least about 5 mV, at least about 10 mV, at least about 15 mV, at least about 20 mV, or at least about 25 mV. In some embodiments, the MOF has a ζ-potential value of at least about 30 mV.

In some embodiments, the MOF comprises a three-dimensional network. In some embodiments, the three-dimensional network is provided in the form of a nanoparticle. In some embodiments, the MOF comprises a two-dimensional network. In some embodiments, the MOF comprises a nanoscale MOL.

### III.B. Metal-Organic Frameworks with Surface-Bonded Therapeutic Agents

In some embodiments, the presently disclosed subject matter provides a MOF (e.g., a MOF nanoparticle or a MOL) for the delivery of one or more therapeutic agents of interest. In some embodiments, one or more of the one or more therapeutic agents of interest is attached to a surface of the MOF via coordinative bonds or electrostatic interactions. In some embodiments, the MOF comprises: (a) a plurality of metal oxo cluster secondary building units (SBUs), wherein each of said metal oxo cluster SBUs comprises one or more first metal ions and one or more anions, wherein each of said anions is coordinated to one or more of the one or more first metal ions; (b) a plurality of organic bridging ligands linking together the plurality of SBUs to form a two- or three-dimensional matrix; and (c) one or more therapeutic agents of interest bonded to a surface of said MOF via coordinative bonds or electrostatic interactions, optionally wherein one or more therapeutic agents of interest are coordinatively bonded to a metal ion of one or more of the plurality of SBUs at the surface of the MOF.

In some embodiments, the first metal ion is an ion of a metal that absorbs ionizing radiation. Metals that absorb ionizing radiation include high Z-metals (i.e., elements where Z (i.e., the atomic number or proton number) is greater than 40). The ionizing radiation energy can include, for example, X-ray, gamma (γ)-ray, beta (β)-radiation, or proton radiation. In some embodiments, the first metal ion is an ion of a metal that absorbs X-rays. In some embodiments, the first metal ion is an ion of a metal selected from Hf, a lanthanide metal, Ba, Ta, W, Re, Os, Ir, Pt, Au, Pb, and Bi. In some embodiments, the first metal ion is a Hf ion.

Any suitable therapeutic agent of interest can be bonded to the surface. In some embodiments, the one or more therapeutic agents of interest are selected from nucleic acids, small molecule therapeutic agents that comprise a phosphate or carboxylate group, and macromolecules that comprise a surface accessible phosphate or carboxylate group. In some embodiments, the therapeutic agent is a therapeutic agent that targets the immune system, such as one of the agents described hereinabove. In some embodiments, the macromolecule is a protein or peptide. In some embodiments, the protein is an antibody or antibody fragment (e.g., an antibody fragment that includes an antigen binding region). In some embodiments, the protein is selected from an antibody such as, but not limited to, an anti-cluster of differentiation 37 (CD37) antibody, an anti-cluster of differentiation 44 (CD44) antibody, an anti-cluster of differentiation 47 (CD47) antibody, an anti-cluster of differentiation 73 (CD73) antibody, an anti-PD-1 antibody, an anti-PD-L1 antibody, an anti-lymphocyte-activation gene 3 (LAG3) antibody, and an anti-cytotoxic T-lymphocyte-associated protein 4 (CTLA-4) antibody. In some embodiments, the protein is an anti-PD-1 antibody or an anti-PD-L1 antibody.

In some embodiments, the one of the one or more therapeutic agents of interest is a peptide. In some embodiments, the peptide is a peptide targeting membrane mucin-1 (MUC-1) mucins. In some embodiments, the peptide has a cysteine-glutamine-cysteine (CQC) motif. In some embodiments, the peptide is a D-amino acid sequence. In some embodiments, the D-amino acid sequence comprises or consists of amino acid sequence d-CQCRRKN (SEQ ID NO: 1). In some embodiments, the peptide is a membrane-penetrating peptide. In some embodiments, the peptide comprises or consists of the amino acid sequence RRRRRRRRRCQCRRKN (SEQ ID NO: 2).

In some embodiments, one of the one or more therapeutic agents of interest comprises or consists of a nucleic acid. For example, the nucleic acid can be a DNA, a RNA, a miRNA, a mRNA, a siRNA, a ODN, or a cyclic di-nucleotide. In some embodiments, the ODN is a CpG ODN (i.e., a short, single-stranded DNA comprising a cytosine followed by a guanine). In some embodiments, the cyclic di-nucleotide is a STING agonist, such as, but not limited to c-di-AMP or cGAMP.

In some embodiments, the MOF further comprises one or more additional therapeutic agents sequestered in pores or cavities of the two- or three-dimensional network (e.g., in pores in the core of the MOF nanoparticle). In some embodiments, the MOF comprises between about 1 wt % and about 50 wt % (e.g., about 1 wt %, about 5 wt %, about 10 wt %, about 15 wt %, about 20 wt %, about 25 wt %, about 30 wt %, about 35 wt %, about 40 wt %, about 45 wt %, or about 50 wt %) of said one or more additional therapeutic agents. In some embodiments, the one or more additional therapeutic agents can be selected from small molecule chemotherapeutic agents, a small molecule inhibitors and small molecule immunomodulators. Small molecule chemotherapeutic agents include, but are not limited to cisplatin, carboplatin, paclitaxel, SN-35, and etoposide. In some embodiments, the small molecule inhibitors can be selected from PLK1 inhibitors, Wnt inhibitors, Bcl-1 inhibitors, PD-L1 inhibitors, ENPP1 inhibitors and IDO inhibitors. In some embodiments, the additional therapeutic agents include a small molecule immunomodulator. In some embodiments, the small molecule immunomodulator is IMD.

In some embodiments, the plurality of SBUs comprise Hf oxo clusters and the plurality of organic bridging ligands comprise DBP. Thus, in some embodiments, the MOF comprises a Hf-DBP nanoparticle or MOL wherein one or more therapeutic agents of interest are bonded to the surface of said MOF via coordinative bonds to Hf ions of surface accessible SBUs. In some embodiments, the one or more therapeutic agents of interest comprise one or more antibodies. In some embodiments, the one or more therapeutic agents comprise an anti-CD47 antibody. In some embodiments, the MOF further comprises IMD sequestered in pores or cavities of the two- or three-dimensional network of the MOF.

In some embodiments, the MOF is a three-dimensional network and is provided as a nanoparticle. In some embodiments, the MOF comprises about 1 wt % to about 50 wt % of the IMD or the anti-CD47 antibody. In some embodiments, the MOF comprises about 1 wt % to about 25 wt % of the IDM or the anti-CD47 antibody. In some embodiments, the MOF comprises about 9 wt % IMD and about 7.5 wt % anti-CD47 antibody.

In some embodiments, the plurality of SBUs comprise Hf oxo clusters, the plurality of organic bridging ligands comprise DBB coordinated to a second metal ion (e.g., a Ir or Ru ion), wherein said second metal ion (e.g., the Ir or Ru ion) is further coordinated to two (dF(CF₃)ppy); and wherein the one or more therapeutic agents of interest are bonded to the surface of said MOF via electrostatic interactions. In some embodiments, the one or more therapeutic agents of interest comprise a nucleic acid. In some embodiments, the nucleic acid is a STING agonist or a CpG ODN. In some embodiments, the nucleic acid is a CpG ODN.

In some embodiments, the MOF comprises about 1 wt % to about 50 wt % of the one or more therapeutic agents of interest (e.g., about 1, about 5, about 10, about 15, about 20, about 25, about 30, about 35, about 40, about 45, or about 50 wt % of the one or more therapeutic agents of interest). In some embodiments, the MOF comprises about 1 wt % to about 25 wt % of the one or more therapeutic agents of interest. In some embodiments, the one or more therapeutic agents of interest comprise an antibody.

In some embodiments, the plurality of SBUs comprise a Hf oxo cluster, the plurality of organic bridging ligands comprises DBP, wherein the nitrogen atoms of the DBP are coordinated to a metal ion (e.g., a Pt ion), and one or more therapeutic agents of interest are bonded to a surface of the MOF. In some embodiments, the MOF comprises a nanoparticle. In some embodiments, the one or more agents of interest are selected from one or more MUC-1 peptides (i.e., one or more peptides that target MUC-1), a CpG ODN, and cGAMP. In some embodiments, the one or more SBUs comprise a Hf oxo cluster, the one or more organic bridging ligands comprise Ir(DBB)[dF(CF₃)-ppy]₂⁺ (i.e., DBB^{F}-Ir) and cGAMP is bonded to a surface of the MOF. In some embodiments, the MOF is a MOL.

### III.C. Pharmaceutical Formulations

In some embodiments, the presently disclosed subject matter provides a pharmaceutical composition or formulation comprising (i) a MOF as described herein above comprising one or more therapeutic agents of interest bonded to a surface of the MOF and (ii) a pharmaceutically acceptable carrier, e.g., a pharmaceutically acceptable carrier that is pharmaceutically acceptable in humans. In some embodiments, the composition can also include other components, such as, but not limited to lipids, anti-oxidants, buffers, bacteriostatics, bactericidal antibiotics, suspending agents, thickening agents, and solutes that render the composition isotonic with the bodily fluids of a subject to whom the composition is to be administered. In some embodiments, the pharmaceutical composition or formulation further includes an additional therapeutic agent, such as a conventional chemotherapeutic agent or an immunotherapy agent. For example, in some embodiments, the pharmaceutical composition or formulation further includes an antibody immunotherapy agent (e.g., an antibody immune checkpoint inhibitor, such as, but not limited to, an anti PD-1/PD-L1 antibody, an anti-CTLA-4 antibody, an anti-OX40 antibody (i.e., an antibody against cluster of differentiation 134 (CD134), also known as tumor necrosis factor receptor superfamily, member 4 (TNFRSF4)), am anti-T-cell immunoglobulin and mucin domain-containing-3 (TIM3) antibody, an anti-LAG3 antibody, and an anti-CD47 antibody.

The compositions of the presently disclosed subject matter comprise, in some embodiments, a composition that includes a pharmaceutically acceptable carrier. Any suitable pharmaceutical formulation can be used to prepare the compositions for administration to a subject. In some embodiments, the composition and/or carriers can be pharmaceutically acceptable in humans.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the subject; and aqueous and non-aqueous sterile suspensions that can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are sodium dodecyl sulfate (SDS), in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS).

It should be understood that in addition to the ingredients particularly mentioned above, the formulations of this presently disclosed subject matter can include other agents conventional in the art having regard to the type of formulation in question. For example, sterile pyrogen-free aqueous and non-aqueous solutions can be used.

### IV. Methods of Treatment

In some embodiments, the presently disclosed subject matter provides a method of treating cancer in a subject in need thereof. In some embodiments, the method comprises administering to said subject a MOF as described hereinabove where the MOF comprises a plurality of metal oxo cluster SBUs wherein each metal oxo cluster SBU comprises one or more first metal ions and one or more anions, wherein each of said anions is coordinated to one or more of the one or more first metal ions; a plurality of organic bridging ligands linking together the plurality of SBUs to form a two- or three-dimensional network; and one or more therapeutic agents or interest bonded to a surface of said MOF via coordinative bonds or electrostatic interactions (e.g., to a metal ion of one or more of the plurality of SBUs at the surface of the MOF). In some embodiments, the method further comprises exposing the subject (i.e., at least a portion of the subject) to ionizing radiation energy (e.g., X-ray, γ-ray, β-radiation, or proton radiation). For example, after waiting for a period of time (e.g., a few minutes up to a few hours) after administration of the MOF to allow the MOF to localize at a tumor local, the subject can be exposed to ionizing radiation energy. The period of time can be adjusted based on the method of administration of the MOF. In some embodiments, the ionizing radiation energy is X rays. In some embodiments, the MOF is administered directly to a tumor or intravenously. In some embodiments, a portion of the subject's anatomy affected by the cancer or near a site affected by the cancer is exposed to the ionizing radiation.

The subject can be exposed to the ionizing radiation energy in any suitable manner and/or using any suitable equipment, such as that currently being used for delivering X-rays in a medical or veterinary setting. In some embodiments, the X-ray source and/or output can be refined to enhance disease treatment. For instance, the X-rays can be generated using a peak voltage, current and/or, optionally, a filter chosen to minimize DNA damage in the patient due to X-ray radiation and maximize X-ray absorption by the scintillator.

In some embodiments, the subjects are irradiated with a linear accelerator (LINAC), using conventional techniques, Intensity-Modulated Radiation Therapy (IMRT), Image Guided Radiation Therapy (IGRT), or Stereotactic Body Radio Therapy (SBRT), a ⁶⁰Co radiation source, an implanted radioactive seed such as the ones used in brachytherapy, an orthovoltage or supervoltage X-ray irradiator, a high energy electron beam generated from LINAC, or a proton source. In some embodiments, the irradiating can comprise generating X-rays using a tungsten or another metal target, Cobalt-60 sources (cobalt unit), linear accelerators (linacs), Ir-192 sources, and Cesium-137 sources. In some embodiments, the irradiating comprises passing the X-rays (e.g., the X-rays generated using a tungsten target) or other ionizing radiation through a filter prior to irradiation of the subject. In some embodiments, the filter can comprise an element with an atomic number of at least 20. In some embodiments, the filter comprises copper (Cu). In some embodiments, the filter can have a thickness that is less than about 5 millimeters (mm). In some embodiments, the filter can have a thickness of less than about 4 mm (e.g., less than about 3 mm, less than out 1 mm, less than about 0.5 mm, less than about 0.4 mm, less than about 0.3 mm, less than about 0.2 mm, or less than about 0.1 mm).

The X-rays can be generated using a peak voltage, current and/or, optionally, a filter chosen to minimize DNA damage in the patient due to X-ray radiation and maximize X-ray absorption by the scintillator. In some embodiments, the X-rays are generated using a peak voltage that is less than about 230 kVp. In some embodiments, the peak voltage is less than about 225 kVp, less than about 200 kVp, less than about 180 kVp, less than about 160 kVp, less than about 140 kVp, less than about 120 kVp, less than about 100 kVp, or less than about 80 kVp. In some embodiments, the X-rays are generated using a peak voltage that is about 120 kVp.

In some embodiments, X-rays are generated by placing radioactive sources inside the subject on a temporary or permanent basis. In some embodiments, a MOF of the presently disclosed subject matter is injected along with the implantation of a radioactive source.

In some embodiments of the presently disclosed subject matter, the X-ray (or other ionizing radiation energy) source can be refined to enhance the RT-RDT effects of the MOF to enable more efficient cancer cell killing. In some embodiments, the X-ray irradiator can include a panoramic irradiator comprising at least one X-ray source inside a shielded enclosure, the one or more sources each operable to emit X-ray flux across an area equal to the proximate facing surface area of the tumor. See U.S. Patent Application Publication No. 2010/0189222 and WO 2011/049743, each of which is incorporated by reference herein in its entirety. An X-ray generator based on a tungsten target emission is suited for this application. The output energy typically ranges from 100 to 500 kV. In certain embodiments, at least one removable attenuator or filter of selected materials, which contains at least one metal with atomic number >20, is involved in this application. Each attenuator could be a flat board or a board with gradient thickness. See U.S. Pat. No. 7,430,282 incorporated by reference herein in its entirety. The attenuator could be also modulated with periodically spaced grids/holes. The output X-ray energy can be adjusted after filtration by the attenuator to maximize the energy absorption of radiosensitizers/radioscintillators in this application. An X-ray bandpass filter with an x-ray refractive lens for refracting x-rays can also be used. See WO2008/102632, incorporated by reference herein in its entirety.

In some embodiments, the method further comprises administering to the subject an additional therapeutic agent or treatment, such as an immunotherapy agent and/or a cancer treatment. In some embodiments, the additional therapeutic agent or treatment is selected from the group comprising surgery, chemotherapy, toxin therapy, cryotherapy, and gene therapy. The additional cancer treatment can be selected on the basis of the cancer being treated and/or on other factors, such as the patient's treatment history, overall health, etc., in accordance with the best judgement of the treating physician.

In some embodiments, the additional cancer treatment can comprise administering to the patient a conventional chemotherapeutic, such as, but not limited to, a platinum-containing agent (e.g., cisplatin or oxaliplatin or a prodrug thereof), doxorubicin, daunorubicin, docetaxel, mitoxanthrone, paclitaxel, digitoxin, digoxin, and septacidin or another conventional chemotherapeutic known in the art. The additional chemotherapeutic agent can be present in the MOF (e.g., encapsulated or coordinatively or covalently bonded to the MOF). Alternatively, the additional chemotherapeutic agent can be present in the same pharmaceutical composition or formulation as the MOF or in a separate pharmaceutical composition or formulation, administered prior to, simultaneously with, or after administration of the pharmaceutical composition or formulation comprising the MOF and/or the radiation.

In some embodiments, the additional cancer treatment can involve administering to the patient a drug formulation selected from the group comprising a polymeric micelle formulation, an asymmetric lipid bilayer, a liposomal formulation, a dendrimer formulation, a polymer-based nanoparticle formulation, a silica-based nanoparticle formulation, a nanoscale coordination polymer formulation, a nanoscale metal-organic framework formulation, and an inorganic nanoparticle (gold, iron oxide nanoparticles, etc.) formulation. In some embodiments, the drug formulation can be a formulation including a conventional chemotherapeutic.

The immunotherapy agent for use according to the presently disclosed subject matter can be any suitable immunotherapy agent known in the art. Immunotherapeutic agents suitable for use in the presently disclosed subject matter include, but are not limited to: PD-1, PD-L1, CTLA-4, IDO and CCR7 inhibitors, that is, a composition that inhibits or modifies the function, transcription, transcription stability, translation, modification, localization, or secretion of a polynucleotide or polypeptide encoding the target or a target associated ligand, such as anti-target antibody, a small molecule antagonist of the target, a peptide that blocks the target, a blocking fusion protein of the target, or small-interfering ribonucleic acid (siRNA)/shRNA/microRNA/pDNA suppressing the target. Antibodies that can be used according to the presently disclosed subject matter include, but are not limited, to: anti-CD52 (Alemtuzumab), anti-CD20 (Ofatumumab), anti-CD20 (Rituximab), anti-CD47 antibodies, anti-GD2 antibodies, etc. Conjugated monoclonal antibodies for use according to the presently disclosed subject matter include but are not limited to: radiolabeled antibodies (e.g., Ibritumomab tiuxetan (Zevalin), etc.), chemolabeled antibodies (antibody-drug conjugates (ADCs)), (e.g., Brentuximab vedotin (Adcetris), Ado-trastuzumab emtansine (Kadcyla), denileukin diftitox (Ontak) etc.). Cytokines for use according to the presently disclosed subject matter include, but are not limited to: interferons (i.e., IFN-α, INF-γ), interleukins (i.e. IL-2, IL-12), TNF-α, etc. Other immunotherapeutic agents for use according to the presently disclosed subject matter include, but are not limited to, polysaccharide-K, neoantigens, etc.

In some embodiments, the immunotherapy agent can be selected from an agonist of DNA or RNA sensors, such as a RIG-I agonist (e.g., a compound described in U.S. Pat. No. 7,271,156, incorporated herein by reference in its entirety), a TLR3 agonist (e.g., polyinosinic:polycytidylic acid), a TLR7 agonist (e.g., IMD), a TLR9 agonist (e.g., CpG ODN), and a STING agonist (e.g., STINGVAX or ADU-S100). In some embodiments, the immunotherapy agent is selected from the group comprising a PD-1 inhibitor (e.g., pembrolizumab or nivolumab), a PD-L1 inhibitor (e.g., atezolizumab, avelumab, or durvalumab), a CTLA-4 inhibitor (e.g., ipilimumab), an IDO inhibitor (e.g., indoximod, BMS-986205, or epacadostat), and a CCR7 inhibitor. In some embodiments, the immunotherapy agent is selected from the group including, but not limited to, an anti-PD-1/PD-L1 antibody, an anti-IDO inhibitor, an anti-CTLA-4 antibody, an anti-OX40 antibody, an anti-TIM3 antibody, an anti-LAG3 antibody, an siRNA targeting PD-1/PD-L1, an siRNA targeting IDO and an siRNA targeting CC chemokine receptor 7 (CCR7), as well as any other immunotherapy agent as recited elsewhere herein or that is known in the art.

Thus, in some embodiments, the presently disclosed subject matter provides a method of treating a cancer that combines X-ray induced RDT and immunotherapy. Accordingly, in some embodiments, the presently disclosed subject matter provides a method comprising: administering to a patient a MOF or MOL as described herein comprising one or more surface bonded therapeutic agents and irradiating at least a portion of the patient with X-rays (e.g., in one to fifty fractions); and administering to the patient an immunotherapy agent. The immunotherapy agent can be administered either simultaneously with a MOF and/or the irradiating, or prior to or after administering the MOF and/or the irradiating. In some embodiments, the immunotherapeutic agent is selected from the group including, but not limited to, an agonist of DNA or RNA sensors, such as a RIG-1 agonist, a Toll-like receptor 3 (TLR3) agonist (e.g., polyinosinic:polycytidylic acid), a Toll-like receptor 7 (TLR7) agonist (such as IMD), a Toll-like receptor 9 (TLR9) agonist (e.g., CpG ODNs), a stimulator of interferon genes (STING) agonist (e.g., STINGVAX or ADU-S100), or an IDO inhibitor. In some embodiments, the IDO inhibitor is selected from the group including, but not limited to indoximod (i.e., 1-methyl-D-tryptophan), BMS-986205, epacadostat (i.e., ICBN24360), and 1-methyl-L-tryptophan. In some embodiments, the immunotherapy agent is an immune checkpoint inhibitor. The immune checkpoint inhibitor can be an antibody, such as an anti-PD-1/PD-L1 antibody (i.e., an anti-PD-1 antibody or an anti-PD-L1 antibody), an anti-CTLA-4 antibody, an anti-OX40 antibody, an anti-TIM3 antibody, an anti-LAG3 antibody, or an anti-CD47 antibody. In some embodiments, the immunotherapy agent is an anti-PD-L1 antibody.

In some embodiments, the cancer is selected from a head tumor, a neck tumor, breast cancer, a gynecological tumor, a brain tumor, colorectal cancer, lung cancer, mesothelioma, a soft tissue sarcoma, skin cancer, connective tissue cancer, adipose cancer, stomach cancer, anogenital cancer, kidney cancer, bladder cancer, colon cancer, prostate cancer, central nervous system cancer, retinal cancer, blood cancer, a neuroblastoma, multiple myeloma, lymphoid cancer, and pancreatic cancer, In some embodiments, the disease is selected from a colorectal cancer, a melanoma, a head and neck cancer, a brain cancer, a breast cancer, a liver cancer, a lung cancer, and a pancreatic cancer. In some embodiments, the disease is selected from a colorectal cancer, a melanoma, a lung cancer, and a pancreatic cancer. In some embodiments, the disease is a metastatic cancer.

In some embodiments, the use of the presently disclosed MOF provides an extended release profile for one or more of the one or more therapeutic agents of interest (e.g., compared to administration of the free, non-MOF bonded therapeutic agent of interest). In some embodiments, the release rate is tunable. In some embodiments, the MOF provides sustained release of one or more therapeutic agents of interest over a period of a few hours or a few days. In some embodiments, administration of the MOF lowers the therapeutically effective dose of the one or more therapeutic agents of interest (e.g., compared to when the same therapeutic agent of interest is administered in free form, non-associated with a MOF). In some embodiments, the sustained release of the therapeutics can be tuned from 4 hours to 2 weeks. In some embodiments, this can be achieved by tuning the numbers of metal open coordination sites and the number of charges on the SBUs and the number of charges and the electronic density of organic ligands and by selecting therapeutic agents of proper charges, coordination strength, and multivalency.

### V. Subjects

The methods and compositions disclosed herein can be used on a sample either in vitro (for example, on isolated cells or tissues) or in vivo in a subject (i.e. living organism, such as a patient). In some embodiments, the subject or patient is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided are methods and compositions for mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), rodents (such as rats, mice, hamsters, guinea pigs, etc.), ruminants (such as cattle, oxen, sheep, giraffes, deer, goats, bison, and camels), and horses. Also provided is the treatment of birds, including the treatment of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment of livestock including, but not limited to domesticated swine (pigs and hogs), ruminants, horses, poultry, and the like.

### VI. Administration

Suitable methods for administration of a composition of the presently disclosed subject matter include, but are not limited to intravenous and intratumoral injection, oral administration, subcutaneous administration, intraperitoneal injection, intracranial injection, and rectal administration. Alternatively, a composition can be deposited at a site in need of treatment in any other manner, for example by spraying a composition within the pulmonary pathways. The particular mode of administering a composition of the presently disclosed subject matter depends on various factors, including the distribution and abundance of cells to be treated and mechanisms for metabolism or removal of the composition from its site of administration. For example, relatively superficial tumors can be injected intratumorally. By contrast, internal tumors can be treated following intravenous injection.

In some embodiments, the method of administration encompasses features for regionalized delivery or accumulation at the site to be treated. In some embodiments, a composition is delivered intratumorally. In some embodiments, selective delivery of a composition to a subject is accomplished by intravenous injection of the composition followed by ionizing radiation treatment (e.g., X-ray radiation) of the subject.

For delivery of compositions to pulmonary pathways, compositions of the presently disclosed subject matter can be formulated as an aerosol or coarse spray. Methods for preparation and administration of aerosol or spray formulations can be found, for example, in U.S. Pat. Nos. 5,858,784; 6,013,638; 6,022,737; and 6,136,295.

### VII. Doses

An effective dose of a composition of the presently disclosed subject matter is administered to a subject. An “effective amount” is an amount of the composition sufficient to produce detectable treatment. Actual dosage levels of constituents of the compositions of the presently disclosed subject matter can be varied so as to administer an amount of the composition that is effective to achieve the desired effect for a particular subject and/or target. The selected dosage level can depend upon the activity (e.g., RT-RDT activity or MOF and/or MOL loading) of the composition and the route of administration.

After review of the disclosure herein of the presently disclosed subject matter, one of ordinary skill in the art can tailor the dosages to an individual subject, taking into account the particular formulation, method of administration to be used with the composition, and nature of the target to be treated. Such adjustments or variations, as well as evaluation of when and how to make such adjustments or variations, are well known to those of ordinary skill in the art.

### VIII. Methods of Modifying Metal-Organic Frameworks

In some embodiments, the presently disclosed subject matter provides a method of enhancing surface interaction and/or bonding of one or more therapeutic agents of interest to a MOF. In some embodiments, the method comprises modifying the surface of an MOF (i.e., an MOF comprising a plurality of metal-oxo cluster SBUs and a plurality of organic bridging ligands linking together the plurality of SBUs) by (i) providing one or more surface accessible coordination sites coordinatively bonded to a weakly coordinated anion that can be replaced by a carboxylate or phosphate substituent of a therapeutic agent of interest or (ii) providing a MOF comprising one or more electron-withdrawing bridging ligands, one or more bridging ligands comprising a positive charge, or a combination thereof.

In some embodiments, the method comprises (ia) providing a parent MOF comprising metal oxo cluster SBUs linked together via organic bridging ligands, wherein each of said SBUs comprises one or more metal ions and one or more anions, and wherein said MOF comprises a plurality of surface accessible metal oxo cluster SBUs where the one or more anions of each of said surface accessible metal oxo cluster SBUs comprise a strongly coordinating anion as a SBU capping group; and (ib) removing the strongly coordinating anion by contacting the parent MOF with a suitable reagent to replace the strongly coordinating anion with a weakly coordinating anion. In some embodiments, the strongly coordinating anion is an acetate or a formate anion. In some embodiments, the reagent is selected from trimethylsilyl trifluoroacetate (TMS-TFA), trimethylsilyl triflate, and a mineral acid having a pKa of less than about 3. In some embodiments, the weakly coordinating anion is a trifluoroacetate anion or a triflate anion.

In some embodiments, the method comprises providing a MOF comprising one or more bridging ligands comprising an electron-withdrawing group, one or more bridging ligands comprising a positive charge, or a combination thereof. In some embodiments, the electron-withdrawing group is selected from halo, carbonyl, sulfonyl, cyano, nitro, and haloalkyl (e.g., perhaloalkyl). In some embodiments, the electron-withdrawing group is fluoro or trifluoromethyl. In some embodiments, the method comprises providing a MOF comprising metal oxo cluster SBUs linked together via organic bridging ligands, wherein each of said SBUs comprise one or more first metal ions (e.g., Hf) and one or more anions coordinated to said one or more first metal ions, and wherein said organic bridging ligands comprise at least one organic bridging ligand comprising a coordinated, non-SBU-associated second metal ion (e.g., Ir, Ru, or Pt), wherein said second metal ion is further coordinated to one or more electron-withdrawing ligand, optionally wherein said electron-withdrawing ligand is a halo and/or perhaloalkyl-substituted bipyridine or phenylpyridine ligand. In some embodiments, the electron-withdrawing ligand is a halo- and/or perhaloalkyl-substituted bipyridine ligand. In some embodiments, providing the MOF comprises providing an MOF comprising a di(4-benzoato)-2,2′-bypyridine (DBB) bridging ligand, wherein said DBB bridging ligand is coordinated to a first metal ion of two different metal oxo cluster SBUs and to a second metal ion and wherein said second metal ion is further coordinated to two halo and/or perhaloalkyl-substituted pyridine ligands, In some embodiments, the halo and/or perhaloalkyl-substituted pyridine ligands are each 2-(2,4-difluorophenyl)-5-(trifluoromethyl)-pyridine. In some embodiments, the second metal ion is Ir. In some embodiments, the MOF comprises one or more SBU comprising a metal ion that absorbs ionizing radiation. In some embodiments, the ionizing radiation is x-rays. In some embodiments, the metal ion is an ion of an element selected from Hf, a lanthanide metal, Ba, Ta, W, Re, Os, Ir, Pt, Au, Pb, and Bi. In some embodiments, the metal ion is not a Bi ion or a W ion. In some embodiments, the metal ion is a Hf ion.

In some embodiments, the MOF has enhanced interaction and/or bonding ability for one or more therapeutic agents of interest compared to a MOF without surface modification. In some embodiments, the one or more therapeutic agents of interest are selected from a nucleic acid, a small molecule, and/or macromolecule comprising a surface accessible phosphate or carboxylate group. In some embodiments, the protein is selected from the group comprising an anti-CD37 antibody, an anti-CD44 antibody, an anti-CD47 antibody, an anti-CD73 antibody, an anti-PD-1 antibody, an anti-PD-L1 antibody, an anti-LAG3 antibody, and an anti-CTLA-4 antibody. In some embodiments, the nucleic acid is selected from the group comprising miRNA, mRNA, siRNA, CpG ODN, and a cyclic di-nucleotide. In some embodiments, the cyclic di-nucleotide is a STING agonist. In some embodiments, the STING agonist is c-di-AMP or cGAMP.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Materials and Methods for Examples 1-8

All chemicals were purchased from Sigma-Aldrich (St. Louis, Missouri, United States of America) and Fisher (Thermo Fisher Scientific, Waltham, Massachusetts, United States of America) unless mentioned otherwise and used without further purification. Imiquimod (powder, 95%) was purchased from Cayman Chemical (Ann Arbor, Michigan, United States of America). InVivoMAb anti-mouse CD47 polyclonal antibody (αCD47, MIAP301) was purchased from Bio X Cell (Lebanon, New Hampshire, United States of America). Nuclear magnetic resonance (NMR) spectra were collected using a 500 MHz NMR spectrometer sold under the tradename Avance II™ (Bruker, Billerica, Massachusetts, United States of America) with default Bruker QNP probe ¹⁹F{¹H} (Bruker, Billerica, Massachusetts, United States of America) and referenced to ¹H resonance from incomplete deuteration of DMSO-D6. Transmission electron microscopy (TEM) was performed with a TECNAI Spirit TEM (FEI Company, Hillsboro, Oregon, United States of America). Ultraviolet-visible (UV-vis) absorption spectra were acquired with a UV-2600 UV-Vis spectrophotometer (Shimadzu, Kyoto, Japan). Particle sizes were collected via dynamic light scattering (DLS) and ζ-potentials were measured by electrophoresis with a Nano Series Zeta-Sizer (Malvern Panalytical, Westborough, Massachusetts, United States of America). Powder X-ray diffraction (PXRD) data was collected on a D8 Venture diffractometer (Bruker, Billerica, Massachusetts, United States of America) using a Cu Kα radiation source (λ=1.54178 Å) and processed with PowderX software. Inductively coupled plasma-mass spectrometry (ICP-MS) data was obtained with a 7700×ICP-MS (Agilent Technologies, Santa Clara, California, United States of America) and analyzed using ICP-MS Mass Hunter version B01.03. Samples were diluted in a 2% HNO₃ matrix and analyzed with an ¹¹⁵In internal standard against a 10-point standard curve over the range of 0.1 ppb to 500 ppb. The correlation was >0.999 for all analyses of interest. Data collection was performed in spectrum mode with five replicates per sample and 100 sweeps per replicate. The LC-MS data was collected on an Agilent 6540 Q-Tof MS-MS spectrometer with 1290 UHPLC (Cis reverse) (Agilent Technologies, Santa Clara, California, United States of America). The mobile phase was 35% MeOH/65% H₂O and the linear range of the imiquimod (IMD) calibration curve was 0 to 200 ng/ml using 10 μL injection volume. Thermogravimetric analysis (TGA) was performed in air using a Shimadzu TGA-50 (Shimadzu, Kyoto, Japan) equipped with a platinum pan and heated at a rate of 1° C. per min. Flow cytometry data was collected on an LSR-Fortessa 4-15 (BD Biosciences, San Jose, California, United States of America) and analyzed by FlowJo software (Tree Star, Ashland, Oregon, United States of America). Confocal laser scanning microscope images were collected on an Olympus FV1000 and analyzed with ImageJ software (NIH, Bethesda, Maryland, United States of America). Concentration of αCD47 was measured by either a spectrophotometer sold under the tradename NANODROP® 8000 (Thermo Fisher Scientific, Waltham, Massachusetts, United States of America) or a protein assay kit sold under the tradename PIERCE™ BCA Protein Assay Kit (Thermo Fisher Scientific, Waltham, Massachusetts, United States of America). ELISpot assay was performed with an Mouse IFN-γ assay sold under the tradename ELISpot READY-SET-GO!™ (eBioscience, San Diego, California, United States of America).

Murine colon adenocarcinoma cell CT26 was obtained from the American Type Culture Collection (ATCC, Manassas, Virginia, United States of America) and were cultured in RPMI 1640 medium (GE Healthcare, Chicago, Illinois, United States of America) with 10% fetal bovine serum, 100 U/ml penicillin G sodium and 100 μg/ml streptomycin sulphate in a humidified atmosphere containing 5% CO₂ at 37° C. BALB/c mice (6-8 weeks) were obtained from Harlan-Envigo Laboratories, Inc (Indianapolis, Indiana, United States of America).

An RT250 orthovoltage X-ray machine model (Philips, Andover, Massachusetts, United States of America) with fixed setting at 250 kVp, 15 mA and a built-in 1 mm Cu filter was used for in vitro assays. An X-RAD 225 image-guided biological irradiator (Precision X-ray Inc., North Branford, Connecticut, United States of America) was used for in vivo studies. The instrument was set at 225 kVp and 13 mA, with a 0.3 mm flat-board Cu filter installed before a 25 mm collimator.

### Example 2

### Synthesis and Characterization of Hf-DBP and TFA-Modified Hf-DBP

Surface modification of Hf-DBP with TFA: 5,15-di(p-benzoato)porphyrin (H₂DBP) and Hf-DBP were synthesized as previously reported (Lu et al., J. Am. Chem. Soc., 2014, 136 (48), 16712-16715). A Hf-DBP suspension in EtOH was washed sequentially with acetonitrile and benzene by sonication and centrifugation to lower the polarity of the solvent. The Hf-DBP in benzene was degassed with N2 and transferred to a glove box for surface modification reaction. In a 1-dram vial with a stir bar, 1 mL of Hf-DBP suspension in benzene (2 mM) and 10-fold trimethylsilyl trifluoroacetate (TFA-TMS) were added and the vial was sealed. The reaction mixture was stirred for 12 hours to obtain TFA-modified Hf-DBP. Outside of the glove box, the suspension was washed with CH₃CN and EtOH sequentially and stored in EtOH for further use.

Digestion and NMR analysis of TFA-modified Hf-DBP: About 1.0 mg Hf-DBP or TFA-modified Hf-DBP was dried under vacuum and re-suspended in a mixture of 500 mL DMSO-D₆ and 50 mL D₃PO₄. The mixture was sonicated for 15 min and an additional 50 mL D20 was added. The resultant mixture was sonicated to afford a homogeneous solution for ¹H and ¹⁹F NMR analyses. ¹H NMR (500 MHz, DMSO-D₆, ppm) for digested Hf-DBP: 6=10.65 (s, 2H), 9.67 (d, 4H), 9.03 (d, 4H), 8.40 (m, 8H). ¹H NMR (500 MHz, DMSO-D6, ppm) for digested TFA-modified Hf-DBP: δ=10.65 (s, 2H), 9.67 (d, 4H), 9.03 (d, 4H), 8.40 (m, 8H) (DBP aryl H), δ=1.89 (s) (HOAc alkyl H). ¹⁹F NMR (471 MHz, DMSO-D6, ppm) for digested TFA modified Hf-DBP: δ=−74.25 (s). In Hf-DBP, the surface modulators were HOAc and gave strong signals δ=1.89 (s, 1.2H) in ¹H NMR spectrum. In Hf-DBP-TFA, δ=1.89 (s, 0.11H) showed significantly reduced signals which indicated the surface modulators OAc were mostly (>90%) exchanged to TFA. δ=−74.25 (s) ¹⁹F NMR also confirmed the existence of TFA on MOF surface.

### Example 3

### Synthesis and Characterization of IMD@Hf-DBP

Synthesis of IMD@Hf-DBP: In a 20 mL glass vial, 10 mg of IMD and 5 mL of 2 mM TFA-modified Hf-DBP in EtOH were added. The mixture was sonicated for 1 hour at 40-50° C. The reaction was stirred at room temperature for another 12 hours, then sequentially washed with DMSO:EtOH=1:1 and EtOH by centrifugation and sonication to remove excess imiquimod. The supernatant of IMD@Hf-DBP was detected by UV-Vis to confirm no free imiquimod was present.

Digestion and UV-Vis analysis of IMD@Hf-DBP: To determine the components and estimate loading percentage of IMD, 5 mL of 2 mM IMD@Hf-DBP was added into 900 mL DMSO and 100 mL H₃PO₄. The mixture was sonicated for 2 hours, then analyzed by UV-Vis spectroscopy.

Digestion and NMR analysis of IMD@Hf-DBP: About 1.0 mg IMD@Hf-DBP was dried under vacuum and re-suspended in a mixture of 500 μL DMSO-D₆ and 50 mL D₃PO₄. The mixture was sonicated for 15 min, and additional 50 mL D₂O was added. The mixture was vortexed to afford a homogeneous solution. The samples were analyzed by ¹H and ¹⁹F NMR. ¹H NMR (500 MHz, DMSO-D₆, ppm): δ=10.65 (s, 2H), 9.67 (d, 4H), 9.03 (d, 4H), 8.40 (m, 8H) (DBP aryl H), δ=1.89 (s) (HOAc alkyl H), δ=8.41 (s, 1H), 8.14 (d, 1H), 7.77 (d, 1H), 7.70 (t, 1H), 7.59 (t, 1H), 4.44 (d, 2H), 2.10 (m, 1H), 0.90 (d, 2H) (IMD H). ¹⁹F NMR (471 MHz, DMSO-D₆, ppm): δ=−74.25 (s) (TFA F). The weight percentage of loaded IMD was calculated to be 8.8% by the integration of δ=0.90.

TGA analysis of IMD@Hf-DBP: About 2 mg of IMD@Hf-DBP was dried under vacuum and used for TGA analysis. The theoretical weight loss was 62.0% and experimental weight loss was 66.2%. The calculated loading percentage by TGA was ˜11.1%.

### Example 4

### Synthesis and Characterization of IMD(@Hf-DBP/αCD47

Synthesis of IMD@Hf-DBP/αCD47: IMD@Hf-DBP was resuspended in 1 mL PBS at an equivalent Hf concentration of 2 mM in a 1.5 mL Eppendorf tube. 750 mg of αCD47 (8.8 mg/mL in PBS, 85.2 mL) was added to the tube and vortexed for 15 seconds. The loading of αCD47 was confirmed by nanodrop which determined the supernatant IgG concentration after centrifugation.

The particle sizes and ζ-potentials of Hf-DBP, TFA-modified Hf-DBP, IMD@Hf-DBP, and IMD@Hf-DBP/αCD47 were measured by in purified water (MILLI-Q® water, Millipore Sigma, Burlington, Massachusetts, United States of America). Results are summarized below in Table 1.

TABLE 1
Particle sizes, PDI, and ζ-potentials

| | Number-average | | |
|---|---|---|---|
| Particles | size | PDI | ζ-potential |
| Hf-DBP | 130.8 ± 7.1 nm | 0.099 | −23.3 ± 1.0 mV |
| TFA modified Hf-DBP | 134.2 ± 12.2 nm | 0.250 | −19.5 ± 0.7 mV |
| IMD@Hf-DBP | 161.2 ± 14.3 nm | 0.131 | −16.1 ± 0.7 mV |
| IMD@Hf-DBP/αCD47 | 212.9 ± 4.2 nm | 0.163 | −4.7 ± 0.5 mV |

Release Profiles of IMD@Hf-DBP/αCD47:

Quantification methods for αCD47: IMD@Hf-DBP/αCD47 was re-suspended in 27 mL PBS at an equivalent Hf concentration of 0.5 mM and aliquoted into 27 Eppendorf tubes (N=3 for each time point). The tubes were transferred to an incubator at 37° C. and measured at 0 h, 1 h, 2 h, 4 h, 8 h, 12 h, 24 h, 36 h and 48 h. At each time point, three Eppendorf tubes were taken out and spun down at 12000 rpm. The supernatants were then collected for LC-MS analysis of imiquimod and BCA assays of αCD47.

Quantification methods of IMD: The standard curve of imiquimod was prepared by dissolving pure imiquimod in PBS (1 ppm stock solution and gradient dilution) and the linear range was between 10 ppb and 500 ppb. The gradient elution of the LC-MS was set as: 1) 0-3 min, 100% H₂O; 2) 3-8 min, 65% H₂O and 35% MeOH; 3) 8-10 min 100% MeOH. The flow rate was 0.5 mL/min with injection volume of 10 mL and the samples were diluted 10-fold for the LC-MS analysis.

Release profiles of IgG-FITC in serum-containing PBS: IMD@Hf-DBP/IgG-FITC was prepared in the same way as IMD@Hf-DBP/αCD47 and re-suspended in 27 mL PBS with 10% FBS at an equivalent Hf concentration of 0.1 mM and aliquoted into 27 Eppendorf tubes (N=3 for each time point). The tubes were transferred to an incubator at 37° C. and measured at 0 h, 1 h, 2 h, 4 h, 8 h, 12 h, 24 h, 36 h and 48 h. At each time point, three Eppendorf tubes were taken out and spun down at 12000 rpm. The supernatants were then collected for fluorescence detection by a microplate reader. The release percentage was calculated based on relative fluorescence fitted into an IgG-FITC standard curve. IgG was released relatively quickly over the first about 10 hours and then started to level off. The release percentage after 12 hours was about 50% and after 48 hours was about 60%

### Example 5

### In Vitro Studies of IMD@Hf-DBP and IMD@Hf-DBP/α-CD47

Cellular uptake of IMD@Hf-DBP and IMD@Hf-DBP/α-CD47: The cellular uptake of IMD@Hf-DBP and IMD@Hf-DBP/α-CD47 was evaluated on CT26 cells on 6-well plates at a density of 2.5×10⁵/ml in RPMI medium. IMD@Hf-DBP and IMD@Hf-DBP/α-CD47 were added to each well at an equivalent Hf concentration of 20 μM in medium and the plates were shake at 150 rpm for 1 min. The cells were then put back into the 37° C. incubator and incubated for 1, 2, 4, and 8 hours. At each time point, the medium was removed, the cells were washed with 2 mL DPBS three times, trypsinized, collected by centrifugation at 3000 rpm and counted by a hemocytometer. The cell pellets were digested with 1 mL 99% concentrated HNO₃ (67-70% trace metal grade) and 1% HF in 1.5 mL ep tubes for 48 hours with strong vortex and sonication every 12 hours. The Hf concentration was then determined by ICP-MS

Cytotoxicity of IMD@Hf-DBP and IMD@Hf-DBP/α-CD47: Dark toxicity of IMD@Hf-DBP and IMD@Hf-DBP/α-CD47 was evaluated on CT26 cells or HEK293T cells with 3-(4,5-dimethylthiazol-2-yl)-5-(3-carboxymethoxyphenyl)-2-(4-sulfo-phenyl)-2H-tetrazolium (MTS) assay (Promega Corporation, Madison, Wisconsin, United States of America, 1/10 dilution in DMEM). The cells were first seeded on 96-well plates at a density of 15000 cells/mL with 100 μL RPMI/DMEM medium per well and further cultured overnight. IMD@Hf-DBP and IMD@Hf-DBP/α-CD47 was added to the wells at an equivalent Hf concentration of 0, 0.5, 1, 2, 5, 10, 20, 50, 100 μM and further incubated for 72 hours before determining the cell viability by MTS assay. In the absence of X-ray irradiation, IMD@Hf-DBP/αCD47 was slightly toxic to CT26 colon cancer cells (see FIG. 12) but showed no toxicity on HEK-293T cells.

Macrophage Activation: BALb/c bone-marrow-derived monocytic cells were harvested, cultured, and activated. For classically activated M1 macrophages, bone-marrow cells were incubated with fresh Dulbecco's Modified Eagle Medium (DMEM) medium supplemented with 20% v/v fetal bovine serum, 100 ng/mL lipopolysaccharides, and 25 ng/mL IFN-γ for 48 h. The adherent cells were harvested for the following studies. For M2 macrophages, 100 ng/mL murine granulocyte-macrophage colony-stimulating granulocyte factor and 20 ng/mL IL-4 were added. Cells were incubated at 37° C. under 5% CO₂. The medium was replaced every 2-3 days, and cells were used between 6 and 8 days of culture.

Macrophage repolarization: CT26 cells were cultured in 6-well plate overnight and incubated with PBS, IMD, Hf-DBP, or IMD@Hf-DBP at an equivalent dose of 20 μM for 4 h followed by X-ray irradiation at a dose of 0 or 2 Gy. Differentiated M2 macrophages were added and co-cultured with the treated CT26 cells at 37° C. for 12 h. Cells were then collected, washed twice with cold PBS, blocked with anti-CD16/32 to reduce nonspecific binding to FcRs, stained with CD86 (GL1), CD206 (C068C2) and analyzed by flow cytometry.

Calreticulin translocation: The immunogenic cell death induced by RT-RDT treatment was investigated by detecting the exposure of calreticulin (CRT) on cell surface. CT26 cells were cultured in 6-well plate overnight and incubated with PBS, αCD47, Hf-DBP, or Hf-DBP/αCD47 at an equivalent dose of 20 μM for 4 h followed by X-ray irradiation at a dose of 0 or 2 Gy. Cells were then cultured in the incubator for another 4 hours to induce CRT translocation, followed by fixation and staining with AlexaFluor 488-CRT antibody (Enzo Life Science, Farmingdale, New York, United States of America) and DAPI on cover slides for confocal imaging or flow cytometric analysis.

Phagocytosis: 5×10⁵ CFSE-labeled CT26 cells were cultured in a 6-well plate overnight and incubated with PBS, αCD47, Hf-DBP or Hf-DBP-αCD47 at an equivalent dose of 20 μM for 4 h, followed by X-ray irradiation at a dose of 0 or 2 Gy. 1.5×10⁶ PerCP-Cy5.5-labeled macrophages were added and co-cultured with the treated CT26 cells at 37° C. for 4 h. Cells were then collected, washed twice with cold PBS, and imaged by CLSM or analyzed by flow cytometry.

### Example 6

### In Vivo Studies of IMD@Hf-DBP and IMD@Hf-DBP/α-CD47

Profiling of innate immunity: Tumors were harvested, treated with 1 mg/ml collagenase I (Gibco Laboratories, Gaithersburg, Maryland, United States of America) for 1 h in a 37° C. water bath, and ground using the rubber end of a syringe. Cells were filtered through nylon mesh filters with size of 70 μm and washed with PBS. The single-cell suspension was incubated with anti-CD16/32 (clone 93; eBiosciences, San Diego, California, United States of America) to reduce nonspecific binding to FcRs. Cells were further stained with the following fluorochrome-conjugated antibodies: CD45 (30-F11), CD3e (145-2C11), CD11b (M1/70), F4/80 (BM8), CD86 (GL1), CD206 (C068C2), MHC-II (AF6-120) and Yellow Fluorescence (all from eBioscience, San Diego, California, United States of America). A flow cytometer sold under the tradename LSRFORTESSA™ (BD Biosciences, San Jose, California, United States of America) was used for cell acquisition and data analysis was carried out with FlowJo software (Tree Star, Ashland, Oregon, United States of America).

Immunofluorescence assays: Tumor sections were air-dried for at least 1 h and then fixed in acetone for 10 min at 20° C. After being blocked with 20% donkey serum, the sections were incubated with individual primary antibodies against CD47 (MIAP301, Biolegend, San Diego, California, United States of America) or F4/80 (BM8), CD86 (GL1), CD206 (C068C2), all from eBiosciences (San Diego, California, United States of America) overnight at 4° C., followed by incubation with dye-conjugated secondary antibodies for 1 h at r.t. After being stained with DAPI for another 10 min, the sections were then washed twice with PBS and observed under CLSM.

Anti-tumor efficacy: For the evaluation of Hf-DBP-modulated macrophage therapy, 2×10⁶ CT26 cells were injected into the right flank subcutaneous tissues of Balb/c mice on day 0 as a single tumor CT26 model. When the tumors reached 100-150 mm³ in volume, Hf-DBP, IMD@Hf-DBP, Hf-DBP/αCD47, or IMD@Hf-DBP/αCD47 at an equivalent Hf dose of 0.1 μmol or equivalent amount of IMD or αCD47 was injected intratumorally. Mice without any treatment served as a control. 12 hours after injection, mice were anaesthetized with 2.5% (v/v) isoflurane and the tumors were irradiated with X-rays at a dose of 2 Gy for 2 consecutive days. The tumor sizes were measured with a caliper every day and the tumor volumes were calculated as (width²×length)/2. Body weight of each group was monitored every day. Mice were sacrificed on Day 25 and the excised tumors were photographed and weighed. Tumors were sectioned for hematoxylin-eosin staining (H&E) and immunofluorescence analysis.

### Example 7

### Combination of IMD@Hf-DBP and IMD@Hf-DBP/α-CD47 with Checkpoint Blockade Immunotherapy

Abscopal effect: For the evaluation of nMOF-mediated macrophage therapy combined with checkpoint blockade immunotherapy, a bilateral syngeneic CT26 model was established by injecting 2×10⁶ and 1×10⁶ cells into the right and left flank subcutaneous tissues of Balb/c mice on day 0 to mimic primary and distant tumors, respectively. When the primary tumors reached 100-150 mm³ in volume, IMD/αCD47, Hf-DBP/αCD47, IMD@Hf-DBP, or IMD@Hf-DBP/αCD47 at an equivalent dose of 0.2 μmol or PBS was injected intratumorally. 12 hours after injection, mice were anaesthetized with 2% (v/v) isoflurane and the primary tumors were irradiated once with X-rays at a dose of 2 Gy for 2 consecutive days of irradiation. Anti-PD-L1 antibody was given every three days by intraperitoneal injection at a dose of 75 μg/mouse for 2 injections. Mice without treatment served as controls. Mice were euthanized on Day 25. Tumors and major organs were harvested for immune analysis.

ELISPpot assay: Tumor-specific immune responses to IFN-γ were measured in vitro by ELISpot assay (Mouse IFN-γ ELISPOT READY-SET-GO!™; Cat. No. 88-7384-88; eBioscience, San Diego, California, United States of America). A Millipore Multiscreen HTS-IP plate (Millipore Sigma, Burlington, Massachusetts, United States of America) was coated overnight at 4° C. with anti-Mouse IFN-γ capture antibody. Single-cell suspensions of splenocytes were obtained from CT26 tumor-bearing mice treated with PBS(−), PBS(+), αPD-L1(+), IMD@Hf-DBP/αCD47(+), IMD@Hf-DBP/αCD47(−)+αPD-L1, IMD@Hf-DBP/αCD47(−)+αPD-L1, and seeded onto the antibody-coated plate at 2×10⁵ cells per well. Cells were incubated with or without SPSYVYHQF (SEQ ID NO: 4) stimulation (10 mg/ml; in purity >95%; PEPTIDE 2.0, Chantilly, Virginia, United States of America) at 37° C. for 42 h and then the suspension was discarded. The plate was then incubated with biotin-conjugated anti-IFN-γ detection antibody at r.t. for 2 h, followed by incubation with Avidin-HRP at r.t. for 2 h. 3-amino-9-ethylcarbazole substrate solution (Sigma, St. Louis, Missouri, United States of America, Cat. AEC101) was added for cytokine spot detection.

Profiling of adaptive immunity: Tumors were harvested, treated with 1 mg/ml collagenase I (Gibco Laboratories, Gaithersburg, Maryland, United States of America) for 1 h in a 37° C. water bath, and ground by the rubber end of a syringe. Cells were filtered through 70 m nylon mesh filters and washed with PBS. The single-cell suspension was incubated with anti-CD16/32 (clone 93; eBiosciences, San Diego, California, United States of America) to reduce nonspecific binding to FcRs. Cells were further stained with the following fluorochrome-conjugated antibodies: CD45 (30-F11), CD3e (145-2C11), CD4 (GK1.5), CD8 (53-6.7), B220 (RA3-6B2), Nkp46 (29A1.4), and Yellow Fluorescence staining solution (all from eBioscience, San Diego, California, United States of America). A flow cytometer sold under the tradename LSRFORTESSA™ (BD Biosciences, San Jose, California, United States of America) was used for cell acquisition and data analysis was carried out with FlowJo software (Tree Star, Ashland, Oregon, United States of America).

### Example 8

### Discussion of Examples 1-7

As the immune systems primary defense, macrophage phagocytose nascent cancerous cells to maintain host homeostasis (Alavena et al. 2008). However, macrophages' ability to target cancer cells is limited by overexpression of the “don't-eat-me” CD47 checkpoint signalling molecule on tumor cell surfaces to escape immune surveillance (Jaiswal et al. 2009) and promote an immunosuppressive tumor microenvironment (TME) with the preponderance of anti-inflammatory (tumor promoting) M2 macrophages (Chao et al. 2010). Immunostimulatory therapies are explored to reshape the TME and reverse immunosuppression with the goal of activating anti-tumor immune responses (Lou et al. 2019; Chen et al. 2015; Louttit et al. 2019; and Nam et al. 2019).

One such treatment is IMB, a hydrophobic small molecule drug capable of repolarizing innate immunity by activating the toll-like receptor-7 (TLR-7) pathway (Rodell et al. 2018; and O'Neill et al. 2013). TLR-7 agonists such as IMD can repolarize M2 macrophages to pro-inflammatory (anti-tumor) M1 macrophages, which facilitates phagocytosis, inflammation, and antigen presentation. In addition, macrophage checkpoint inhibition is proposed as an anti-tumor treatment by promoting phagocytosis of dying tumor cells and release tumor antigens (Chen et al. 2019a; and Liu et al., 2015). Indeed, blockade of the CD-47 signaling pathway with an anti-CD47 antibody (αCD47) is under clinical investigations (Chen et al. 2019b; Kojima et al. 2016; and Feng et al. 2019). However, systemically toxic IMD and αCD47 therapies have been limited by inadequate anti-tumor efficacy and undesired side effects even via intratumoral injection (Liu et al., 2015; Kamath et al. 2018; and Xiong et al. 2011).

The presently disclosed subject matter describes the use of a nMOF for the co-delivery of IMD and αCD47 to the tumor cells to augment the radiotherapy-radiodynamic therapy (RT-RDT) effect from the nMOF and low-dose X-ray irradiation. In particular, nMOFs with heavy-metal secondary build units (SBUs) and photosensitizing ligands can mediate RT-RDT by enhancing X-ray energy deposition and generating multiple reactive oxygen species (ROS).

IMD@Hf-DBP/αCD47 was synthesized by sequential Hf-DBP nMOF surface modification, IMD loading, and αCD47 adsorption. IMD@Hf-DBP/αCD47 plus X-ray irradiation elicited strong RT-RDT effect to cause ICD of tumor cells and enhance the immune-modulation effects of the TLR-7 agonist and the CD-47 blocker. See FIG. 1. Further combination with anti-PD-L1 immune checkpoint blockade (CBI) completely eradicated both primary and distant tumors on a bilateral colorectal tumor model.

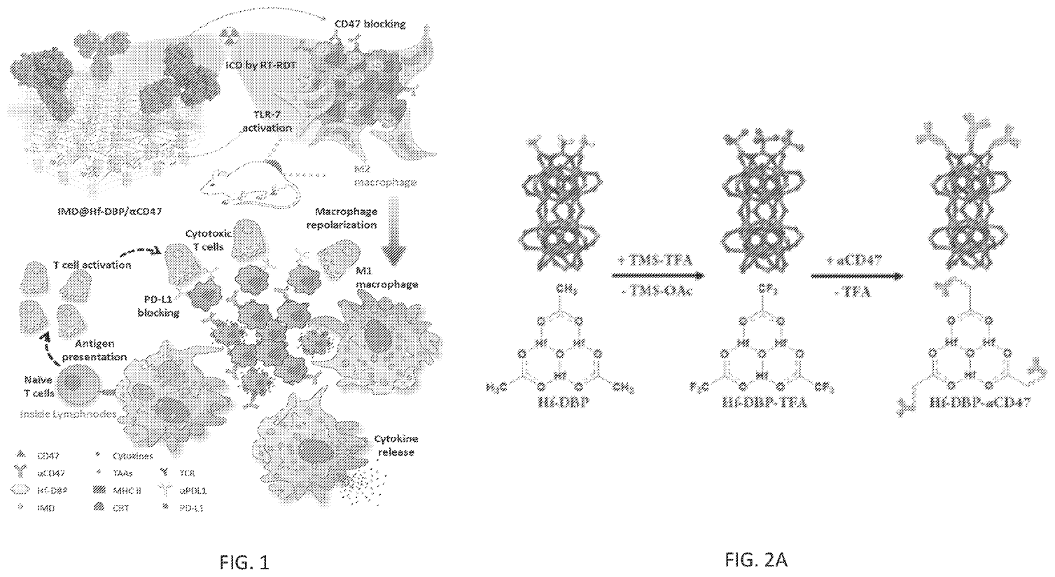

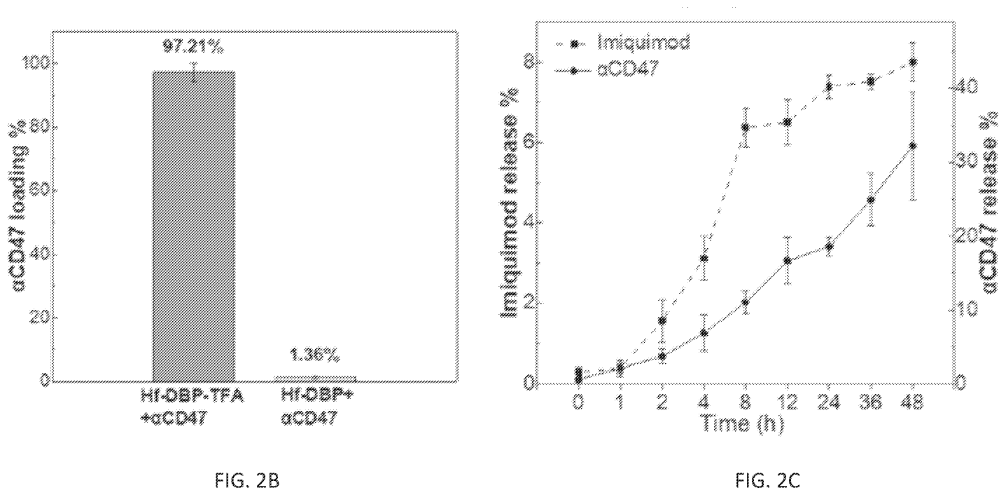

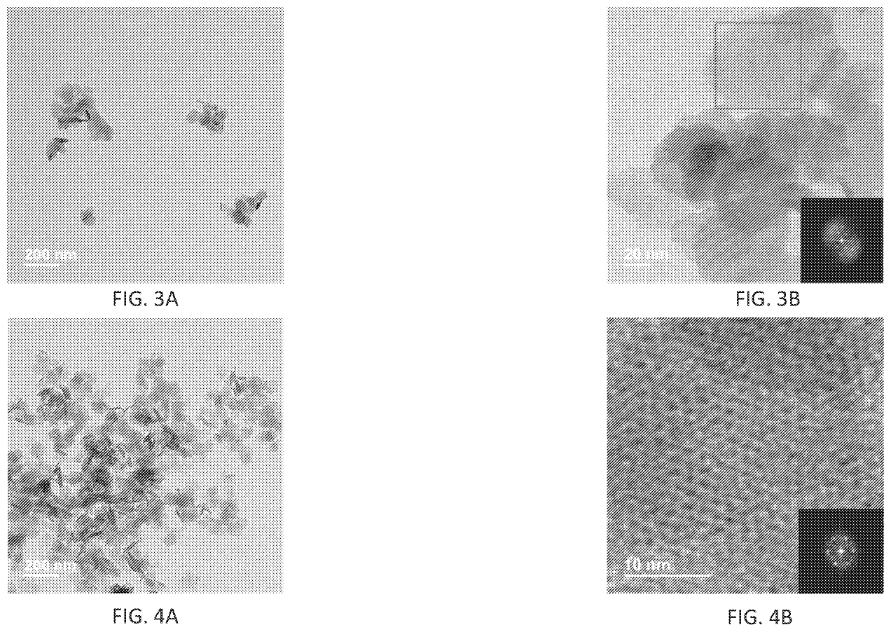

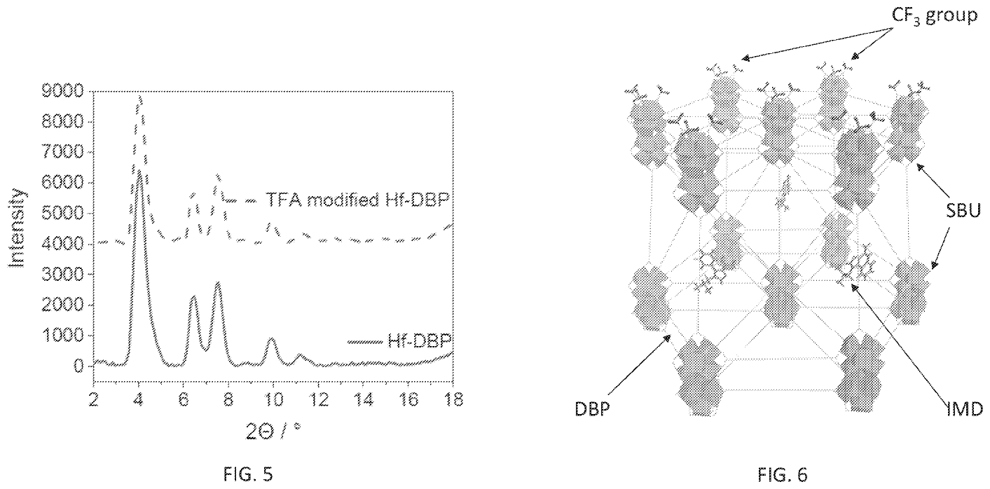

Hf-DBP nMOF with acetate (OAc) capping groups was synthesized as previously reported (Lu et al., 2014) and exhibited an empirical formula of Hf₁₂(μ₃-O)₈(μ₃-OH)₈(μ₂-OH)₆(AcO)_{3.5}(DBP)_{6.8}(OH)_{0.9}(OH₂)_{0.9}. Nanoplates of Hf-DBP were formed by connecting Hf₁₂ SBUs with DBP ligands in an hcp-like stacking pattern. The Hf₁₂ SBUs on the surface were terminated by OAc groups (see FIG. 2A) with a ζ-potential of −23.3±1.0 mV in H₂O, which prevented the loading of αCD47 on the surface of Hf-DBP nanoplates. See FIG. 2B. Hf-DBP was treated with trimethylsilyl trifluoroacetate (TMS-TFA) to afford TFA-modified Hf-DBP by replacing >90% of the OAc groups with TFA groups as determined by ¹H and ¹⁹F NMR spectroscopy. TEM imaging and PXRD studies showed that the morphology and crystallinity of Hf-DBP were maintained after surface modification. See FIGS. 3A, 3B, 4A, 4B, and 5. The weekly coordinating TFA groups can be replaced by carboxylate groups in proteins or phosphate groups on nucleic acids. Hf-DBP-TFA showed a nearly complete adsorption of αCD47 (97.2%) by BCA assays. See FIG. 2B.

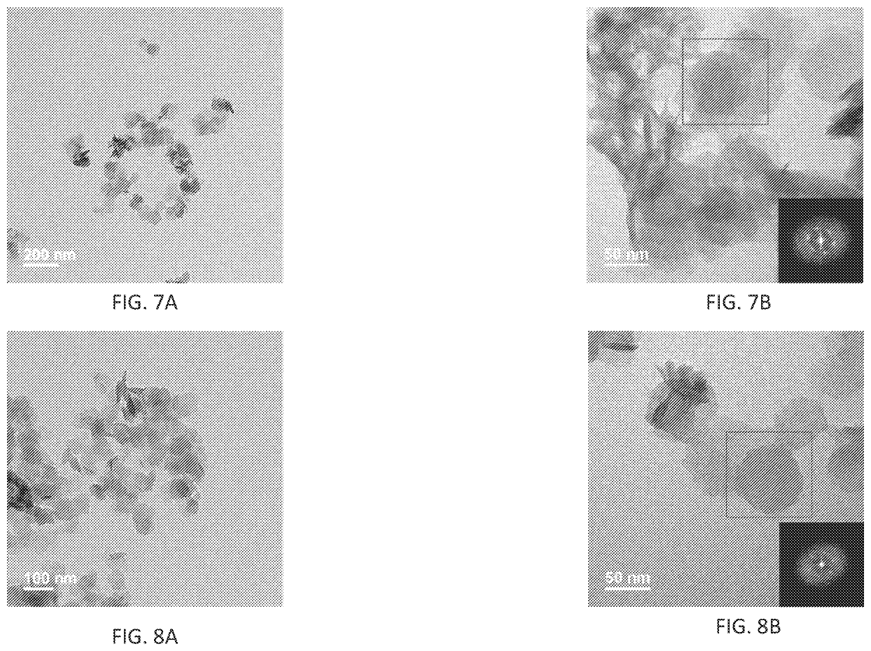

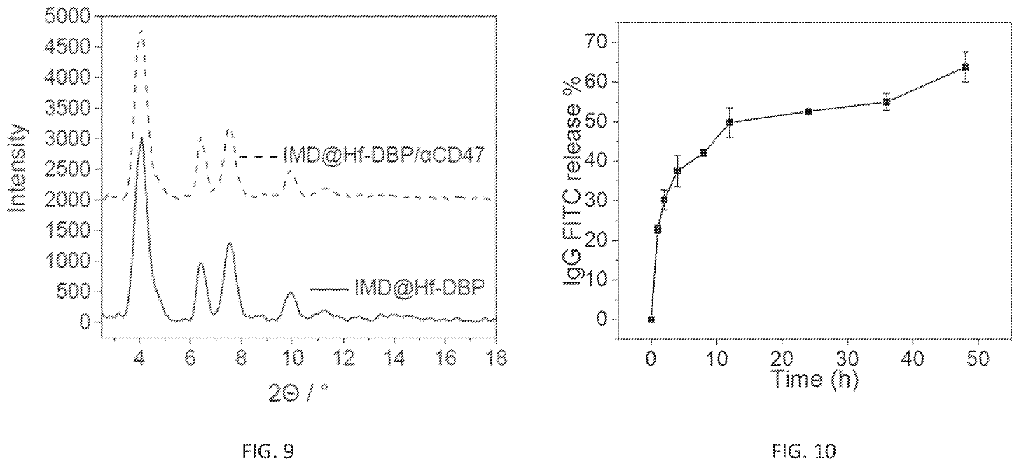

IMD was loaded into the pores of TFA-modified Hf-DBP through sonication in ethanol to afford IMD@Hf-DBP (see FIG. 6) with ˜9 wt % IMD loading as determined by ¹H NMR and UV-vis spectroscopy and thermogravimetric analysis. The addition of αCD47 to a PBS suspension of IMD@Hf-DBP with vortexing afforded IMD@Hf-DBP/αCD47 with 7.5 wt % αCD47 loading. IMD@Hf-DBP and IMD@Hf-DBP/αCD47 maintained the morphology and crystallinity of Hf-DBP based on TEM images and PXRD patterns. See FIGS. 7A, 7B, 8A, 8B, and 9. See also Table 1, above.

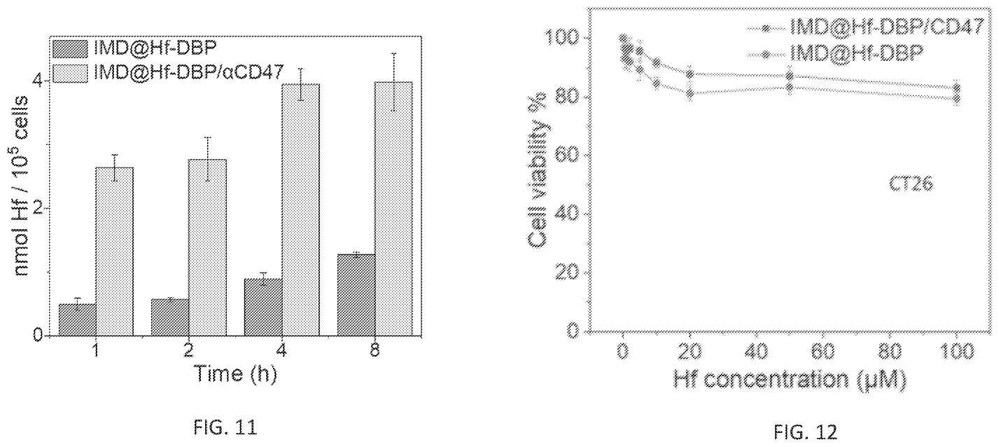

The release profiles of IMD and αCD47 of IMD@Hf-DBP/αCD47 in PBS at 37° C. were determined by liquid chromatography-mass spectrometry (LC-MS) and bicinchoninic acid (BCA) assays, respectively. See FIG. 2C. Approximately 8% IMD was slowly released in 48 hours, which is ideal for maintaining a high local IMD concentration to continuously repolarize M2 to M1 macrophages. Approximately 30% and 60% αCD47 was released in 48 hours in PBS or PBS with 10% (v/v) FBS, respectively, likely via substitution by phosphate coordination to Hf₁₂ SBUs. See FIG. 10. IMD@Hf-DBP/αCD47 showed high cellular uptake. See FIG. 11.

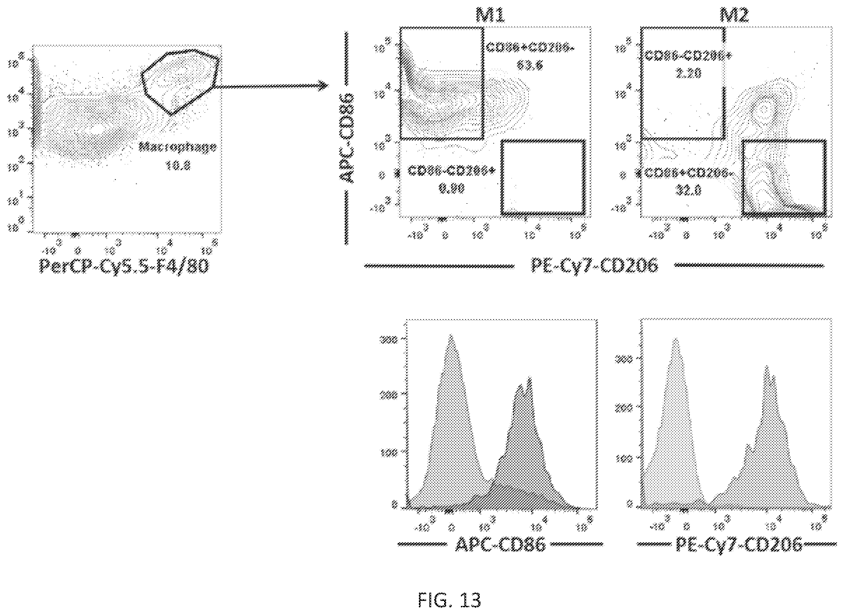

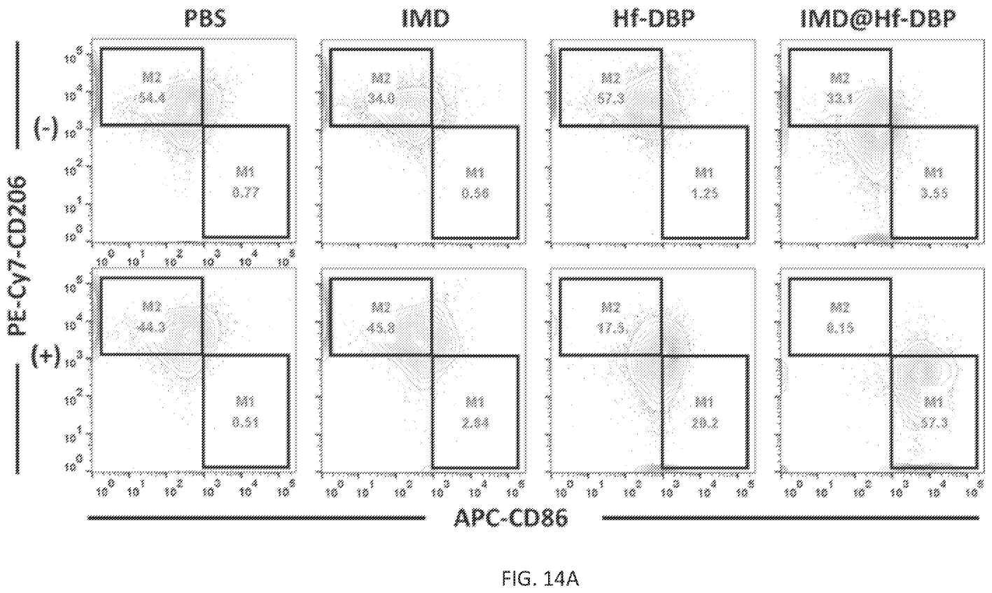

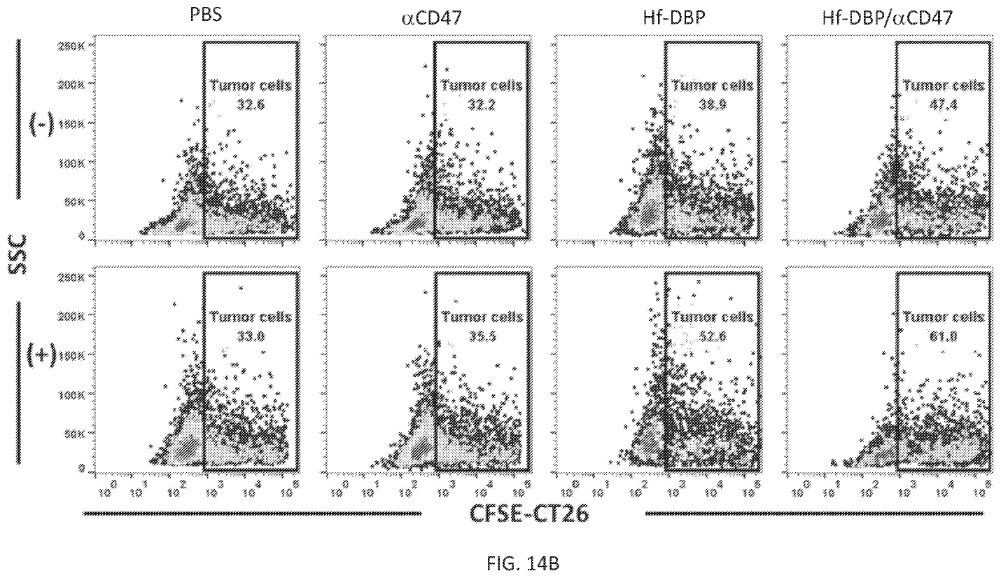

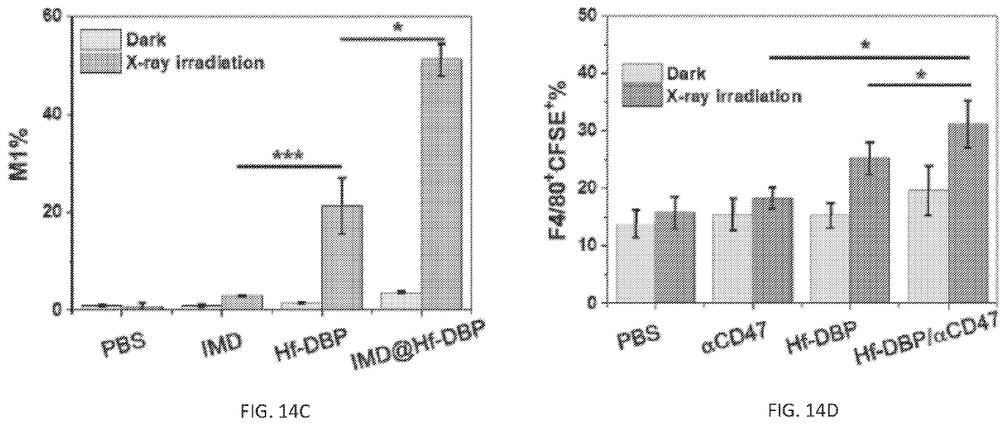

Studies were performed to determine if released IMD and αCD47 could target macrophages and tumor cells, respectively, for anti-tumor effects in vitro. First, bone-marrow-derived monocytes were harvested to differentiate them into either M1 or M2 macrophages. Gated from F4/80⁺ cells, M1 or M2 cells presented CD86⁺CD206⁻ or CD86⁻CD206⁺ phenotypes, respectively. See FIG. 13. Murine colorectal cancer CT26 cells were treated with IMD@Hf-DBP, Hf-DBP, or IMD and then irradiated with 2 Gy of X-ray (hereafter + denotes X-ray irradiation). M2 macrophages were then co-cultured with the treated CT26 cells for 24 h, and immunostained for flow cytometric analysis. As shown in FIG. 14A, CT26 cells treated with IMD@Hf-DBP(+) induced stronger macrophage repolarization with a higher M1/M2 ratio of 382 over other groups (0.01 to 1.15). Interestingly, Hf-DBP(+) induced a higher M1 population than IMD(+) (see FIG. 14C), reflecting the intrinsic property of RT-RDT to repolarize macrophages from M2 to M1 phenotype.

CT26 cells were next treated with Hf-DBP/αCD47, Hf-DBP, or αCD47 to evaluate phagocytosis. The translocation of calreticulin to cell surface as the “eat-me” signal was confirmed in Hf-DBP(+) and Hf-DBP/αCD47(+) groups. M1 macrophages were co-cultured with the treated CT26 cells and significantly increased phagocytosis of CFSE-labeled CT26 cells was observed by PerCP-Cy5.5-conjugated F4/80-labeled M1 macrophages in Hf-DBP/αCD47(+) group, indicating enhanced phagocytosis promoted by the exposure of “eat me” signal (calreticulin) and the blocking of “don't eat me” signal (CD47) on tumor cell surfaces. See FIGS. 14B and 14D. Similar results were observed under CLSM, in which Hf-DBP-αCD47(+) group showed most CT26 cells phagocytosed by PerCP-Cy5.5-labeled M1 macrophages.

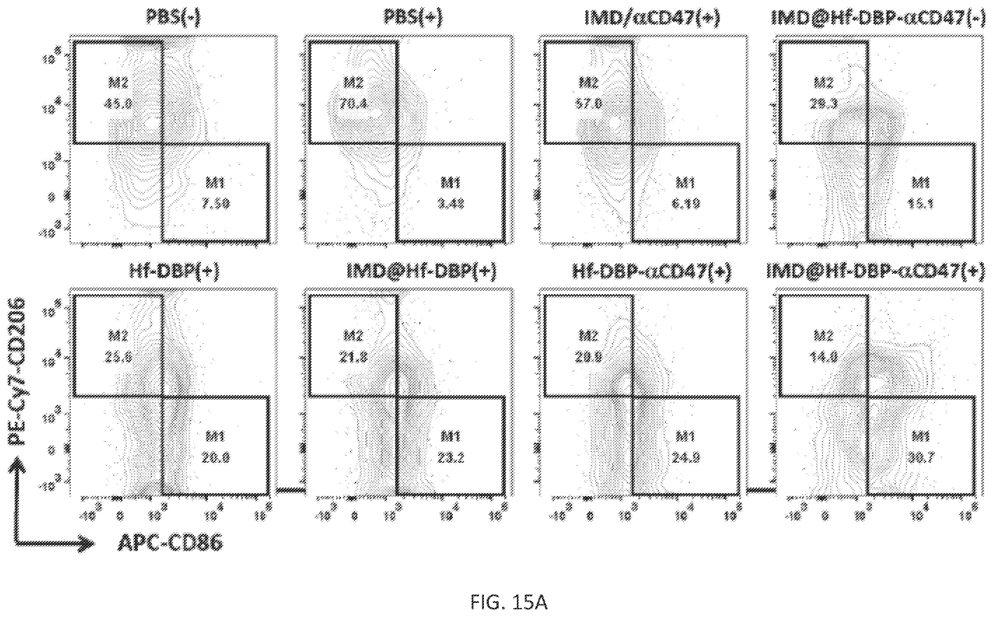

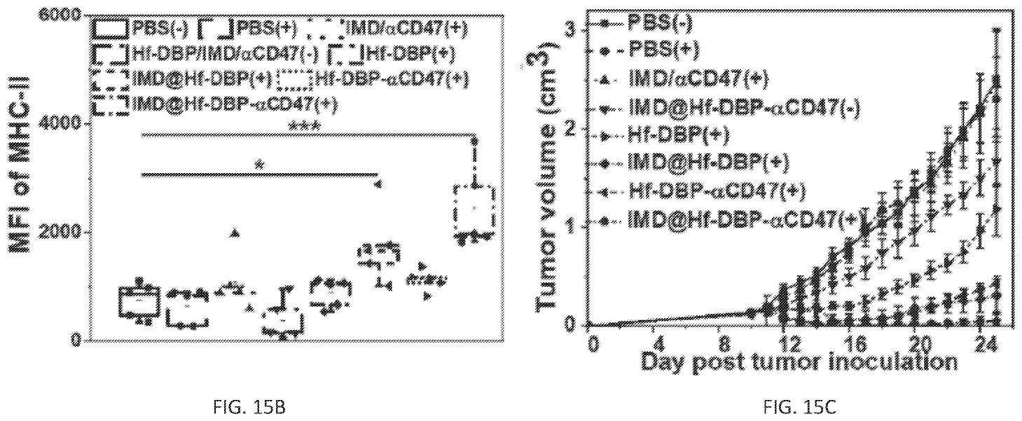

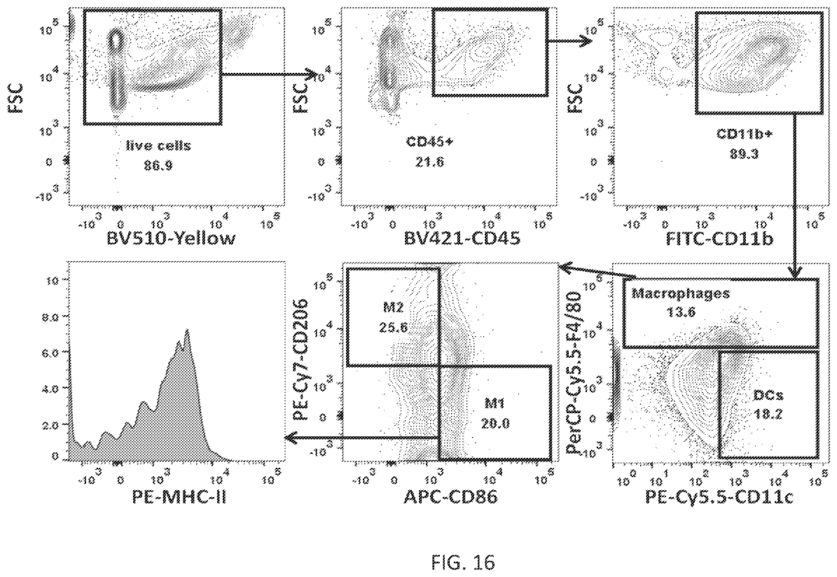

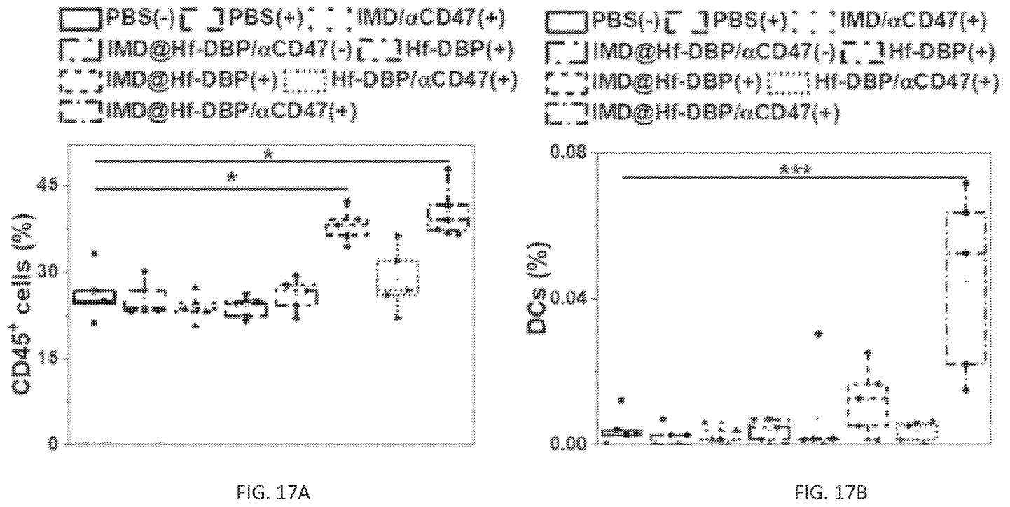

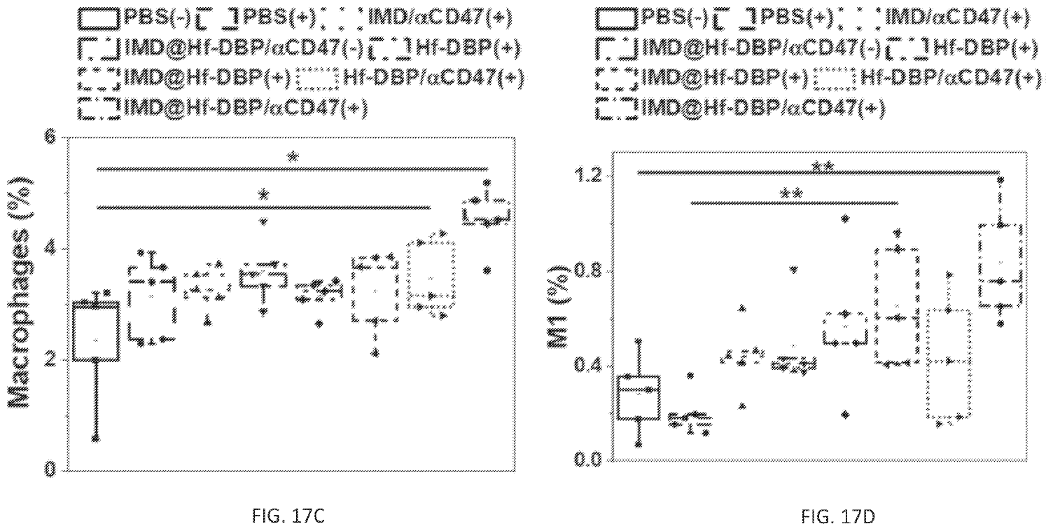

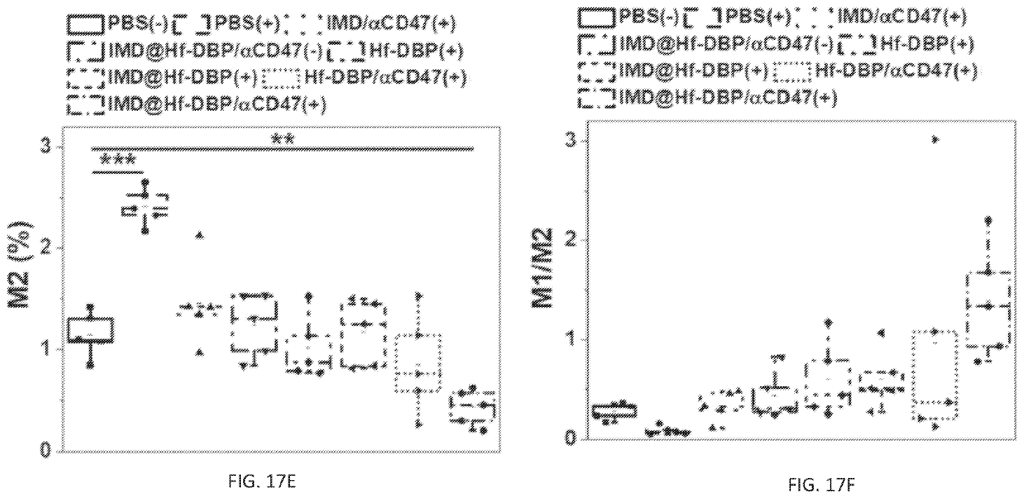

IMD@Hf-DBP/αCD47 plus X-ray was tested to see if it could reshape TME by modulating macrophage for anti-tumor immune activation. PBS, IMD/αCD47, Hf-DBP, Hf-DBP/αCD47, or IMD@Hf-DBP/αCD47 was intratumorally injected into CT26-tumor bearing BALB/c followed by 2 Gy/fraction X-ray irradiation for 2 consecutive days. Tumor section slides were immunostained to detect CD47 blockade 48 h post the first irradiation. While IMD/αCD47(+) slightly lowered the fluorescence signal of CD47 compared to PBS controls, Hf-DBP/αCD47(+) and IMD@Hf-DBP/αCD47(+) strongly blocked CD47, indicating higher blocking efficacy of Hf-DBP-delivered αCD47. Interestingly, Hf-DBP(+) treatment also decreased CD47. Tumor-infiltrating innate immune cells were profiled 48 hours post the first irradiation. See FIG. 16. IMD@Hf-DBP/αCD47(+) treatment statistically increased tumor-infiltrating leukocytes, dendritic cells, and macrophages (see FIGS. 15A and 17A-17C), indicating an inflammatory TME with enhanced infiltration of antigen presenting cells. Furthermore, IMD@Hf-DBP/αCD47(+) treatment significantly increased M1 macrophage and decreased M2 macrophage, indicating in vivo macrophage repolarization by synergistic nMOF-mediated RT-RDT, TLR-7 agonist, and CD-47 blockade. See FIG. 15A and FIGS. 17D-7F. Interestingly, a significant increase of M2 phenotype was observed in PBS(+) group, suggesting a more immunosuppressive TME induced by low-dose X-ray alone. See FIG. 17E. Additionally, significant increases of total macrophages (see FIG. 17C) and M1 phenotype (see FIG. 17D) were found in Hf-DBP/αCD47(+) or IMD@Hf-DBP(+) groups, respectively, indicating nMOF delivery enhances macrophage modulation effects of IMD and αCD47. Macrophage repolarization was confirmed by CLSM imaging of immunostained tumor section slides. Moreover, M1 macrophages showed significantly increased MHC-II expression in both IMD@Hf-DBP(+) and IMD@Hf-DBP/αCD47(+) groups (see FIG. 15B), suggesting their enhanced function in antigen presentation.

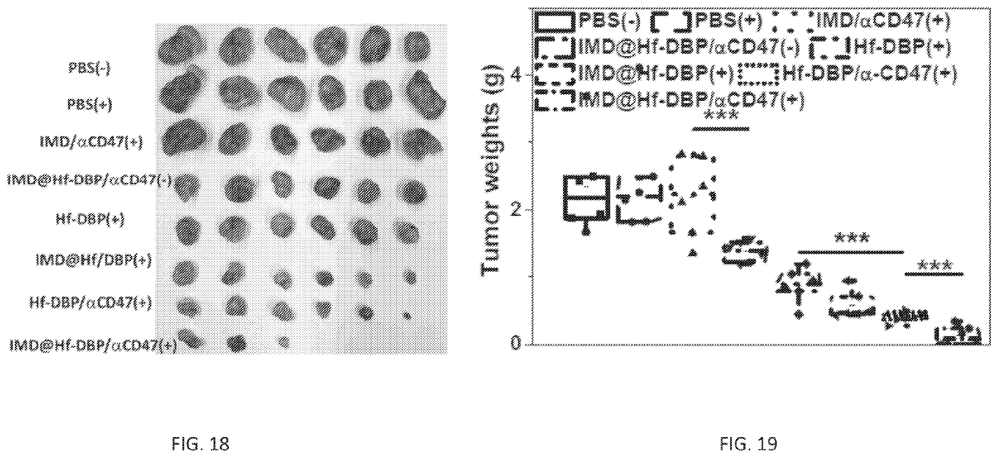

The anti-tumor efficacy of IMD@Hf-DBP/αCD47(+) was evaluated on a CT26 tumor model. When the tumors reached ˜150 mm³, IMD/αCD47, IMD@Hf-DBP, Hf-DBP/αCD47, or IMD@Hf-DBP/αCD47 was intratumorally injected and then irradiated with 2 Gy/fraction X-ray for 2 consecutive days. These treatments showed no systemic toxicity, as evidenced from steady body weights. IMD@Hf-DBP(+) and Hf-DBP/αCD47(+) showed strong tumor suppression with tumor growth inhibition (TGI) indices of 83.3% and 89.3%, respectively, over PBS(−) control on Day 25. IMD@Hf-DBP/αCD47(+) treatment effectively eradicated tumors with a TGI of 98.2% and a 50% cure rate. See FIG. 15C. See also Table 2, below. Hf-DBP(+) and IMD@Hf-DBP/αCD47(−) showed moderate efficacy with TGI values of 52.8% and 33.8%, respectively, but IMD/αCD47(+) showed minimal effect with a TGI of 2.1%. These results support the enhanced antitumor efficacy of nMOF-delivered IMD and αCD47. The averaged weights of excised tumors on Day 25 were 2.34±0.73, 2.44±0.86, 2.18±0.60, 1.38±0.15, 0.87±0.26, 0.58±0.21, 0.41±0.08, and 0.12±0.15 g for PBS(−), PBS(+), IMD/αCD47(+), IMD@Hf-DBP/αCD47(−), Hf-DBP(+), IMD@Hf-DBP(+), Hf-DBP/αCD47(+), and IMD@Hf-DBP/αCD47(+) groups, respectively. See FIGS. 18 and 19. H&E staining indicated severe necrosis of tumor slices from IMD@Hf-DBP/αCD47(+) treatment. IMD@Hf-DBP/αCD47(+) treatment thus repolarizes macrophages to reshape intratumoral immunity to afford superb anti-tumor efficacy.

TABLE 2
Student T-test analysis and p-values of tumor volumes of
single CT26-bearing mice after treatments at end point.

| Comparison | p-value |
|---|---|
| PBS(−) vs. PBS(+) | 0.264 |
| PBS(+) vs. IMD/αCD47(+) | 0.222 |
| IMD@Hf-DBP/αCD47(−) vs. IMD/αCD47(+) | 0.00185 |
| IMD@Hf-DBP/αCD47(−) vs. Hf-DBP(+) | 0.00109 |
| IMD@Hf-DBP(+) vs. Hf-DBP/αCD47(+) | 0.182 |
| Hf-DBP(+) vs. IMD@Hf-DBP(+) | 0.00084 |
| Hf-DBP(+) vs. Hf-DBP/αCD47(+) | 0.00012 |
| IMD@Hf-DBP/αCD47(+) vs. Hf-DBP(+) | <0.0001 |
| IMD@Hf-DBP/αCD47(+) vs. IMD@Hf-DBP/CD47(−) | <0.0001 |
| IMD@Hf-DBP/αCD47(+) vs. IMD@Hf-DBP(+) | 0.0359 |
| IMD@Hf-DBP/αCD47(+) vs. Hf-DBP/αCD47(+) | 0.00079 |

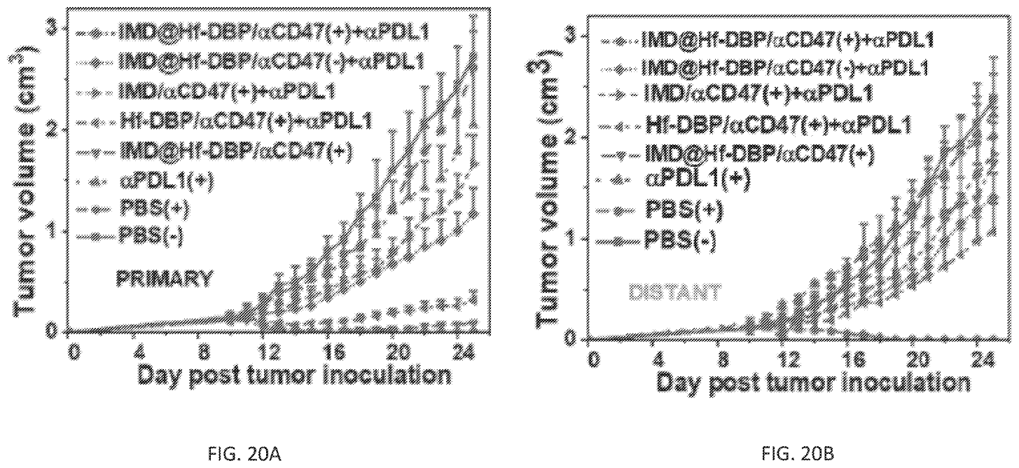

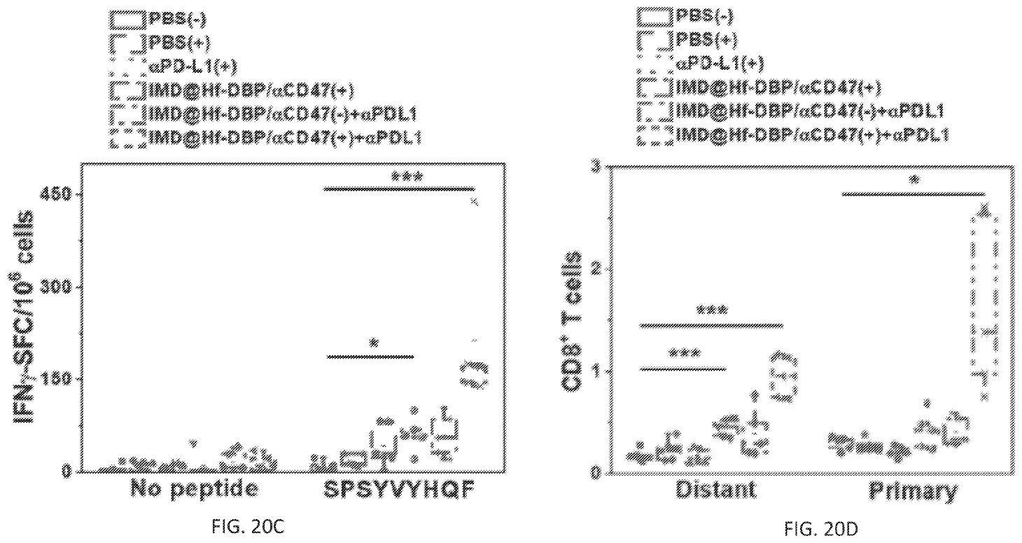

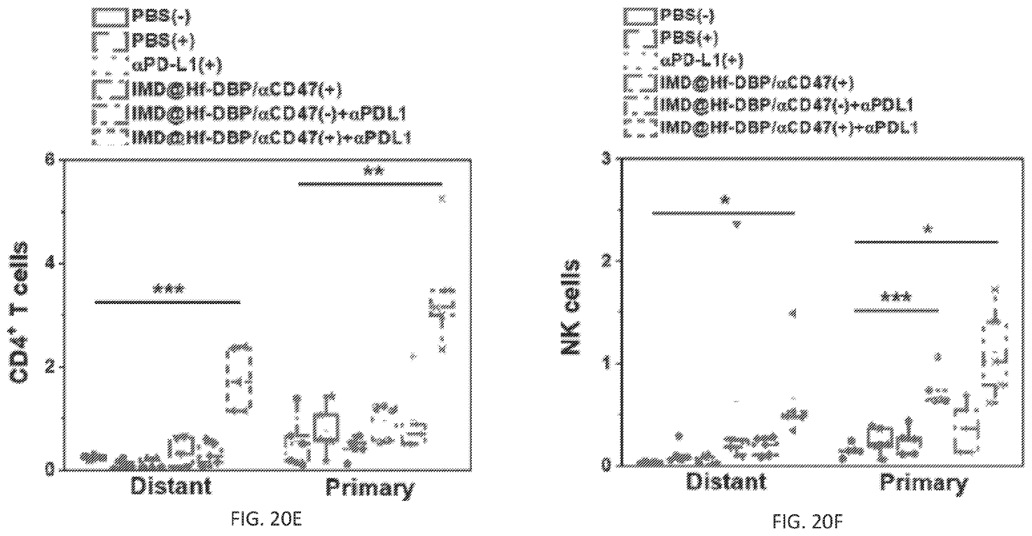

Synergy between the macrophage therapy and CBI was investigated. IMD@Hf-DBP/αCD47(+) or each of the controls was injected into the primary tumors of the bilateral CT26 tumor model followed by two intraperitoneal injections of αPDL1 and two fractions of X-ray at 2 Gy/fraction. As shown in FIG. 20A, αPDL1(+) treatment did not exhibit significant difference from PBS(+/−) controls. IMD@Hf-DBP/αCD47(+) treatment effectively suppressed primary tumors but had no effect on distant tumors. In contrast, IMD@Hf-DBP/αCD47(+)+αPDL1 treatment completely eradicated both primary and distant tumors. IMD@Hf-DBP/αCD47(+) or IMD@Hf-DBP/αCD47(−)+αPDL1 only moderately controlled tumor growth. See FIG. 20B. See also Table 3, below. Synergistic actions of Hf-DBP-mediated RT-RDT, reversal of macrophage-related immunosuppression by IMD, and blockade of macrophage checkpoint by αCD47 thus form an immunological hot bed for effective CBI to lead to superb abscopal effect.

TABLE 3
Student T-test analysis and p-values of tumor volumes of bilateral CT26-
bearing mice after treatments at end point (* = <0.0001).

| Comparison | primary | distant |
|---|---|---|
| PBS(−) vs. PBS(+) | 0.227 | 0.346 |
| PBS(+) vs. αPDL1(+) | 0.153 | 0.600 |
| PBS(+) vs. IMD@Hf-DBP/αCD47(+) | <0.0001 | 0.526 |
| IMD/αCD47(+) + αPDL1 vs. αPDL1(+) | 0.073 | 0.290 |
| IMD@Hf-DBP/αCD47(+) + αPDL1 vs. PBS(+) | * | * |
| IMD@Hf-DBP/αCD47(+) + αPDL1 vs. αPDL1(+) | * | * |
| IMD@Hf-DBP/αCD47(+) + αPDL1 vs. IMD@Hf- | * | * |
| DBP/αCD47(+) | | |
| IMD@Hf-DBP/αCD47(+) + αPDL1 vs. Hf- | * | * |
| DBP/αCD47(+) + αPDL1 | | |
| IMD@Hf-DBP/αCD47(+) + αPDL1 vs. | * | * |
| IMD/αCD47(+) + αPDL1 | | |
| IMD@Hf-DBP/αCD47(+) + αPDL1 vs. IMD@Hf- | * | * |
| DBP/αCD47(−) + αPDL1 | | |

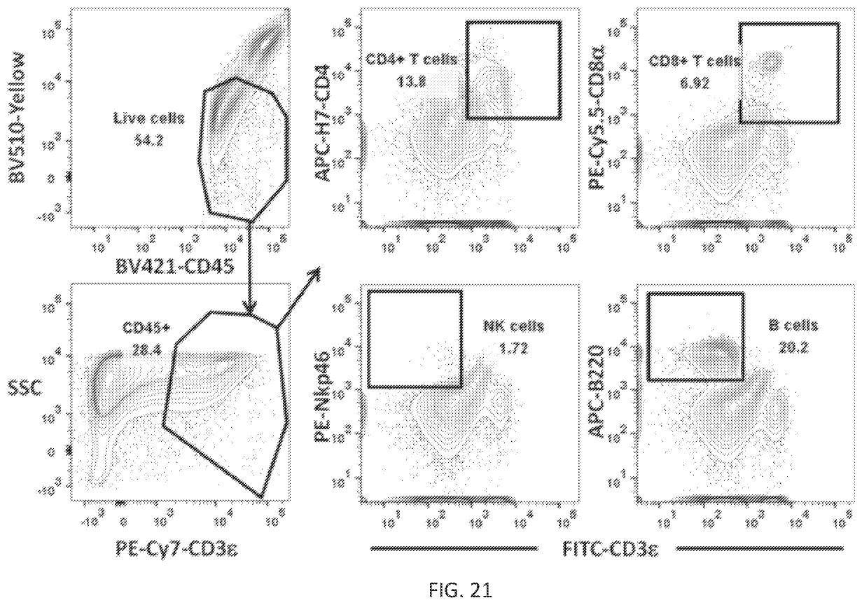

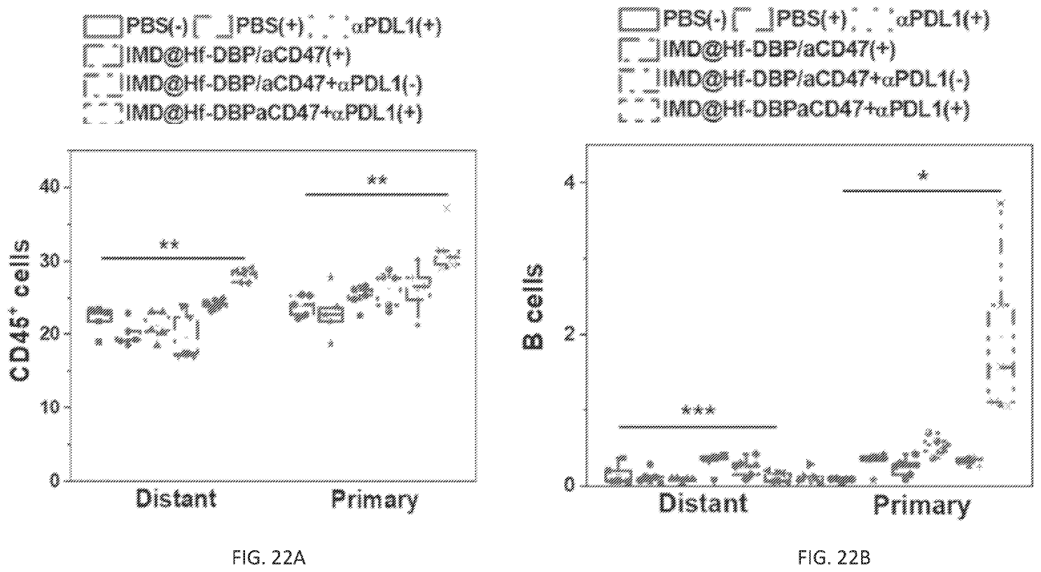

The anti-tumor immunity induced by IMD@Hf-DBP/αCD47(+)+αPDL1 was probed. First, an ELISpot assay was performed to determine specific anti-tumor immunity by detecting IFN-γ producing cytotoxic T cells in splenocytes 12 days after the first irradiation. Stimulated with cytotoxic T cell epitope AH1 (SPSYVYHQF) (SEQ ID NO: 4) expressed by CT26 tumor cells, cytotoxic T cell formed IFN-γ spots for counting. Significant increases of antigen-specific IFN-γ producing T cells were observed in IMD@Hf-DBP/αCD47(+) (61.6±28.6) and IMD@Hf-DBP/αCD47(+)+αPD-L1 (213.8±126.9/10⁶ cells) groups compared to 8.0±9.3/10⁶ cells for PBS(−), suggesting induction of strong tumor-specific adaptive immunity. See FIG. 20C. Flow cytometric analyses (see FIG. 21) showed significant increase of tumor-infiltrating leukocytes (see FIG. 22A), CD8⁺ T cells (see FIG. 20D), and CD4⁺ T cells (see FIG. 20E) in both primary and distant tumors of IMD@Hf-DBP/αCD47(+)+αPD-L1 group. Interestingly, natural killer (NK, see FIG. 20F) and B cells (see FIG. 22B) also significantly increased in both primary and distant tumors. These results confirm that IMD@Hf-DBP/αCD47(+)-mediated local macrophage therapy and systemic CBI augment the infiltration of effector T cells, B cells, and NK cells to activate anti-tumor immunity and generate a strong abscopal effect.

In summary, the presently disclosed subject matter provides a new strategy to modify nMOF surfaces for biomacromolecule delivery. Loading of IMD in the pores and αCD47 on the surface of modified Hf-DBP led to superb anti-tumor efficacy by immunomodulation of macrophages and activation of innate immunity. Hf-DBP-mediated RT-RDT and the slowly released IMD repolarized immunosuppressive M2 macrophages to immunostimulatory M1 macrophages, while the released αCD47 blocked the “don't eat me” signal on tumor cells for improved phagocytosis. When combined with αPD-L1, IMD@Hf-DBP/αCD47(+)-mediated macrophage therapy was extended to systemically eradicate tumors on a bilateral CT26 tumor model. Accordingly, it appears that the “three-in-one” treatment for macrophage modulation can serve as a strategy to tune the TME for synergistic combination with immunotherapies.

### Example 9

### Materials and Methods for Examples 10-16

Cell lines and animals: Murine colorectal adenocarcinoma cell line MC38, Lewis lung carcinoma cell line LL2, and melanoma cell line B16F10 were purchased from the American Type Culture Collection (Rockville, Maryland, United States of America). Murine pancreatic cancer cell line Panc02 was kindly provided by Dr. Hans Schreiber from Department of Pathology, University of Chicago (Chicago, Illinois, United States of America). MC38-ova cell line [OVA(257-264)-ZSGREEN] was generated by transfection of MC38 cells with LZRS-based retrovirus. All of the cells were cultured in Dulbecco's Modified Eagle's Medium (DMEM) medium (GE Healthcare, Chicago, Illinois, United States of America) supplemented with 10% FBS, 100 U/mL penicillin G sodium and 100 μg/mL streptomycin sulfate. Cells were cultured in a humidified atmosphere containing 5% C02 at 37° C. Mycoplasma was tested before use by a kit sold under the tradename MYCOALERT™ (Lonza Walkersville, Inc., Walkersville, Maryland, United States of America) C57BL/6 mice (6-8 weeks) were obtained from Harlan-Envigo Laboratories, Inc (Indianapolis, Indiana, United States of America).

Synthesis of H₂DBB-Ir-F and H₂DBB-Ir Ir(DBB)[dF(CF₃)ppy]₂⁺ [H₂DBB^{F}_Ir, DBB=4,4′-di(4-benzoato)-2,2′-bipyridine; dF(CF₃)ppy=2-(2,4-difluorophenyl)-5-(trifluoromethyl)pyridine] was synthesized as shown in Scheme 1, below according to the literature report (Zhu et al., 2018). ¹H NMR (500 MHz, DMSO-d₆): δ 9.08 (d, 2H), 8.76 (d, 2H), 8.49 (d, 2H), 8.44 (d, 2H), 8.15 (s, 2H), 8.02 (d, 4H), 7.82 (s, 2H), 7.62 (d, 4H), 7.12 (t, 2H), 5.91 (d, 2H). Ir(H₂DBB)(ppy)₂⁺ (H₂DBB-Ir, DBB=4,4′-di(4-benzoato)-2,2′-bipyridine; ppy=2-phenylpyridine) was synthesized as shown in Scheme 1, below, according to the literature report (Lan et al., 2019b). ¹H NMR (500 MHz, DMSO-d₆): δ 9.05 (d, 2H), 8.67 (d, 2H), 8.28 (d, 2H), 8.07 (s, 2H), 7.96 (m, 8H), 7.89 (d, 2H), 7.52 (d, 4H), 7.17 (t, 2H), 7.09 (t, 2H), 6.98 (t, 2H), 6.33 (d, 2H

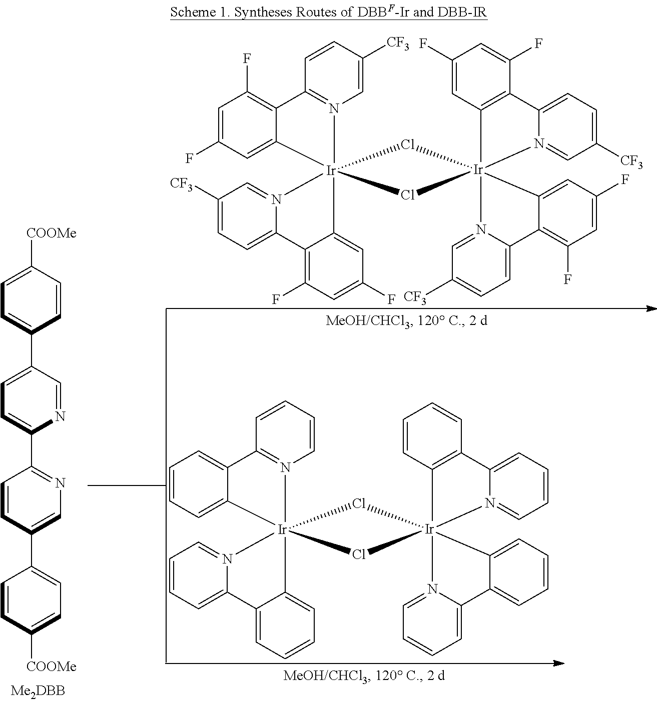

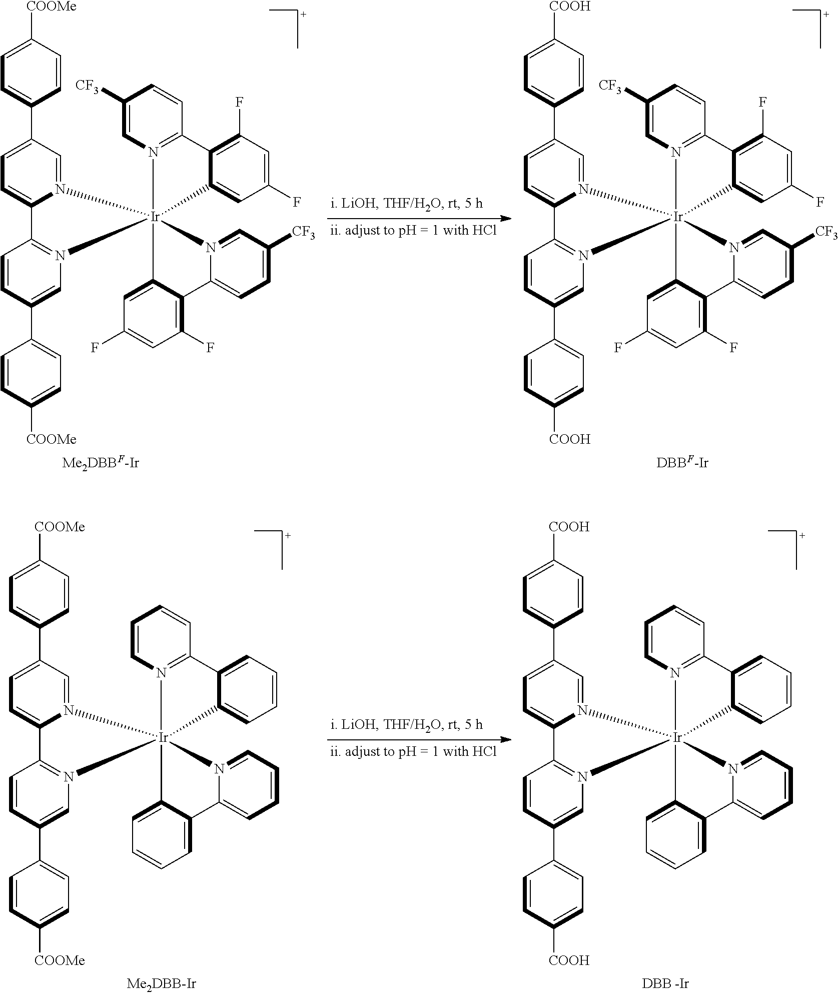

Synthesis of Hf-DBB^{F}-Ir and Hf-DBB-Ir To a 4 mL glass vial was added 0.5 mL of HfCl₄ solution (2.0 mg/mL in DMF), 0.5 mL of H₂DBB^{F}-Ir solution (4.0 mg/mL in DMF), 2.6 μL of TFA, and 2 μL of water. The reaction mixture was kept in a 70° C. oven for 24 hours. The yellow precipitate was collected by centrifugation and washed with DMF and ethanol. The yield was 61% based on Hf as determined by ICP-MS. To a 4 mL glass vial was added 0.5 mL of HfCl₄ solution (1.6 mg/mL in DMF), 0.5 mL of H₂DBB-Ir solution (6.4 mg/mL in DMF), and 100 μL of AcOH. The reaction mixture was kept in a 70° C. oven for 72 hours. The orange precipitate was collected by centrifugation and washed with DMF and ethanol. The yield was 52% based on Hf as determined by ICP-MS.

Digestion of Hf-DBB^{F}-Ir and Hf-DBB-Ir 1.0 mg Hf-DBB^{F}-Ir was dried under vacuum. The resulting solid was digested in a solution of 500 μL DMSO-d₆ and 50 μL D₃PO₄ and sonicated for 10 min. The mixture was then added to 50 μL D₂O and analyzed by ¹H NMR. The digested Hf-DBB^{F}-Ir showed all signals corresponding to H₂DBB^{F}-Ir without any other aromatic signals, which confirms the presence of only DBB^{F}-Ir ligands in Hf-DBB^{F}-Ir. 1.0 mg Hf-DBB-Ir was dried under vacuum. The resulting solid was digested in a solution of 500 μL DMSO-d₆ and 50 μL D₃PO₄ and sonicated for 10 min. The mixture was then added to 50 μL D₂O and analyzed by ¹H NMR. The digested Hf-DBB-Ir showed all signals corresponding to H₂DBB-Ir without any other aromatic signals, which confirms the presence of only DBB-Ir ligands in Hf-DBB-Ir.

^{·}OH generation with APF assay Aminophenyl fluorescein (APF, Thermo Fisher Scientific, Waltham, Massachusetts, United States of America) reacts with ^{·}OH to give bright green fluorescence (excitation/emission maxima 490/515 nm). H₂DBB^{F}_Ir, H₂DBB-Ir, Hf-DBB^{F}-Ir, and Hf-DBB-Ir were suspended in water at an equivalent concentration of 20 μM in the presence of 5 μM APF. A water solution of 5 μM APF was used as blank control. 100 μL of each suspension was added to a 96-well plate and then irradiated with X-rays at 0, 1, 2, 3, 5, or 10 Gy (RT250 X-ray generator (Philips, Andover, Massachusetts, United States of America), 250 kVp, 15 mA, 1 mm Cu filter). The fluorescence signal was immediately collected with an IVIS 200 imaging system (Xenogen, Hopkinton, Massachusetts, United States of America).

^{·}O₂ generation with SOSG assay Singlet oxygen sensor green (SOSG, Thermo Fisher Scientific, Waltham, Massachusetts, United States of America) reacts with 102 to give bright green fluorescence (excitation/emission maxima 504/525 nm). H₂DBB^{F}_Ir, H₂DBB-Ir, Hf-DBB^{F}-Ir, and Hf-DBB-Ir were suspended in water at an equivalent concentration of 20 μM in the presence of 12.5 μM SOSG. A water solution of 12.5 μM SOSG was used as blank control. 100 μL of each suspension was added to a 96-well plate and then irradiated with X-rays at 0, 1, 2, 3, 5, or 10 Gy (RT250 X-ray generator (Philips, Andover, Massachusetts, United States of America), 250 KVp, 15 mA, 1 mm Cu filter). The fluorescence signal was immediately collected with an IVIS 200 imaging system (Xenogen, Hopkinton, Massachusetts, United States of America).

O₂⁻ generation determined by BMPO assay 5-tert-butoxycarbonyl 5-methyl-1-pyrroline N-oxide (BMPO) is a nitrone spin trap, which can form distinguishable adducts with O₂⁻ (BNPO-O₂⁻) with a long half-life (t_{1/2}=23 minutes). H₂DBB^{F}_Ir, H₂DBB-Ir, Hf-DBB^{F}-Ir, and Hf-DBB-Ir were suspended in benzene at an equivalent concentration of 200 μM in the presence of 25 mM BMPO. A benzene solution of 25 mM BMPO was used as a blank control. 1 mL of each suspension was added to a 4 mL vial and then irradiated with X-ray at 5 Gy (RT250 X-ray generator (Philips, Andover, Massachusetts, United States of America), 250 kVp, 15 mA, 1 mm Cu filter). The electron paramagnetic resonance (EPR) signal was immediately collected by an X-Band ELEXSYS-II 500 EPR (Bruker, Billerica, Massachusetts, United States of America).

DNA double strand breaks The DNA double strand breaks were detected by probing phosphorylated γ-H2AX. MC38 cells were cultured in a 6-well plate at 5×10⁵/well overnight and incubated with PBS, H₂DBB-Ir, H₂DBB^{F}-Ir, Hf-DBB-Ir, or Hf-DBB^{F}-Ir at an equivalent concentration of 20 μM followed by irradiation at 0 and 2 Gy (RT250 X-ray generator (Philips, Andover, Massachusetts, United States of America), 250 kVp, 15 mA, 1 mm Cu filter). Cells were stained 2 h after irradiation with the HCS DNA damage kit (Life Technologies, Carlsbad, California, United States of America) with 1:500 dilution for flow cytometric analysis.

Clonogenic assay MC38 cells were cultured in a 6-well plate overnight and incubated with particles at a Hf concentration of 20 μM for 4 h followed by irradiation with 0, 1, 2, 4, 8 and 16 Gy (RT250 X-ray generator (Philips, Andover, Massachusetts, United States of America), 250 KVp, 15 mA, 1 mm Cu filter). The irradiated cells were trypsinized and counted immediately. 200-2000 cells were seeded in a 6-well plate and cultured with 2 mL medium for 14 days to form visible colonies, which were counted to determine the survival fraction. Once colony formation was observed, the culture medium was discarded. The plates were rinsed twice with PBS, then stained with 500 μL of 0.5% w/v crystal violet in 50% methanol/H₂O. The wells were rinsed with water three times and the colonies were counted manually. The radiation enhancement factor at 10% survival dose (REF₁₀) was calculated as the ratio of equivalent irradiation doses needed to give 10% survival rate for the PBS control group over that for the experimental group.

Cytotoxicity assay 3-(4,5-dimethylthiazol-2-yl)-5-(3-carboxymethoxy-phenyl)-2-(4-sulfo-phenyl)-2H-tetrazolium (MTS) assay (Promega, Madison, Wisconsin, United States of America) was used to evaluate cytotoxicity with X-ray irradiation. MC38 cells were seeded on 96-well plates at 1×10⁴/well and further cultured for 12 h. PBS, H₂DBB-Ir, H₂DBB^{F}-Ir, Hf-DBB-Ir, or Hf-DBB^{F}-Ir was added to the cells at an equivalent ligand dose of 0, 1, 2, 5, 10, 20, 50 and 100 μM and incubated for 4 h. The cells were then irradiated with X-rays at a dose of 2 Gy (RT250 X-ray generator (Philips, Andover, Massachusetts, United States of America), 250 KVp, 15 mA, 1 mm Cu filter). The cells were further incubated for 72 h before determining the cell viability by MTS assay.

Live/dead cell analysis The live/dead cell analysis was evaluated with cell permeable dye calcein AM and propidium iodide (PI) kit. MC38 cells were cultured in a 6-well plate at 5×10⁵/well overnight and incubated with PBS, H₂DBB-Ir, H₂DBB^{F}_Ir, Hf-DBB-Ir, or Hf-DBB^{F}-Ir at an equivalent concentration of 20 μM for 4 h by irradiation with 0 or 2 Gy (RT250 X-ray generator (Philips, Andover, Massachusetts, United States of America), 250 kVp, 15 mA, 1 mm Cu filter). The cells were then washed with PBS gently and stained with calcein AM (green) for visualization of live cells and with PI (red) for visualization of dead cells under confocal laser scanning microscopy using a confocal microscope sold under the tradename FLUOVIEW™ FV1000 (Olympus, Tokyo, Japan).

Apoptosis/necrosis Cell death analysis was evaluated with apoptotic cell death kit. MC38 cells were cultured in a 6-well plate at 5×10⁵/well overnight and incubated with PBS, H₂DBB-Ir, H₂DBB^{F}-Ir, Hf-DBB-Ir, or Hf-DBB^{F}-Ir at an equivalent concentration of 20 μM for 4 h followed by irradiation with 0 or 2 Gy (RT250 X-ray generator (Philips, Andover, Massachusetts, United States of America), 250 kVp, 15 mA, 1 mm Cu filter). 24 h later, the cells were stained according to the AlexaFluor 488 Annexin V/dead cell apoptosis kit (Life Technologies, Carlsbad, California, United States of America), imaged under CLSM and quantified using a flow cytometer sold under the tradename LSRFORTESSA™ 4-15 (BD Biosciences, San Jose, California, United States of America).

Immunogenic cell death Immunogenic cell death was examined by calreticulin (CRT) exposure. MC38 cells were cultured in a 6-well plate at 5×10⁵/well overnight and incubated with PBS, H₂DBB-Ir, H₂DBB^{F}-Ir, Hf-DBB-Ir, or Hf-DBB^{F}_Ir at an equivalent concentration of 20 μM followed by irradiation at 0 and 2 Gy (RT250 X-ray generator (Philips, Andover, Massachusetts, United States of America), 250 kVp, 15 mA, 1 mm Cu filter). The cells were then washed with PBS gently and stained with AlexaFluor 488-CRT antibody (Enzo Life Sciences, Farmingdale, New York, United States of America) with 1:100 dilution for flow cytometric analysis.

Phagocytosis C57BL/C bone-marrow-derived monocytic cells were harvested, cultured and activated. Murine granulocyte-macrophage colony-stimulating factor (GM-CSF) and IL-4 were supplied to a final concentration of 1% for 168 h and the non-adherent cells as immature DCs were harvested for following studies. Cells were incubated at under 5% C02 at 37° C. Medium was replaced every 2-3 days and cells were used after 6 to 8 days of culture. 5×10⁵ CFSE-labeled (Life Technologies, Carlsbad, California, United States of America) MC38 cells were cultured in a 6-well plate overnight and incubated with H₂DBB-Ir, H₂DBB^{F}-Ir, Hf-DBB-Ir, and Hf-DBB^{F}_Ir at an equivalent dose of 20 μM for 4 h followed by X-ray irradiation at a dose of 0 or 2 Gy (RT250 X-ray generator (Philips, Andover, Massachusetts, United States of America), 250 kVp, 15 mA, 1 mm Cu filter). 1×10⁶ PE-Cy5.5-labeled DCs were added and co-cultured with the treated MC38 cells at 37° C. for 4 h. Cells were then collected, washed twice with cold PBS, imaged by CLSM or analyzed by flow cytometry.

Immunofluorescence staining Tumors and lymph nodes were collected and subsequently frozen. Tissue sections with a thickness of 5 μm were prepared using a CM1950 cryostat (Leica Camera, Wetzler, Germany). These sections were air-dried for at least 1 h and then fixed in acetone at 20° C. for 20 min. After blocking with 20% donkey serum, the sections were incubated with individual primary antibodies against CD11b (53-6.7), F4/80 (H57-597), CD11b (53-6.7), MHC-II (53-6.7), CD86 (53-6.7), CD206 (53-6.7), and CD8a (53-6.7) overnight at 4° C., followed by incubation with dye-conjugated secondary antibodies for 1 h at r.t. After staining with DAPI for another 10 min, the sections were then washed twice with PBS and observed under SP8 LIGHTNING confocal microscope (Leica Camera, Wetzler, Germany).

In situ vaccination on syngeneic models. Synergistic tumor models, MC38, MC38-ova and Panc02 were established to evaluate the in vivo anti-cancer efficacy of nMOF-mediated in situ vaccination. For single tumor models, 5×10⁵ MC38 cells, 1×10⁶ MC38-ova cells or 1×10⁶ Panc02 cells were subcutaneously inoculated onto the right flanks of C57BL/6 mice. When the tumors reached 100-150 mm³ in volume, mice were injected intratumourally with nMOFs at a dose of 0.2 μmol Hf, CpG at a dose of 1 μg or PBS. 12 h after injection, mice were anaesthetized with 2% (v/v) isoflurane and the tumors were irradiated with 1 Gy X-ray/fraction (225 kVp, 13 mA, 0.3 mm-Cu filter) for a total of 5 daily fractions. For bilateral tumor models, 5×10⁵ MC38 cells were subcutaneously inoculated onto the right flanks as primary tumors while 2×10⁵ MC38 cells, 2×10⁵ B16F10 cells or 5×10⁵ LL2 cells were subcutaneously inoculated onto the left flanks as distant tumors of C57BL/6 mice. αPD-L1 (Clone: 10F.9G2, Catalog No. BE0101, BioXCell, Lebanon, New Hampshire, United States of America) were given every three days by intraperitoneal injection at a dose of 75 μg/mouse. The tumor sizes were measured daily with a caliper where tumor volume equals (width²×length)/2.

ELISpot assay. Tumor-specific immune responses to IFN-γ were measured in vitro by ELISpot assay (Mouse IFN-γ assay sold under the tradename ELISPOT READY-SET-GO!™; Cat. No. 88-7384-88; eBioscience, San Diego, California, United States of America). A Millipore Multiscreen HTS-IP plate (MilliporeSigma, Burlington, Massachusetts, United States of America) was coated overnight at 4° C. with anti-mouse IFN-γ capture antibody. Single-cell suspensions of splenocytes were obtained from MC38 tumor-carrying mice and seeded onto the antibody-coated plate at a concentration of 2×10⁵ cells per well. Cells were incubated with or without peptide sequence (KSPWFTTL) (SEQ ID NO: 5 for 42 h at 37° C. and then discarded. The plate was then incubated with biotin-conjugated anti-IFN-γ detection antibody at r.t. for 2 h, followed by incubation with Avidin-HRP at r.t. for 2 h. 3-amino-9-ethylcarbazole substrate solution (Sigma, St. Louis, Missouri, United States of America, Cat. AEC101) was added for cytokine spot detection. Spots were imaged and quantified with an analyzer sold under the tradename IMMUNOSPOT™ (Cellular Technology Ltd, Shaker Heights, Ohio, United States of America).

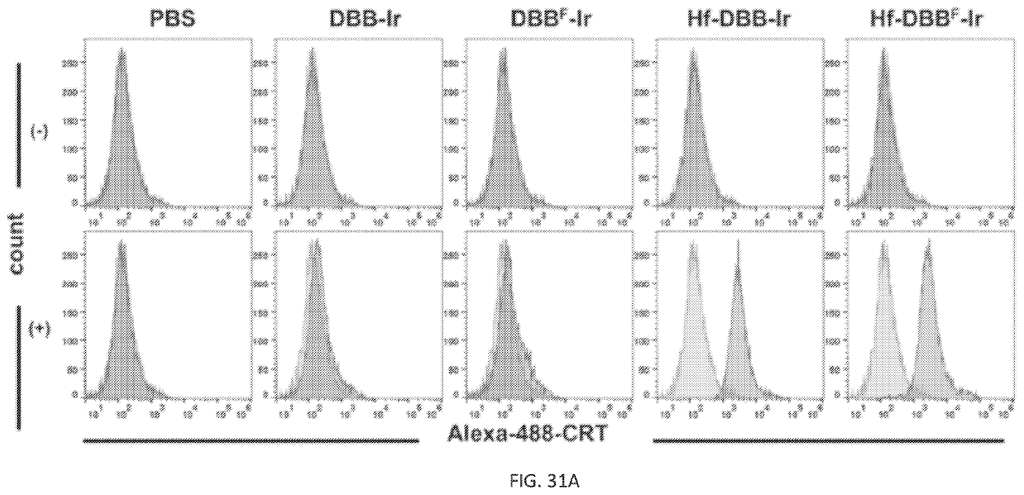

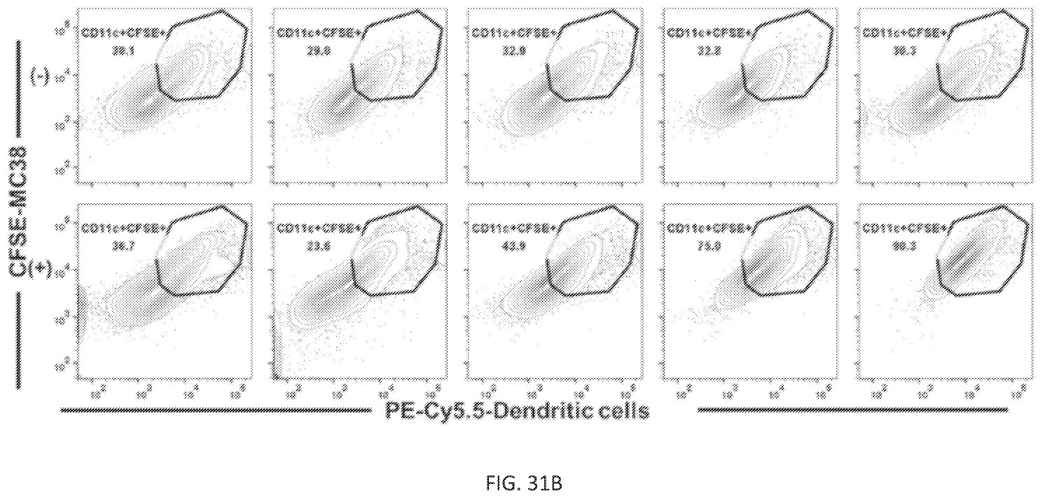

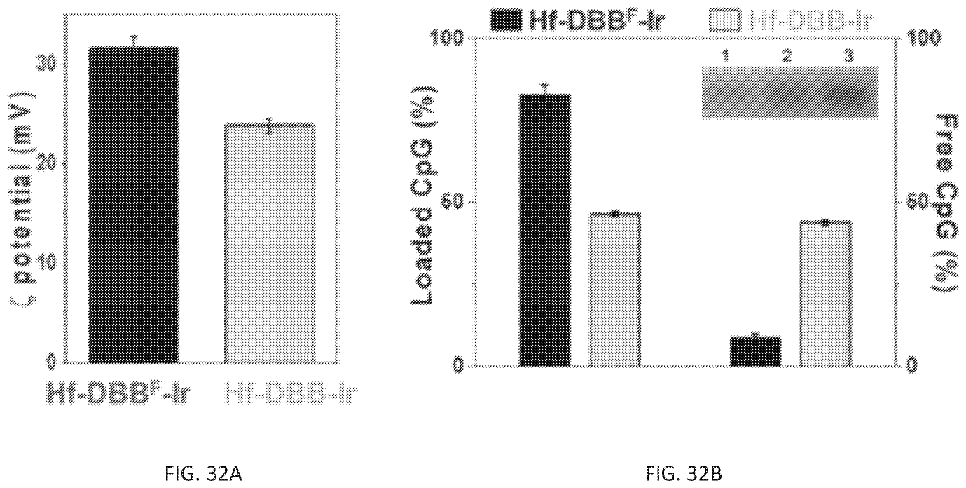

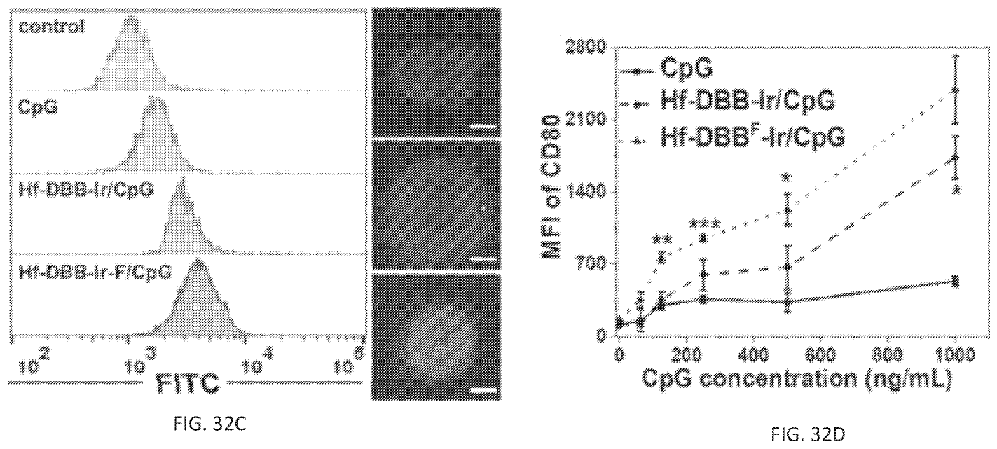

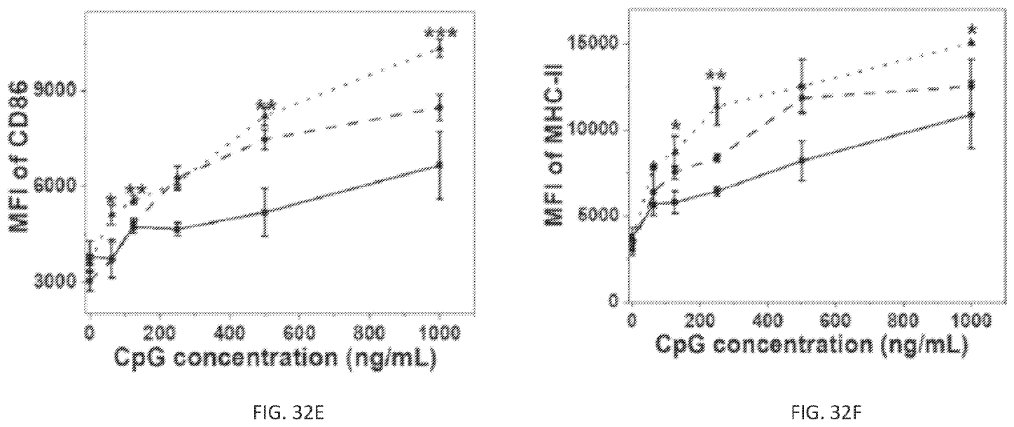

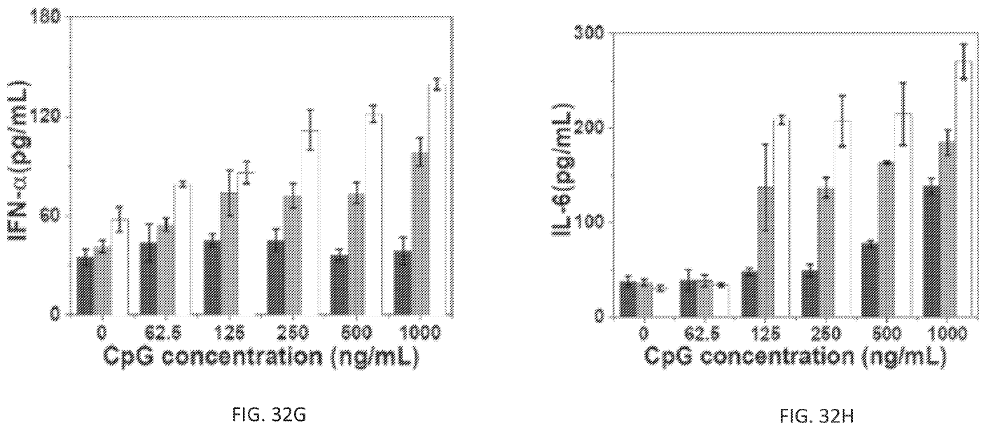

Lymphocyte profiling. Tumors and lymph nodes were harvested, treated with 1 mg/ml collagenase I (Gibco Laboratories, Gaithersburg, Maryland, United States of America) for 1 h at 37° C. Cells were filtered through nylon mesh filters with size of 40 μm and washed with PBS. Tumor-draining lymph nodes were collected and directly ground through the cell strainers. The single-cell suspension was incubated with anti-CD16/32 (clone 93) to reduce nonspecific binding to FcRs. Cells were further stained with the following fluorochrome-conjugated antibodies: CD45 (30-F11), CD3a (145-2C11), CD4 (GK1.5), CD8a (53-6.7), Nkp46 (29A1.4), F4/80 (BM8), CD11b (M1/70), Gr-1 (RB6-8C5), MHC-II (AF6-120), CD80 (16-10A1), CD86 (GL1), CD206 (C068C2), CD44 (IM7), CD62L (MEL-14), H-2K^{b} SIINFEKL (SEQ ID NO: 3) (25-D1.16), PI, and yellow-fluorescent reactive dye (CD45 from BD Bioscience (San Jose, California, United States of America), CD206 and CD62L from Biolegend (San Diego, California, United States of America), others from eBioscience (San Diego, California, United States of America)). Antibodies were used with the dilution of 1:200. Representative gating strategies for different immune cells are shown in Supplementary FIG. 31-32. A flow cytometer sold under the tradename LSRFORTESSA™ 4-15 (BD Biosciences, San Jose, California, United States of America) was used for cell acquisition and data analysis was carried out with FlowJo software (Tree Star, Ashland, Oregon, United States of America).

Adoptive OT-I T cells transfer 2×10⁶ MC38-ova cells were injected subcutaneously onto the right flanks of C57BL/6 Rag2^{-/-} mouse. 14 days later, mice were intratumorally injected with Hf-DBB^{F}-Ir at a dose of 0.2 μmol Hf with or without CpG at a dose of 1 μg followed by 1 Gy X-ray/fraction for a total of 5 daily fractions. 2 days post the first irradiation, spleen and lymph nodes were isolated from OT-I mice and CD8⁺ T cells were negatively sorted using mouse CD8⁺ T Cell Isolation Kit (Miltenyi Biotec, Bergisch Gladbach, Germany), 1×10⁶ CD8⁺ T cells were intravenously injected into MC38-ova-bearing Rag2^{-/-} mice. The tumor sizes were measured daily with a caliper where tumor volume equals (width²×length)/2.

Statistical analysis. Group sizes (n≥5) were chosen to ensure proper statistical ANOVA analysis for efficacy studies. Student's t-tests were used to determine if the variance between groups is similar. Statistical analysis was performed using OriginPro (OriginLab Corp., Northampton, Massachusetts, United States of America). Statistical significant was calculated using two-tailed Student's t-tests and defined as * P<0.05, ** P<0.01, *** P<0.001. Animal experiments were not performed in a blinded fashion and are represented as mean±SD. The immune analysis was performed in a blinded fashion and are represented as median±SD.

### Example 10

### Synthesis and Characterization of Hf-DBB^{F}-Ir and Hf-DBB-Ir nMOFs

According to one aspect of the presently disclosed subject matter is described a novel strategy of using nanoscale metal-organic frameworks (nMOFs) for personalized cancer vaccination via X-ray activated generation of DAMPs and tumor antigens and efficient delivery of CpGs to APCs as PAMPs. Cationic nMOFs were designed through molecular engineering to release DAMPs and tumor antigens via X-ray activated RT-RDT and to deliver CpGs via electrostatic interactions. The in situ vaccination afforded by nMOFs effectively expand cytotoxic T cells in tumor-draining lymph nodes to reinvigorate the adaptive immune system for tumor regression. See FIG. 23. The local therapeutic effects of the nMOF-based in situ vaccines were extended to distant tumors by combination treatment with an anti-PD-L1 antibody (αPD-L1) to afford an 83.3% cure rate on an MC38 colorectal cancer model.

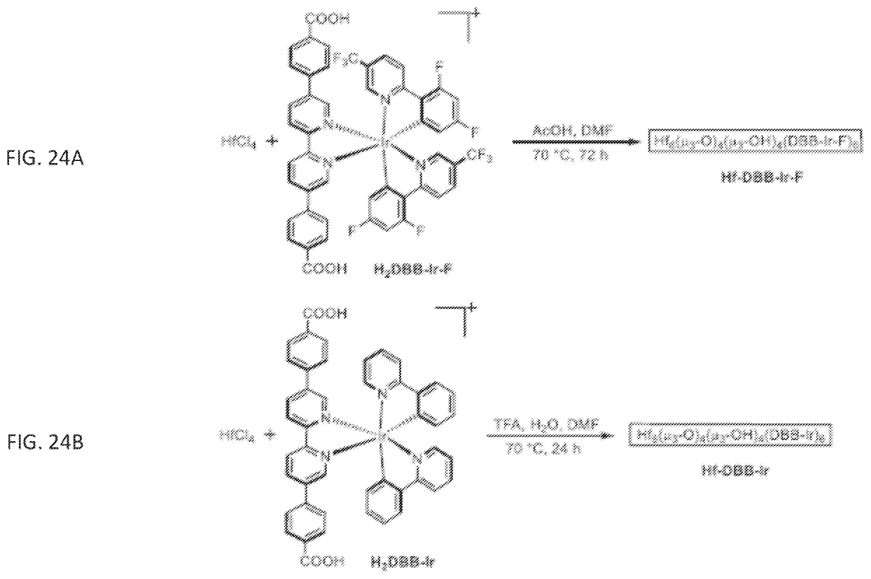

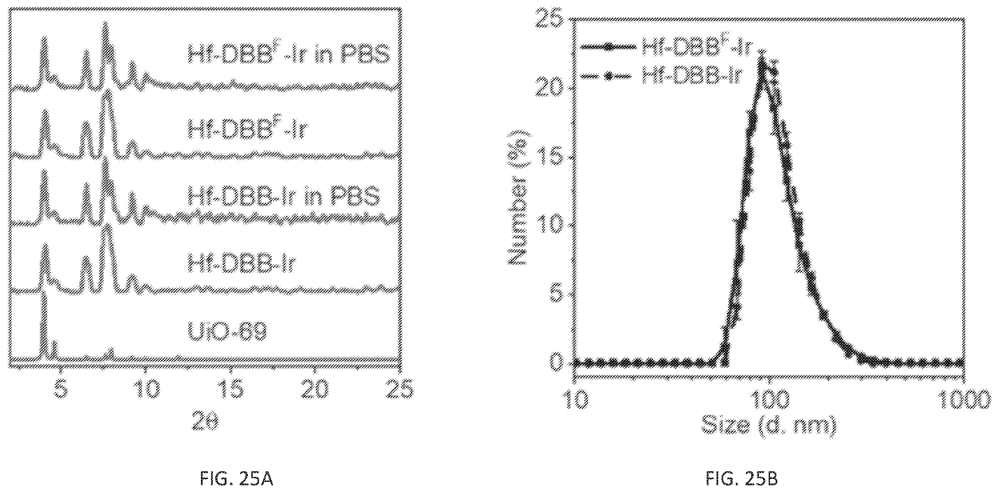

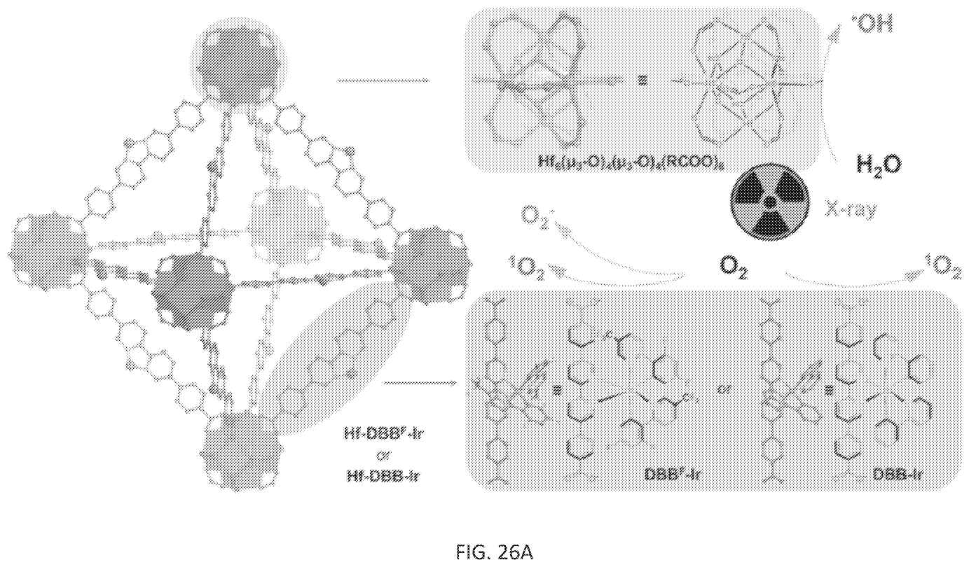

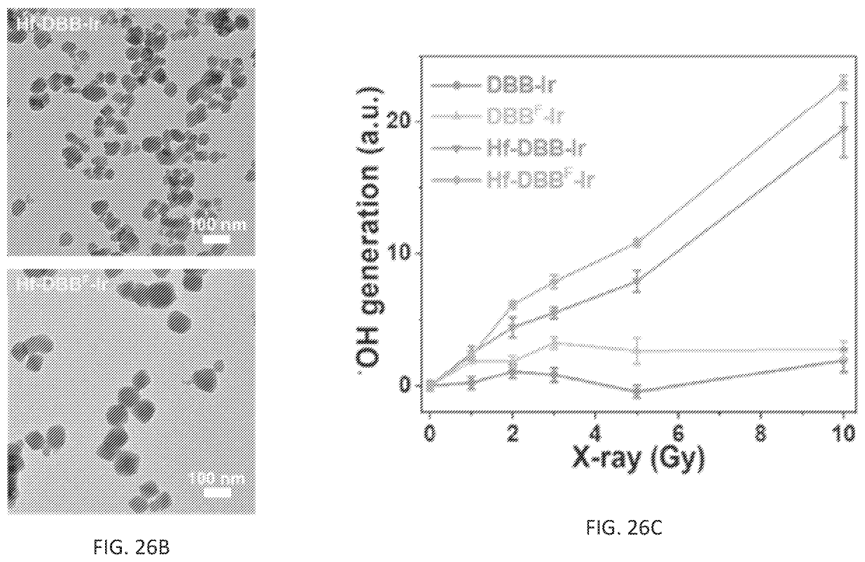

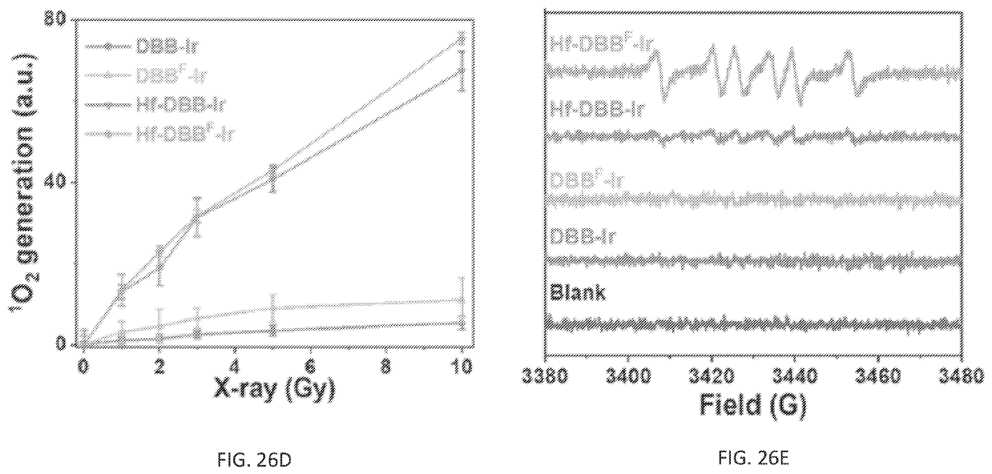

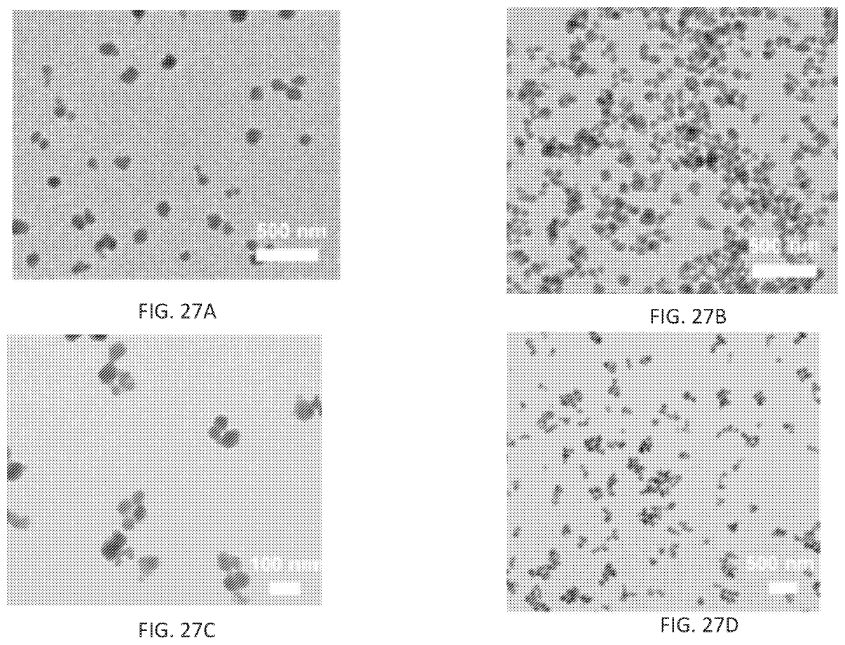

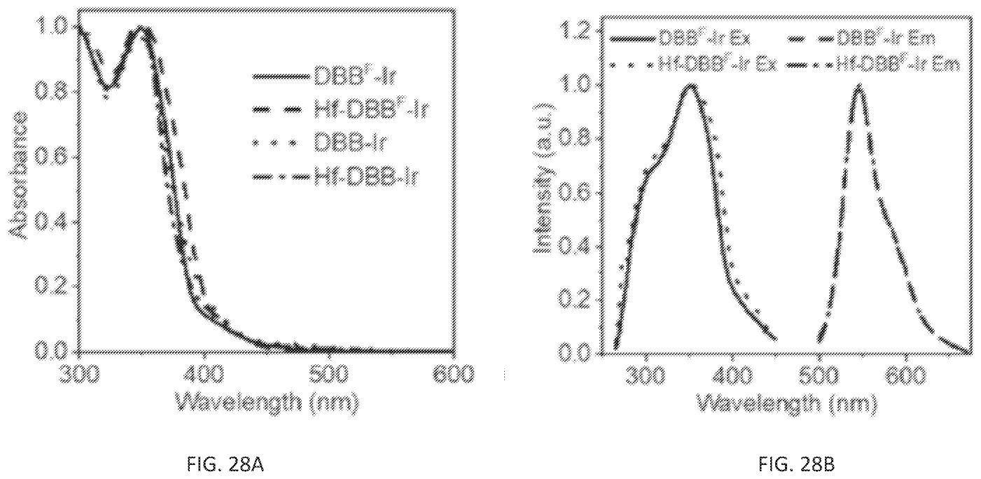

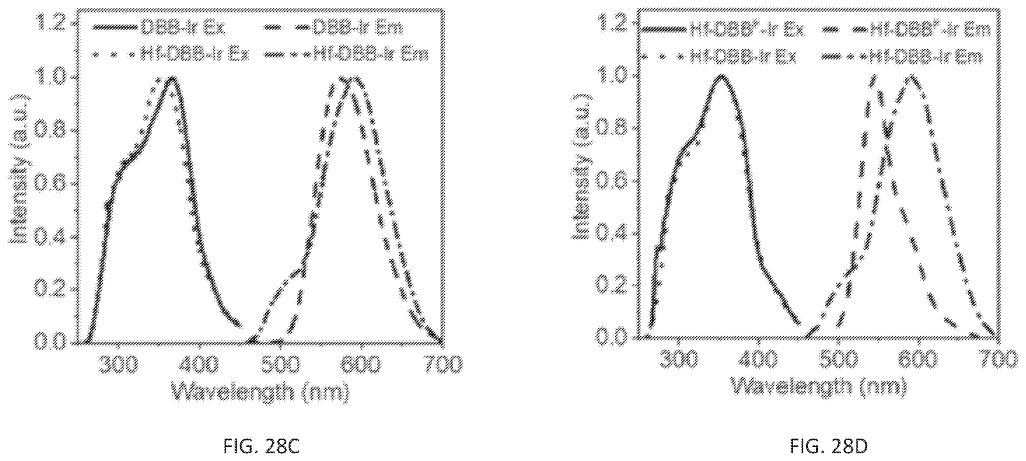

More particularly, to generate DAMPs and tumor antigens through RT-RDT and deliver PAMPs with high CpG loading, two positively charged nMOFs were designed: Hf-DBB^{F}-Ir and Hf-DBB-Ir, with high-Z metal Hf₆ secondary building units (SBUs) and photosensitizing DBB^{F}-Ir and DBB-Ir ligands, respectively. See Scheme 1, above and FIGS. 24A and 24B. Hf-DBB^{F}-Ir and Hf-DBB-Ir nMOFs possessed UiO-like structures with a formula of Hf₆(α₃-O)₄(μ₃-OH)₄L₆, where L=DBB^{F}-Ir or DBB-Ir. See FIG. 25A. Hf-DBB^{F}-Ir and Hf-DBB-Ir exhibited spherical to octahedral morphologies with diameters of ˜100 nm, as revealed by transmission electron microscopy (TEM) imaging (see FIGS. 26B and 27A-27D) and dynamic light scattering (DLS) measurements. See FIG. 25B. The photosensitizing characteristics of Hf-DBB^{F}-Ir and Hf-DBB-Ir were confirmed by UV-Vis absorption and luminescence spectroscopy, where Hf-DBB^{F}-Ir and Hf-DBB-Ir showed similar absorbance and luminescence to those of DBB^{F}-Ir and DBB-Ir, respectively. See FIGS. 28A-28D.

### Example 11

### Reactive Oxygen Species, DAMPs and In Vitro Immunogenicity

Detection of Reactive Oxygen Species: Without being bound to any one theory, it is believed that photosensitizing Hf-DBB^{F}-Ir and Hf-DBB-Ir could generate multiple reactive oxygen species (ROS) upon X-ray irradiation, including hydroxy radical (^{·}OH) through water radiolysis of Hf₆ SBUs, and singlet oxygen (¹O₂) and superoxide anion (O₂⁻) through excitation of photosensitizing ligands. See FIG. 26A. As quantified by APF and SOSG assays, both Hf-DBB^{F}-Ir plus X-ray irradiation [denoted Hf-DBB^{F}_Ir(+)] and Hf-DBB-Ir(+) exhibited significantly enhanced ^{·}OH and ¹O₂ generation in comparison to their ligand controls. See FIGS. 26C and 26D. However, only Hf-DBB^{F}-Ir(+) displayed efficient O₂⁻ generation as determined by BMPO assay, which is ascribed to the higher reduction potential of DBB^{F}-Ir than DBB-Ir. See FIG. 26E.

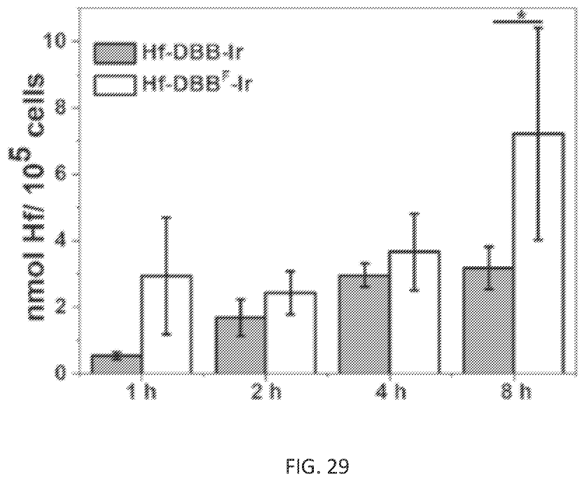

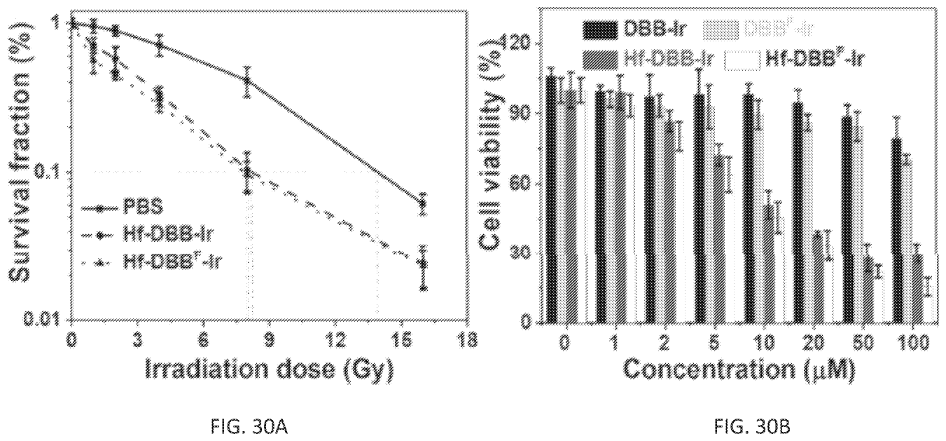

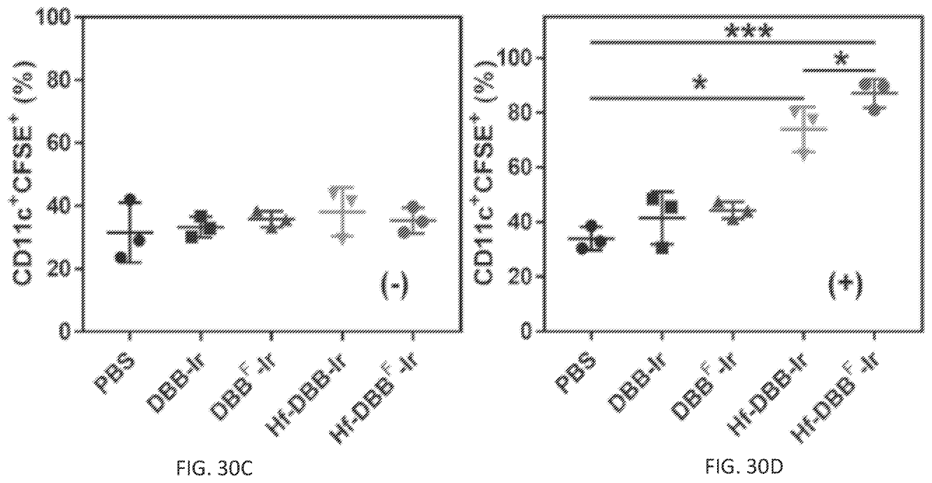

Generation of Reactive Oxygen Species in vitro: The uptake of Hf-DBB-Ir and Hf-DBB^{F}-Ir by MC38 cells was evaluated. Inductively coupled plasma-mass spectrometry (ICP-MS) studies showed the two nMOFs reached similar intracellular Hf levels after 4 h incubation. See FIG. 29. Then, in vitro ¹O₂ and O₂⁻ generation was probed by SOSG and superoxide assay kits, respectively. Both Hf-DBB-Ir(+) and Hf-DBB^{F}-Ir(+) induced strong green fluorescence, indicating significant ¹O₂ generation. However, only Hf-DBB^{F}-Ir(+) exhibited strong red fluorescence, indicating the generation of O₂⁻ through Hf-DBB^{F}-Ir-mediated RT-RDT process. See FIG. 30A. To confirm the RT effect, ^{·}OH induced DNA double-strand breaks (DSBs) were quantified by flow cytometric analysis of phosphorylated γ-H2AX in cells treated with PBS, ligands or nMOFs with or without X-ray. Interestingly, 2 h after irradiation, significantly higher red γ-H2AX fluorescence was observed in the group treated with Hf-DBB^{F}-Ir(+) than Hf-DBB-Ir(+), likely due to the biotransformation O₂⁻ to ^{·}OH by superoxidase dismutase. No fluorescence was observed in the groups without X-ray irradiation or without nMOF incubation.

Release of DAMPs and in vitro immunogenicity: To test the hypothesis that the myriad of ROSs generated by Hf-DBB^{F}-Ir damages cancer cells more effectively than other treatments, Hf-DBB^{F}-Ir mediated cell damage and DAMP generation was evaluated Clonogenic assays showed that Hf-DBB^{F}-Ir(+) slightly outperformed Hf-DBB-Ir(+) with an REF₁₀ value of 1.75 vs 1.68. See FIG. 30B. MTS assays further showed that Hf-DBB^{F}-Ir(+) exhibited higher cytotoxicity than Hf-DBB-Ir(+) with IC₅₀ values of 4.28±1.15 μM and 7.85±2.41 μM, respectively, at 2 Gy. See FIG. 30C. A greater level of cell death was also observed for Hf-DBB^{F}-Ir(+) by live/dead cell imaging and apoptotic cell quantification by CLSM and flow cytometry. See FIG. 30D. These results indicate a stronger cell killing effect by Hf-DBB^{F}-Ir(+) via the RT-RDT process.

Next, the generation of DAMPs from nMOF-mediated RT-RDT was investigated by examining immunogenic cell death (ICD) of tumor cells and phagocytosis of dying tumor cells and their apoptotic debris by APCs. In the ICD process, calreticulin (CRT) is translocated to cell membrane as an “eat-me” signal which is recognized by macrophages and DCs to engulf dying tumor cells and their apoptotic debris. Flow cytometric quantification revealed that Hf-DBB^{F}-Ir(+) treated cells exhibited higher CRT fluorescence, suggesting that Hf-DBB^{F}-Ir(+) induced stronger ICD with higher cytotoxicity. See FIG. 31A.

To assess the impact of nMOF-mediated RT-RDT on antigen processing by and immune activation of APCs, DCs differentiated from bone marrow cells were co-cultured with CFSE-labeled MC38 cells treated with PBS, DBB-Ir, DBB^{F}-Ir, Hf-DBB-Ir, or Hf-DBB^{F}-Ir with or without X-ray irradiation. Flow cytometry showed that Hf-DBB^{F}-Ir(+) treatment induced significantly higher population of PE-Cy5.5 conjugated CD11c-labelled DCs with phagocytosed CFSE-labelled MC38 cells than other treatment groups, indicating enhanced immune stimulation mediated by cationic nMOFs. See FIG. 31B. CLSM imaging confirmed that more CD11c⁺DCs phagocytosed Hf-DBB^{F}_Ir(+) treated CFSE⁺ MC38 cells.

In vitro delivery of PAMPs: Without being bound to any one theory, it was rationalized that fluorination of the DBB^{F} ligand in Hf-DBB^{F}-Ir could introduce electron-withdrawing effects to increase the surface charge for more efficient delivery of CpG. Hf-DBB^{F}-Ir and Hf-DBB-Ir exhibited ζ-potential values of 31.6±1.2 mV and 23.8±0.8 mV, respectively, confirming a more cationic skeleton of Hf-DBB^{F}-Ir for electrostatic adsorption of anionic CpG. See FIG. 32A. 1 mg of CpG was incubated in 20 mL PBS solution of Hf-DBB^{F}-Ir or Hf-DBB-Ir with a Hf concentration of 10 mM for 10 mins. After centrifugation, DNA gel electrophoresis showed the adsorption of 82.7% CpG onto Hf-DBB^{F}-Ir and 46.5% CpG onto Hf-DBB-Ir, with 8.6% and 43.8% of CpG remaining in the corresponding supernatants as quantified by NanoDrop spectrophotometry. See FIG. 32B. CpG internalization by DCs was examined next. Flow cytometry and CLSM imaging showed that Hf-DBB^{F}-Ir delivered the highest amount of CpG to DCs after they were cultured with FITC-labeled free CpG, Hf-DBB-Ir@CpG or Hf-DBB^{F}-Ir@CpG. See FIG. 32C. These results confirm the superior ability of Hf-DBB^{F}-Ir in delivering CpG as PAMPs to APCs.

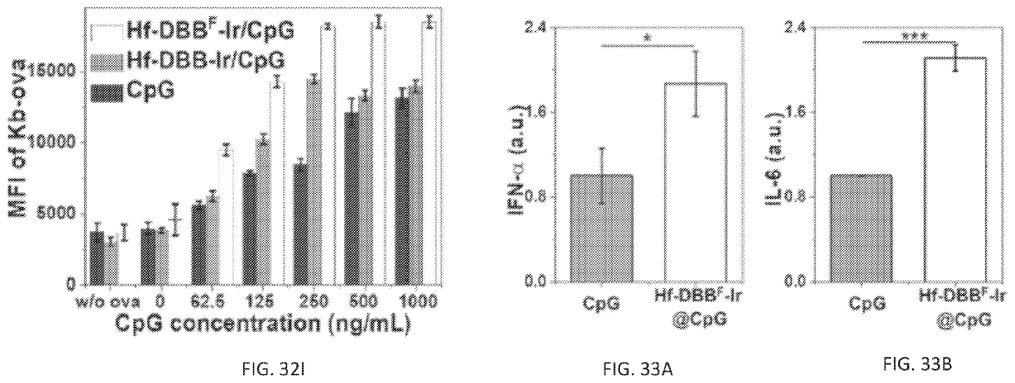

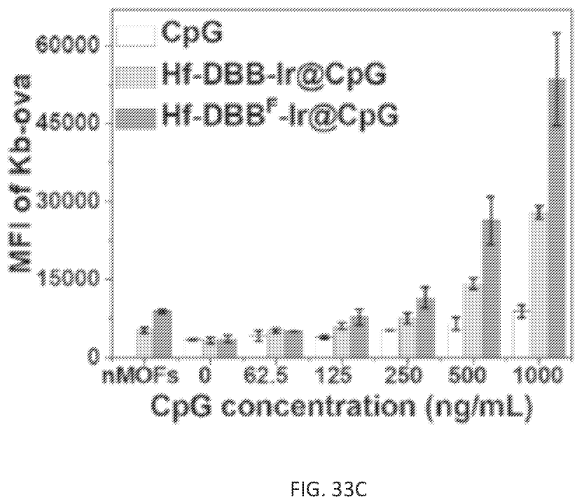

In vitro DC maturation: To evaluate the effects of CpG delivery on DC maturation, bone marrow derived DCs were incubated with CpG, Hf-DBB-Ir@CpG or Hf-DBB^{F}_Ir@CpG at CpG concentrations of 0, 62.5, 125, 250, 500 and 1000 ng/mL for 60 h. The cells were harvested and stained for the detection of DCs maturation markers, including MHC-II and co-stimulatory molecules CD80 and CD86. The supernatants were also collected and assayed for the presence of cytokines interferon-alpha (IFN-α) and interleukin-6 (IL-6). Both Hf-DBB-Ir@CpG and Hf-DBB^{F}-Ir@CpG effectively promoted DC maturation with increased MFI signals of CD80 (see FIG. 32D), CD86 (see FIG. 32E) and MHC-II compared to free anionic CpG. See FIG. 32F. Hf-DBB^{F}-Ir@CpG outperformed Hf-DBB-Ir@CpG in the upregulation of CD80, CD86, and MHC-II signals as a result of its more effective CpG delivery. Only cationic nMOF-delivered CpG showed elevated IFN-α levels while free CpG completely had no effect. See FIG. 32G. Moreover, DCs treated with free CpG excreted IL-6 only in high CpG concentrations, while treated with nMOFs/CpG excreted IL-6 at low CpG concentrations. See FIG. 32H. qPCR of IL-6 and IFN-α expression confirmed that Hf-DBB^{F}-Ir more efficiently delivered CpG as PAMPs to activate DCs. See FIGS. 33A and 33B. To directly demonstrate the enhanced antigen presentation property of DCs after Hf-DBB^{F}-Ir@CpG stimulation, MC38 cells transfected with ovalbumin antigen (OVA, cell line denoted as MC38-ova) were cultured with CpG, Hf-DBB-Ir@CpG or Hf-DBB^{F}-Ir@CpG stimulated DCs in a 3:1 ratio. Tumor antigen uptake and presentation was examined by detecting the expression of H-2K^{b}-SIINFEKL (SEQ ID NO: 3) complex (Kb-ova) on DCs surface. Hf-DBB^{F}-Ir@CpG outperformed Hf-DBB-Ir@CpG and free CpG on promoting antigen uptake and presentation by DCs (see FIGS. 32I and 33C), likely as a result of more efficient delivery of PAMPs and antigen presentation.

### Example 12

### In Situ Cancer Vaccines

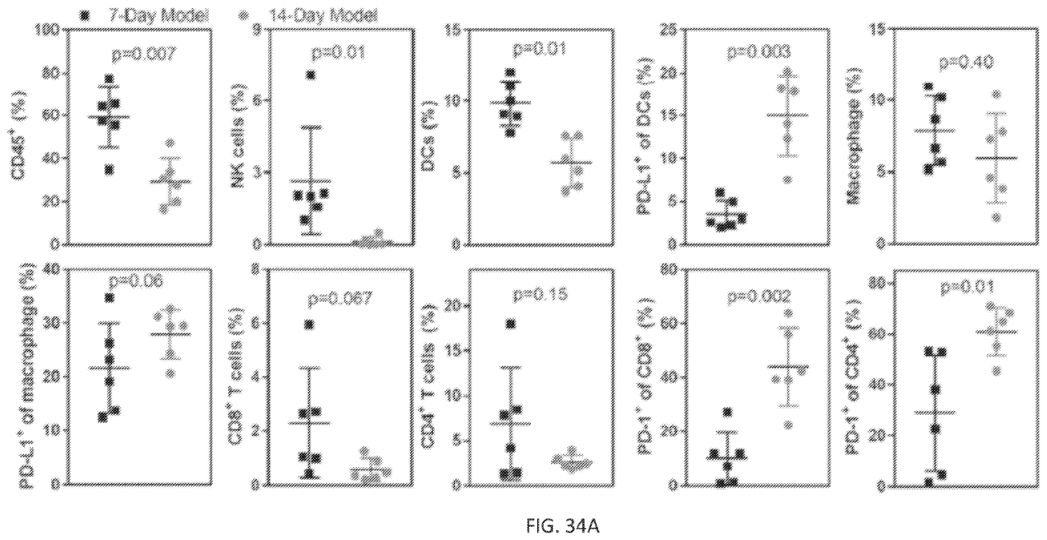

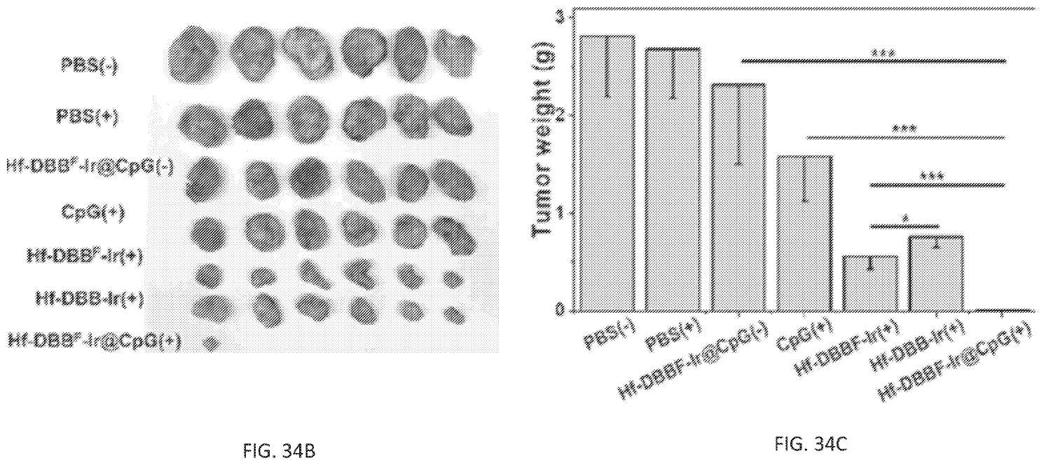

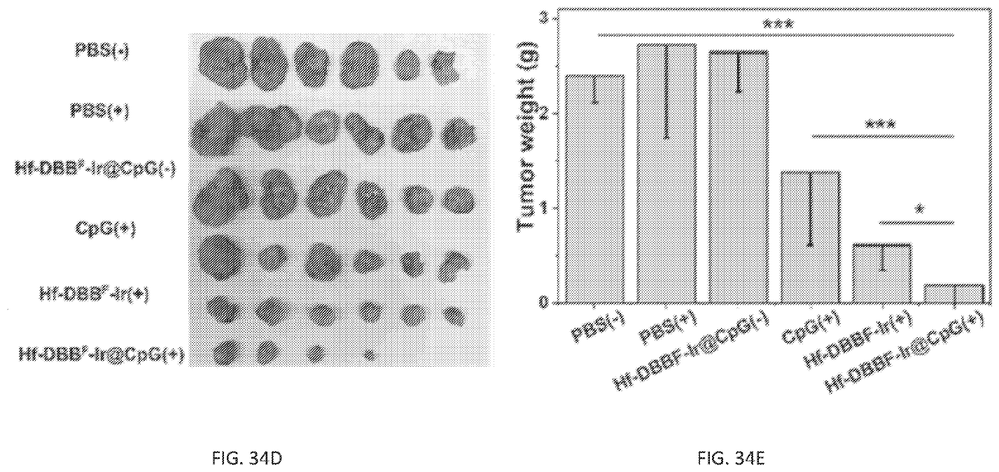

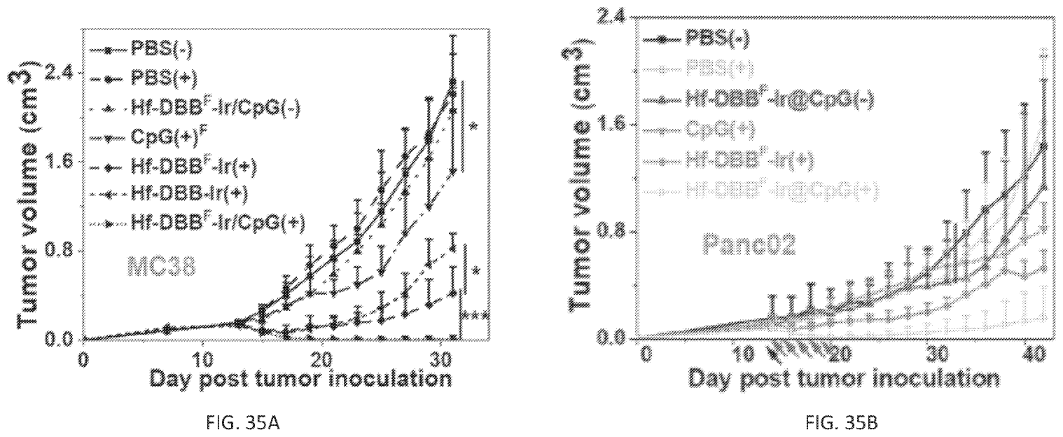

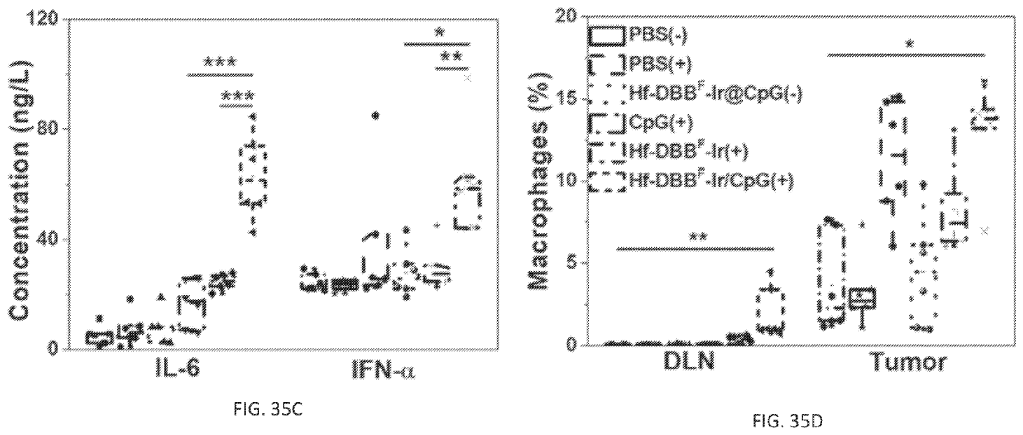

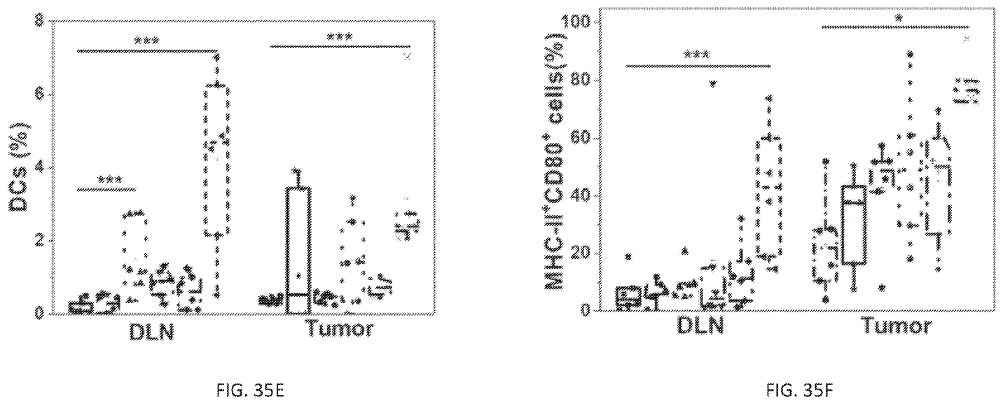

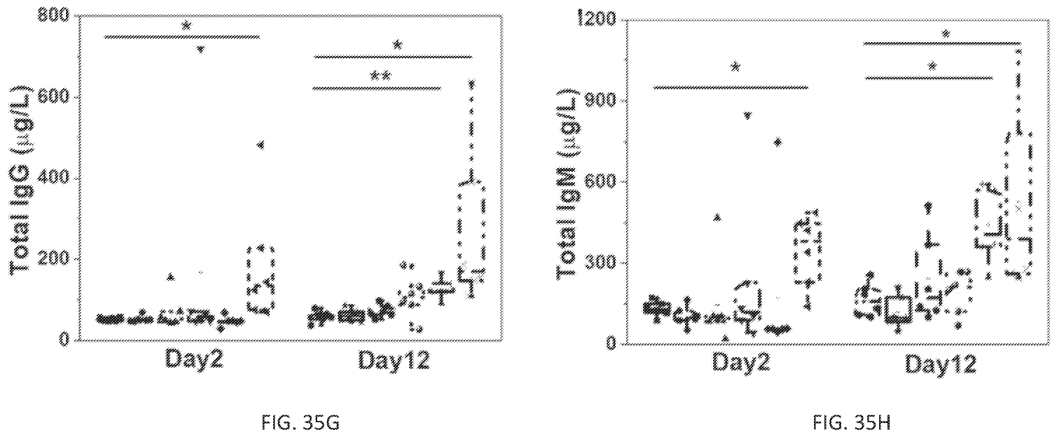

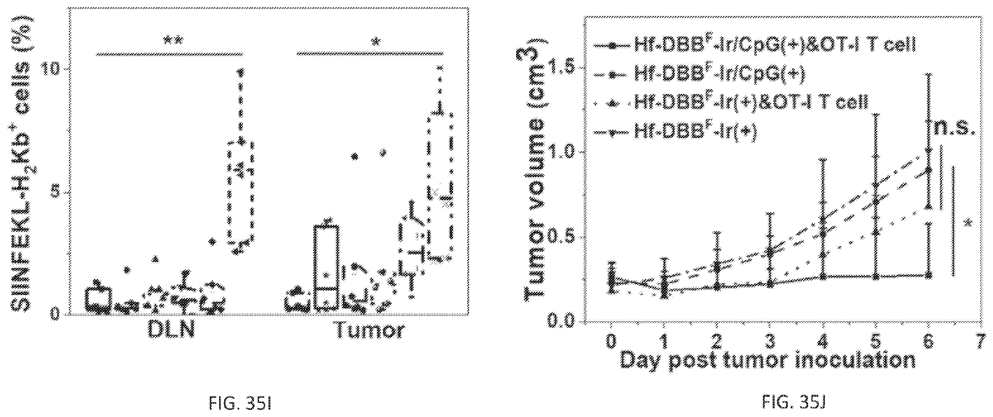

X-ray triggered in situ cancer vaccines: The local anti-cancer effect of Hf-DBB^{F}_Ir@CpG(+) was investigated as an in situ cancer vaccine. Intravenous injection of 2 mol DBB^{F}-Ir or Hf-DBB^{F}-Ir biweekly for a total of 4 doses did not cause toxicity on C57BL/6 mice as judged from steady bodyweight gains. Next, a T-cell excluded murine colorectal model MC38 on C57BL/6c mice by subcutaneous injection of 5×10⁵ MC38 cells on right flanks. In previous studies, subcutaneous MC38 tumors were established by inoculating 2×10⁶ MC38 cells and reached 100-150 mm³ in sizes at day 7 before the commencement of treatments. In contrast, when inoculated with fewer cells, MC38 tumors grew to 100-150 mm³ in 14 days and showed much more immunosuppressive tumor microenvironments than the 7-day model. See FIG. 34A. PBS, Hf-DBB-Ir, Hf-DBB^{F}-Ir, or Hf-DBB^{F}-Ir@CpG was injected intratumorally at a Hf dose of 0.2 μmol and/or CpG dose of 1 μg. 12 h later, the tumors were irradiated with 1Gy of X-ray (225 kVp, 13 mA, 1 Gy) and followed by four more daily irradiation of X-ray (1 Gy). Hf-DBB^{F}-Ir(+) outperformed Hf-DBB-Ir(+) with a tumor growth inhibition index (TGI) of 81.9% vs 64.7%, suggesting more efficient release of DAMPs by Hf-DBB^{F}-Ir-mediated RT-RDT in vivo. Hf-DBB^{F}-Ir@CpG(+) showed enhanced tumor regression over CpG(+) (TGI of 99.6% vs 34.8%) or Hf-DBB^{F}-Ir(+) on Day 31, indicating the synergy of DAMPs released by nMOF-mediated RT-RDT and PAMPs delivered by cationic nMOFs. See FIG. 35A. See also Table 4, below. The anti-cancer efficacy was confirmed by optical images and averaged weights of excised tumors on Day 31. See FIGS. 34B and 34C. Immunofluorescence of terminal deoxynucleotidyl transferase dUTP nick end labeling (TUNEL) and H&E staining indicated significant apoptosis of tumor cells with Hf-DBB^{F}-Ir@CpG(+) treatment No systemic toxicity was observed for all treatment groups. The antitumor activity was also evaluated on a murine pancreatic cancer model, Panc02, on C57BL/6c mice with high radioresistance and poor immunogenicity. Hf-DBB^{F}-Ir@CpG(+) afforded superior tumor growth inhibition over other groups (see FIGS. 34D, 34E, 35B, and Table 4), suggesting the potential of using Hf-DBB^{F}-Ir@CpG(+) as in situ cancer vaccine on a broad spectrum of cancers with varied immunogenicity.

TABLE 4
Tumor growth inhibition indices (TGIs) of MC38 and
Panc02 tumor models with different treatments.

| TGI (%) | MC38 | Panc02 |
|---|---|---|
| PBS(+) | 4.8 | 0.9 |
| CpG(+) | 34.8 | 54.8 |
| Hf-DBB-Ir(+) | 64.7 | — |
| Hf-DBB^{F}-Ir(+) | 81.9 | 69.4 |
| Hf-DBB^{F}-Ir/CpG(−) | 11.3 | 24.1 |
| Hf-DBB^{F}-Ir/CpG(+) | 99.6 | 89.1 |

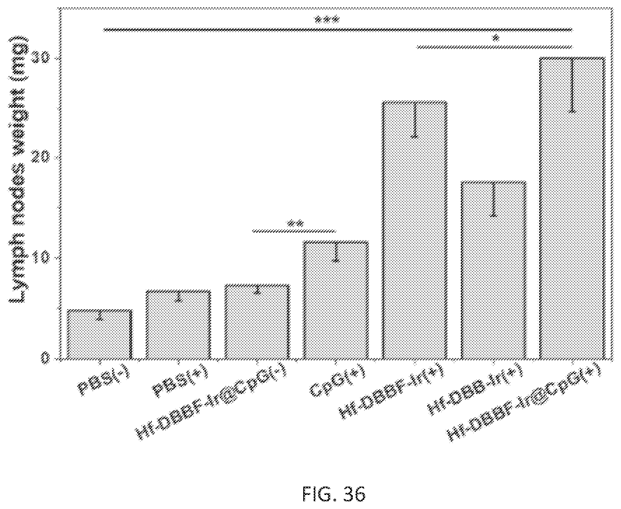

Innate immunity after in situ cancer vaccination: Plasma IL-6 and IFN-α concentrations were assayed by ELISA and gene expression was determined in tumors and tumor-draining lymph nodes (DLNs) by qPCR 24 h post treatment to evaluate the innate immune response. Hf-DBB^{F}-Ir@CpG(+) treatment showed significantly elevated levels of plasma and intratumoral IL-6 and IFN-α over CpG(+) or Hf-DBB^{F}_Ir(+) treatment. See FIG. 35C. Furthermore, flow cytometry and CLSM studies showed significant increases of tumor- and DLN-infiltrating APCs, including macrophages (see FIG. 35D) and DCs (see FIG. 35E), in the Hf-DBB^{F}-Ir@CpG(+) treatment group, which indicates the synergistic effect of DAMPs and tumor antigens released by nMOF-mediated RT-RDT and PAMPs delivered by cationic nMOFs. DC maturation promoted by Hf-DBB^{F}-Ir@CpG(+) was further demonstrated with elevated expression of MHC-II and costimulatory CD80 molecules. See FIG. 35F. Elevations of total IgG (see FIG. 35G) and IgM (see FIG. 35H) in plasma 2 and 12 days after Hf-DBB^{F}_Ir@CpG(+) treatment suggest effective promotion of humoral immunity mediated by B cells. As IgM can bind and activate the complement system to promote the opsonization and degradation of antigens and antigen presentation by phagocytes, the increased levels of plasma IgG and IgM results imply an important role of B cells in promoting antigen presentation after in situ vaccination. The expression of Kb-ova complex (SIINFEKL (SEQ ID NO: 3)-H₂Kb gated from CD45⁺ cells) was significantly upregulated post Hf-DBB^{F}-Ir@CpG(+) treatment on the MC38-ova model, confirming the antigen presentation process. See FIG. 35I. Hf-DBB^{F}-Ir@CpG(+) group also exhibited enlarged DLNs (see FIG. 36), suggesting T cell expansion in DLNs. The increased expression of Ki67 in DLNs by CLSM supported T cell expansion in DLNs following Hf-DBB^{F}-Ir@CpG(+) treatment. Finally, MC38-ova tumors were established on immuno-deficient Rag2^{-/-} mice and then treated with Hf-DBB^{F}-Ir(+) or Hf-DBB^{F}_Ir@CpG(+) plus adoptive transfer of OT-I T cells. Mice treated with Hf-DBB^{F}_Ir@CpG(+) plus OT-I T cell transfer showed more effective tumor suppression than either Hf-DBB^{F}-Ir(+) plus OT-I T cell transfer or Hf-DBB^{F}-Ir@CpG(+) alone (see FIG. 35J), supporting an effective antigen presentation process after Hf-DBB^{F}_Ir@CpG(+) treatment as an in situ cancer vaccine. Interestingly, macrophage repolarization with an increased ratio of pro-inflammatory M1 subtype to anti-inflammatory (tumor-promoting) M2 subtype was observed following Hf-DBB^{F}_Ir@CpG(+) treatment.

### Example 13

### Abscopal Effect

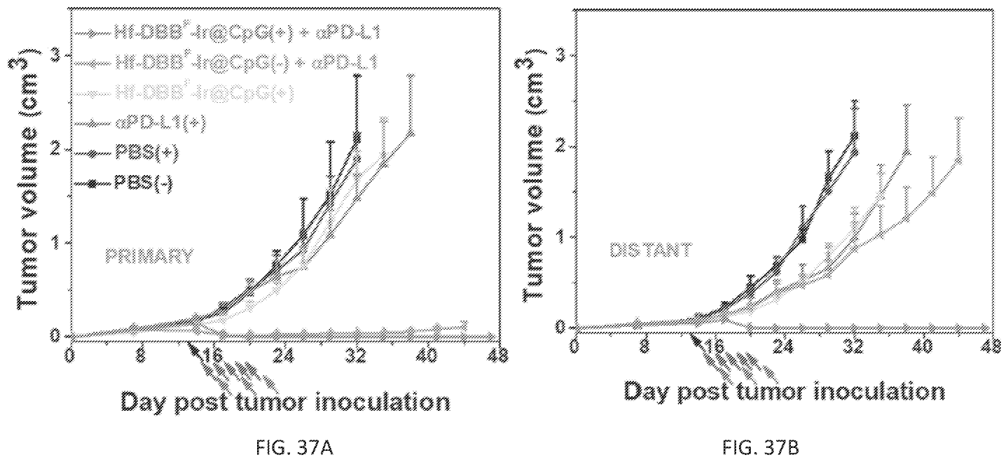

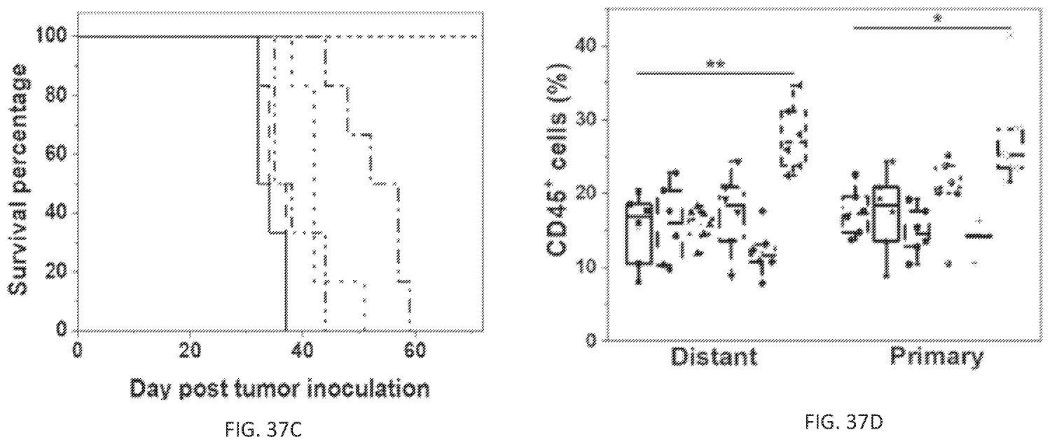

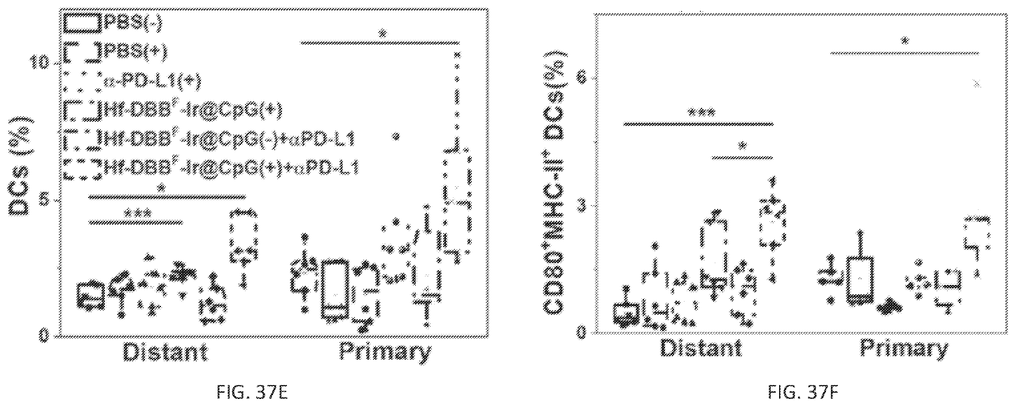

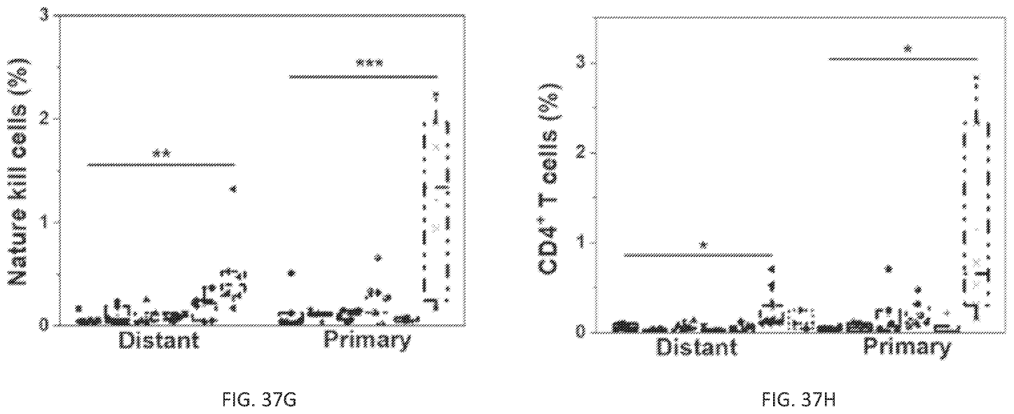

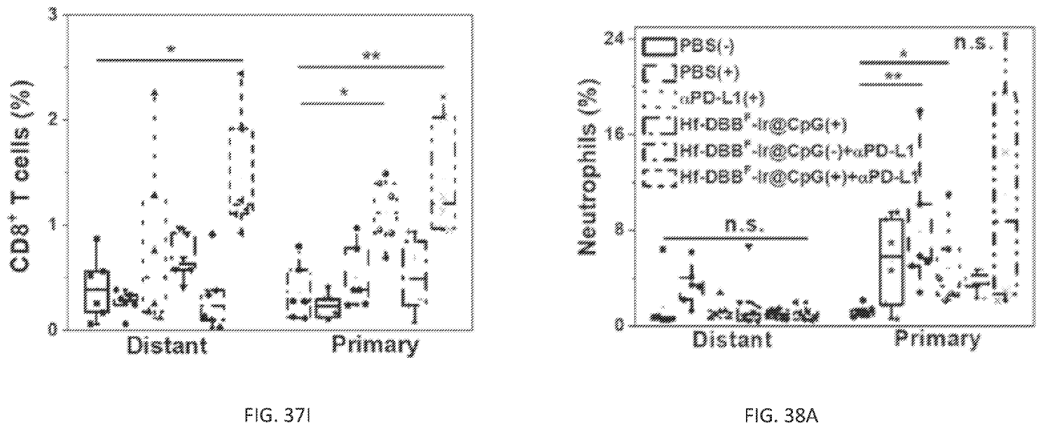

A bilateral model of MC38 was then established to assess the systemic anticancer efficacy of Hf-DBB^{F}-Ir@CpG(+) in combination with anti-PD-L1 (αPD-L1) antibody. Hf-DBB^{F}-Ir@CpG was intratumorally injected into primary tumors at a dose of 0.2 mol Hf and 1 μg CpG 14 days post tumor inoculation, with daily X-ray irradiation at a dose of 1 Gy/fraction beginning on day 15 for a total of 5 fractions. 75 μg of αPD-L1 was administered every three days by intraperitoneal injection for a total of 3 doses. Without αPD-L1, Hf-DBB^{F}-Ir@CpG(+) almost eradicated primary tumors but only moderately delayed progression of distant tumors. In stark contrast, the combination of Hf-DBB-Ir-F@CpG(+) and αPD-L1 significantly regressed both primary and distant tumors with a cure rate of 83.3%. This result indicates a strong synergy between Hf-DBB^{F}-Ir@CpG(+) based in situ cancer vaccination and CBI. See FIGS. 37A-37C.

### Example 14

### Adaptive Immunity

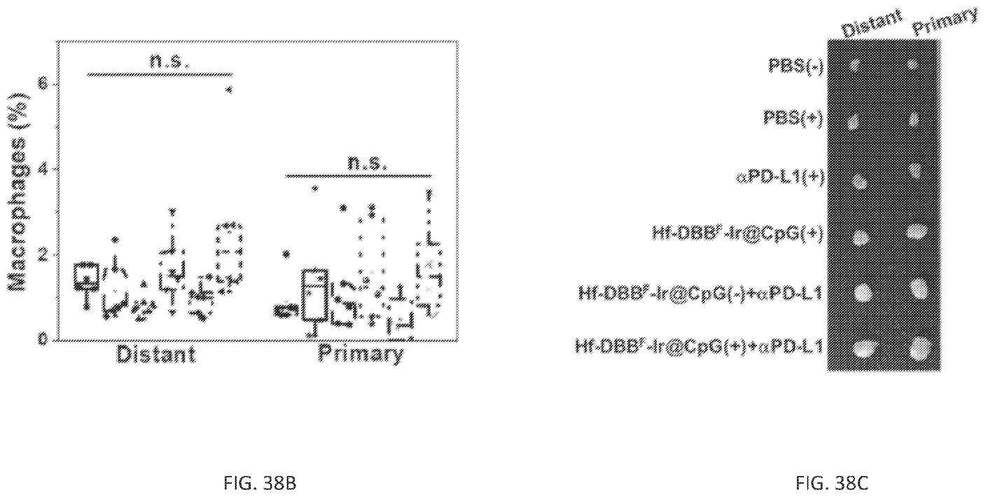

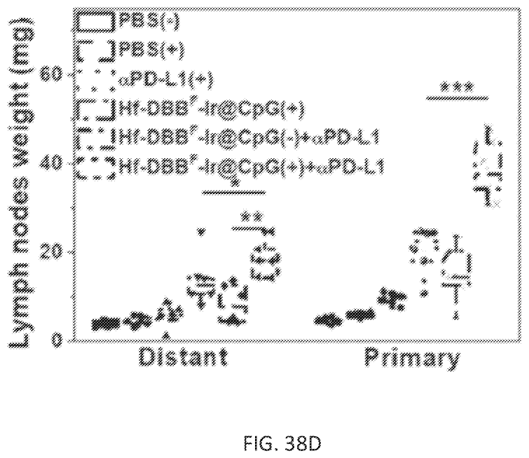

Infiltrating leukocytes were profiled in both primary and distant tumors 10 days post irradiation. Hf-DBB^{F}-Ir@CpG+αPD-L1(+) treatment group showed significant increase of tumor-infiltrating CD45⁺ leukocytes (see FIG. 37D), DCs (see FIG. 37E), macrophages (see FIGS. 38A and 38B), and CD8⁺ T cells (see FIG. 37F) in both primary and distant tumors, implying a strengthened innate immune response after in situ vaccination. Specifically, after treatment with Hf-DBB^{F}-Ir@CpG(+)+αPD-L1, the percentages of natural kill cells (NK cells, see FIG. 37G), CD4⁺ T cells (see FIG. 37H) and CD8⁺ T cells (see FIG. 37I) of the total primary and distant tumor cells significantly increased to 0.52±0.42% and 1.21±0.89%, 0.25±0.23% and 1.15±1.1400 and 1.46±0.59% and 1.43±0.55% from 0.06±0.05% and 0.13±0.19%, 0.05±0.04% and 0.03±0.02%, and 0.41±0.30% and 0.36±0.27% in PBS(−) group, respectively. The effector T cell infiltration was shown by both flow cytometry and CLSM. DLNs on both sides were harvested, weighed and immunostained for detecting T cell expansion, suggesting that Hf-DBB^{F}-Ir@CpG+αPD-L1(+) treatment promoted T cell expansion on bilateral DLNs. See FIGS. 38C and 38D. These results suggest that the combination of Hf-DBB^{F}-Ir@CpG(+)+αPD-L1 not only induces innate immune response but also augments adaptive immunity in both treated local and untreated distant tumors.

### Example 15

### Induced and Long-Term Immunity

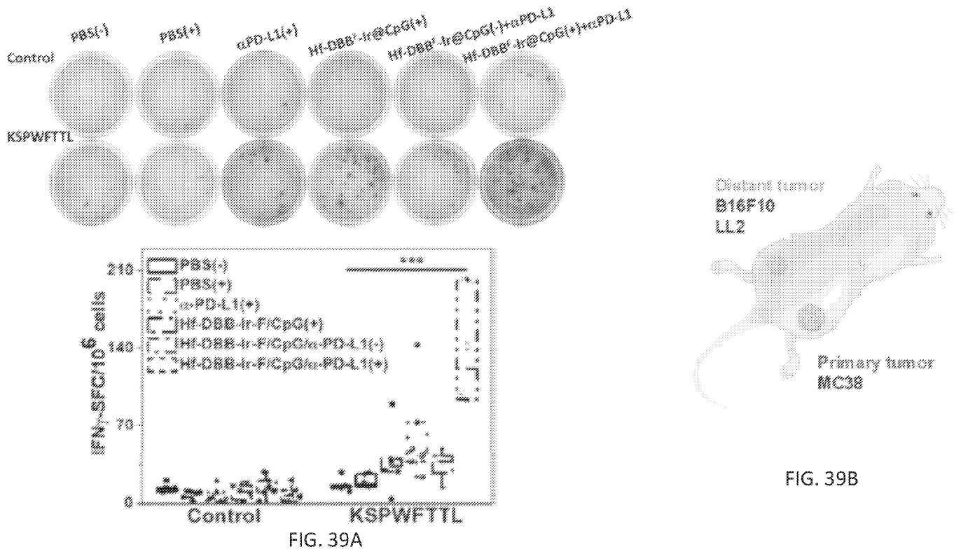

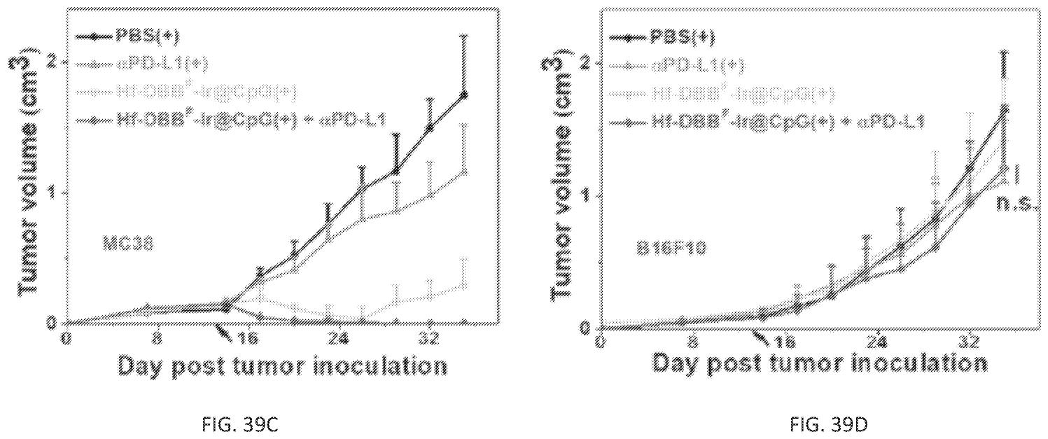

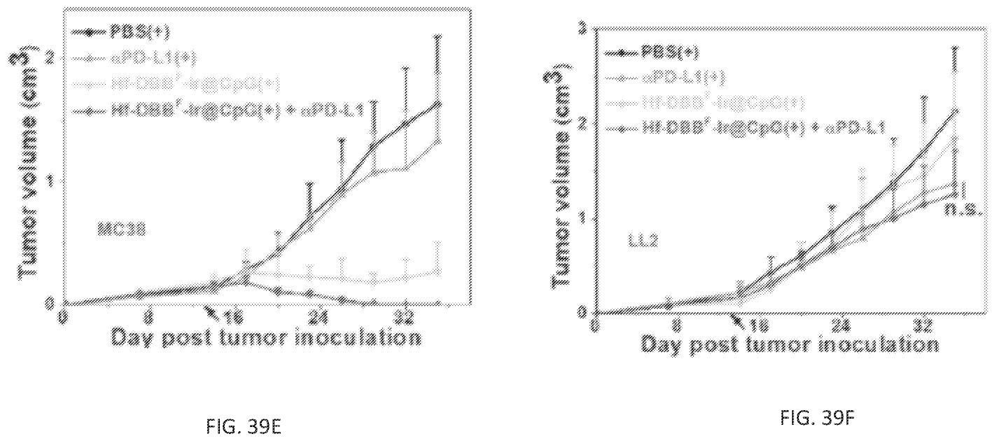

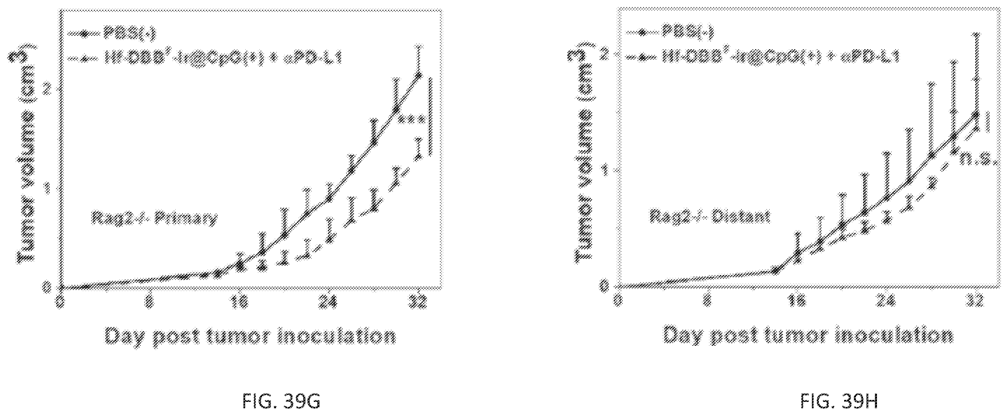

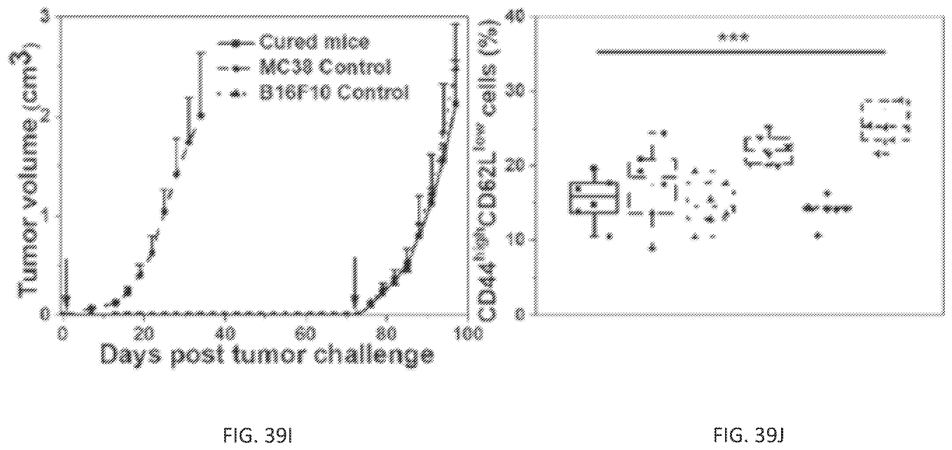

Specificity of induced immunity: The presence of tumor-antigen specific cytotoxic T cells was determined with an IFN-γ Enzyme-Linked ImmunoSpot (ELISpot) assay. Splenocytes were harvested from MC38-bearing mice 10 days post first irradiation and stimulated with the peptide sequence KSPWFTTL (SEQ ID NO: 5) for 42 hours. IFN-γ spot forming cells were counted with an Immunospot Reader. The number of antigen-specific IFN-γ producing T cells per 10⁶ splenocytes significantly increased in tumor-bearing mice treated with Hf-DBB^{F}-Ir@CpG(+) and Hf-DBB^{F}-Ir@CpG(+)+αPD-L1 (60.2±39.6 and 139.0±52.4 compared to 16.4±5.9 for PBS(−), see FIG. 39A), suggesting that both Hf-DBB^{F}-Ir@CpG(+) and Hf-DBB^{F}-Ir@CpG(+)+αPD-L1 effectively generate tumor-specific T cell responses. To further investigate the specific antitumor immunity, MC38 primary tumors were treated with Hf-DBB^{F}-Ir@CpG(+) or Hf-DBB^{F}-Ir@CpG(+)+αPD-L1 to observe if the treatment could regress unmatched syngeneic tumors on distant flanks. As illustrated in FIG. 39B, MC38 were used as the primary treated tumors and syngeneic tumor cell lines B16F10 and LL2 were implanted concurrently as the distant untreated tumors. Both Hf-DBB^{F}-Ir@CpG(+) and Hf-DBB^{F}_Ir@CpG(+)+αPD-L1 treatments effectively regressed the primary MC38 tumors but had no effect on the distant B16F10 or LL2 tumors. See FIG. 39C-39F. These experiments indicate tumor-specificity and personalized nature of the newly expanded T cells following in situ vaccination with the Hf-DBB^{F}-Ir@CpG(+)+αPD-L1 treatment.

Long-term antitumor immunity: The involvement of cytotoxic T cells in efficient abscopal effect was further supported by the lack of efficacy of Hf-DBB^{F}_Ir@CpG(+)+αPD-L1 treatment on a bilateral subcutaneous model of MC38 on Rag2^{-/-} C57BL/6 mice deficient of mature T and B cells. The primary tumors treated with Hf-DBB^{F}-Ir@CpG(+)+αPD-L1 were initially suppressed (see FIG. 39G), but grew rapidly after the end of X-ray irradiation. No abscopal effect was observed on the distant tumors. See FIG. 39H. This result confirms that both the abscopal effect and local tumor regression/eradication require the presence of tumor-specific adaptive immunity. Finally, a tumor rechallenge study was carried out to confirm the long-term immune memory effect. For the mice completely cured after treatment with Hf-DBB^{F}_Ir@CpG(+)+αPD-L1, 5×10⁵ MC38 cells were inoculated on the contralateral, left flank 30 days post tumor eradication and those cured mice remained tumor-free after first challenge, indicating strong antitumor immune memory effect. 2 months after the first challenge, 2×10⁶ B16F10 cells were inoculated on the right flank and the cured mice established tumors similarly to naïve mice, suggesting the tumor-specificity of the immune memory effect. See FIG. 39I. Memory effector cells (CD3ε⁺CD8α⁺CD44^{high}CD62L^{low} phenotype) were also profiled in splenocytes after the combination treatment. As shown in FIG. 39J, significant increase of memory effector cells was observed in spleens after Hf-DBB^{F}-Ir@CpG(+)+αPD-L1 treatment.

### Example 16

### Discussion of Examples 9-15

Advanced tumors escape immune surveillance by inactivating, dysregulating and hijacking host immune systems (Mahoney et al., 2015; Dunn et al., 2002). To combat this, anti-PD-(L)1 CBI has become a standard of care for some cancers by targeting T cell inhibitory checkpoint signaling pathways to afford durable anticancer efficacy with low side effects (Brahmer et al., 2012; Errico, 2015). Immune checkpoint inhibition, however, only elicits durable responses in a minority of cancer patients due to the reliance on immunogenic tumor microenvironments, so-called “hot” tumors. For patients with relatively “cold” tumors, e.g., low tumor mutation burden, low PD-L1 expression level and/or low abundance of pre-existing T cells, immunoadjuvant treatments to turn “cold” tumors “hot” are actively examined in combination with checkpoint inhibitors to overcome immune tolerance and potentiate antitumor immunity in the host system.

According to the presently disclosed subject matter, it is proposed that local treatment to generate innate immunity with tumor antigen exposure can effectively reinvigorate “cold” tumors to immunogenic hotbeds. Furthermore, two pattern recognition receptor (PRR) pathways (Kawai and Akira, 2010; Gong et al., 2019), cGAS-STING induced by DAMPs after RT damage (Deng et al., 2014) and TLR pathway induced by PAMPs like CpG (Weiner et al., 1997) operate independently (Emming and Schroder, 2019), suggesting that they can be activated simultaneously to achieve an additive or synergistic effect on immune stimulation. Porous nMOFs built from Hf-oxo SBUs and photosensitizing ligands can enhance radiotherapeutic effects of ionizing radiations with enhanced X-ray energy deposition, facile ROS diffusion, and unique RT-RDT mode of action (Lan et al., 2018; Ni et al., 2019; Lu et al., 2018). The presently disclosed subject matter provides new cationic Hf-based nMOF, Hf-DBB^{F}_Ir, for non-viral in situ vaccination by mediating efficient RT-RDT to generate immunogenic tumor antigens and DAMPs and to deliver anionic CpG as PAMPs. It is believed that the presently disclosed Hf-DBB^{F}-Ir@CpG(+) provides the first treatment with synergistic DAMPs and PAMPs packaged in the in situ cancer vaccine in local tumors while engaging lymphoid organs for antigen presentation to synergize with CBI to induce CTL infiltration in distant tumors. Furthermore, the 83.3% cure rate achieved by Hf-DBB^{F}-Ir@CpG(+)+αPD-L1 on a relatively immunosuppressive 14-day MC38 colorectal cancer model suggests the potential use of nMOF-based in situ vaccines on immunologically “cold” tumors.

The in situ cancer vaccination afforded by nMOFs has several potential advantages over traditional cancer vaccines. First, the in situ vaccine afforded by nMOFs is personalized from autologous antigens released from tumors by a myriad of ROSs, and can overcome the tumor heterogeneity issue facing traditionally manufactured peptide vaccines. Second, cationic nMOFs can capture DAMPs and tumor antigens from dying cancer cells via electrostatic interactions (Min et al., 2017), and with virus-like size distribution, can be recognized and taken up by APCs for efficient antigen presentation to stimulate a strong cytotoxic T-cell response. Third, cationic nMOFs deliver and protect anionic CpGs from enzymatic degradation for TLR stimulation and downstream immunologic processes. Fourth, tumor antigens and DAMPs released by the nMOF-mediated RT-RDT process and CpG-based PAMPs delivered by cationic nMOFs work synergistically to stimulate DC maturation to promote antigen presentation and adaptive immunity. Fifth, the nMOF-based vaccine is activated by X-rays to release DAMPs and tumor antigens with relatively nontoxic components and is thus expected to have few side effects. Furthermore, systemic administration of αPD-L1 blocks the immunosuppressive co-inhibitory marker PD-L1 to augment antigen presentation and attenuate T cell exhaustion. The combination of nMOF-mediated in situ cancer vaccine with CBI affords tumor-specific and long-term antitumor immunity.

In summary, the presently disclosed subject matter provides a novel nMOF by rationally fluorinating photosensitizing ligands for effective ROS generation through RT-RDT and tuning nMOF surface charge for efficient CpG loading. Following intratumoral administration of Hf-DBB^{F}-Ir and X-ray irradiation, the in situ released DAMPs and tumor antigens and CpGs delivered by Hf-DBB^{F}-Ir synergistically function as a potent personalized cancer vaccine to activate APCs and expand cytotoxic T cells in tumor-draining lymph nodes to reinvigorate the adaptive immune system for local tumor regression. When combined with an immune checkpoint inhibitor, innate and adaptive immunity from the nMOF-based cancer vaccine was further enhanced to generate superb antitumor efficacy with tumor specificity and long-term immune memory effect. This combination treatment extends the local therapeutic effects of the in situ cancer vaccine to distant tumors via systemic antitumor immunity by re-activating CTLs. This study paves the way to advance the concept of nMOF-based personalized vaccines into human trials for the treatment of advanced cancers.

### Example 17

### nMOFs and Peptides

Hf-DBP-Pt nMOF with acetate (OAc) capping groups was synthesized in a similar fashion as Hf-DBP. Similar to Hf-DBP, Hf-DBP-Pt were formed by connecting Hf₁₂ SBUs with DBP-Pt ligands in an hcp-like stacking pattern. The Hf₁₂ SBUs on the surface were also terminated by OAc groups with a ζ-potential of −22.5±0.5 mV in H₂O. Hf-DBP-Pt was treated with trimethylsilyl trifluoroacetate (TMS-TFA) to afford TFA-modified Hf-DBP-Pt by replacing the OAc groups with TFA groups as determined by ¹H and ¹⁹F NMR spectroscopy. TEM imaging and PXRD studies showed that the morphology and crystallinity of Hf-DBP-Pt were maintained after surface modification. The weekly coordinating TFA groups can be replaced by carboxylate groups in proteins or phosphate groups on nucleic acids.

MUC-1 peptide is a short peptide (d-CQCRRKN) (SEQ ID NO: 1) targeting membrane MUC-1 mucins (CQC motif), which can induce cell apoptosis and initiate host anti-cancer immune responses. Therapeutic peptides have faced great challenges like low cellular uptake and low stability in vivo. Membrane penetrating peptide GO-203 (RRRRRRRRRCQCRRKN) (SEQ ID NO: 2) was developed to target MUC-1 mucins but the clinical efficacy was unremarkable. By nMOF conjugation with MUC-1 peptide, it was found that Hf-DBP-Pt-TFA was not only able to deliver MUC-1 more efficiently in vitro, but MUC-1 peptide can also synergize with RT-RDT to perform better anti-cancer efficacy on MC38 bearing C57BL/6 subcutaneous model.

MUC-1/Hf-DBP-Pt was prepared by mixing 1 mM TFA modified Hf-DBP-Pt and 2 mM MUC-1 peptides in water solution. The suspension was vortexed every 5 minutes for a total of 15 minutes to afford MUC-1/Hf-DBP-Pt. TEM imaging and PXRD studies showed that the morphology and crystallinity were maintained after modulator exchange.

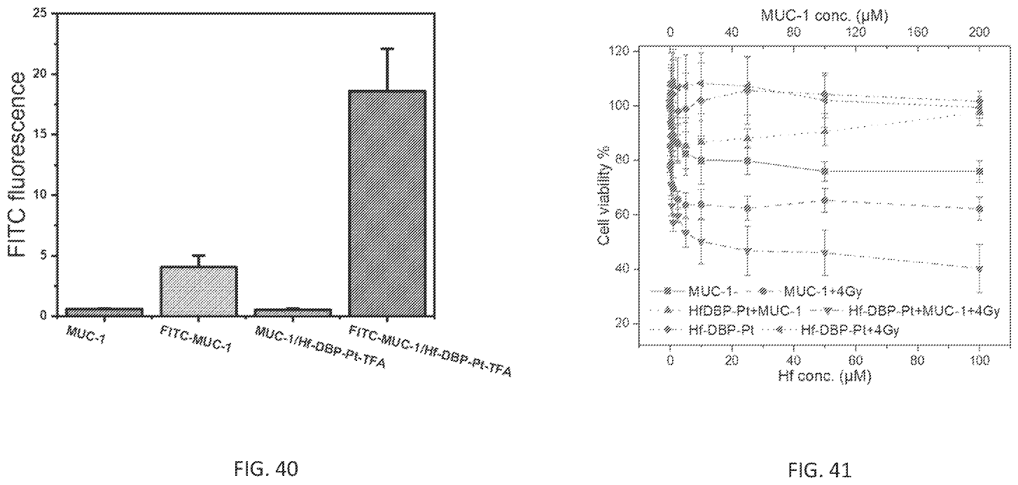

Cellular uptake: HEK293T cells were seeded in 6 well plates with coverslips at a density of 2×10⁵ cells/mL and cultured overnight. 10 μM TFA modified Hf-DBP-Pt and 20 μM FITC-MUC-1 were mixed in water, vortexed and the mixture was let to stand still for 15 minutes. Then 40 μL of the mixture was added to the 2 mL medium, and the control wells were added with same concentration of FITC-MUC-1 or MUC-1/Hf-DBP-Pt (without fluorescence labeling). After 4 hours, the cells were washed and fixed with 4% PFA and observed under confocal laser scanning microscope. Green channels showed FITC-MUC-1 was delivered much more efficiently by Hf-DBP-Pt. See FIG. 40.

Cytotoxicity: MC38 cells were seeded in 96 well plates at a density of 1500 cells/well and cultured overnight. 1000 μM TFA modified Hf-DBP-Pt and 2000 μM MUC-1 were mixed in water, vortexed and the mixture was let to stand still for 15 minutes. Then the mixture was added to each well with various concentrations. Three plates were taken for 4 Gy X ray 4 hours after drug addition. MTS assay was performed 3 days later, and MUC-1/Hf-DBP-Pt showed synergistic effects of MUC-1 and RT-RDT upon X ray irradiation. See FIG. 41.

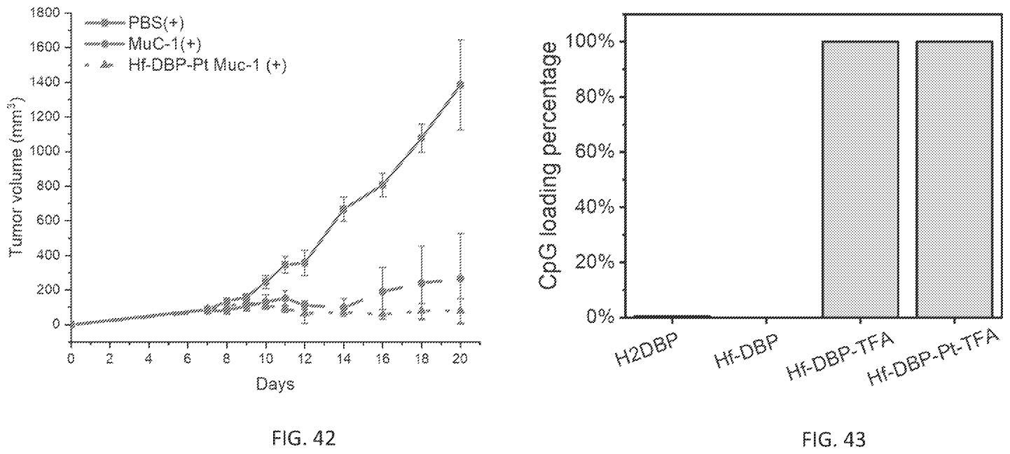

In vivo efficacy: 6-8-week-old C57BL/6 mice were inoculated with 2×10⁶ MC38 cells subcutaneously. MUC-1/Hf-DBP-Pt (0.4 μmol/0.2 μmol) was injected intratumorally when tumor reached ˜100 mm³. 8 hours later the mice were anesthetized and irradiated with 1 Gy X ray for the first time. The mice were then irradiated with 1 Gy X ray in the following 5 consecutive days as a total 6 Gy dose. The tumor volumes and body weights were measured daily. MUC-1/Hf-DBP-Pt showed synergistic therapeutic effects of RT-RDT and MUC-1. See FIG. 42. Steady body weight trend showed minimal toxicity and good biocompatibility of this system.

### Example 18

### nMOFs and CpG ODNs

CpG adsorption by different nMOFs: CpG ODN 2395 (3 μg) and different nMOFs (Hf-DBP, Hf-DBP-TFA, or Hf-DBP-Pt-TFA; 0.1 μmol) were mixed in water to prepare CpG/nMOF in separate 1.5 mL ep tubes. The mixture was let to stand still for 15 minutes and the mixture was centrifuged at 14500 rpm for 15 minutes. The DNA concentration of the supernatant was determined by NanoDrop. The CpG loading was then calculated for each kind of nMOFs. Hf-TBP and Hf-TBP-Pt can adsorb ˜80% of CpG and Hf-DBP-TFA, Hf-DBP-Pt, and Hf-DBP-Pt-TFA can adsorb >90% of CpG. See FIG. 43. However, Hf-DBP without TFA modification cannot adsorb CpG in this case.

### Example 19

### nMOFs and STING Agonist

Preparation of cGAMP/Hf-DBP-Pt: 0.2 μmol TFA-modified Hf-DBP-Pt and 5 g 2′,3′-cGAMP were mixed in a 30 μL water suspension, vortexed every 5 min for a total of 15 min.

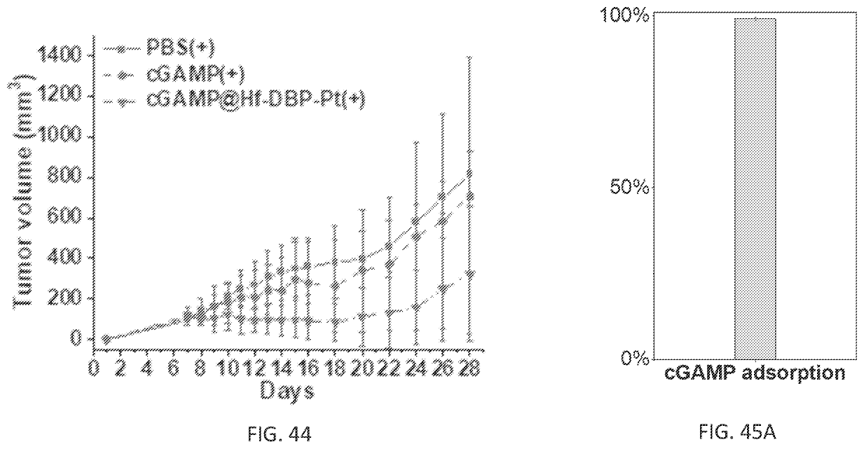

In vivo efficacy: 6-8-week-old C57BL/6 mice were inoculated with 2×10⁶ MC38 cells subcutaneously. PBS, cGAMP, or cGAMP/Hf-DBP-Pt (5 μg/0.2 μmol) was injected intratumorally when tumor reached ˜100 mm³ on Day 7. 8 hours later the mice were anesthetized and irradiated with 2 Gy X ray. The mice were further irradiated with 2 Gy X ray for 4 consecutive days with a total of 10 Gy X-ray dose. The tumor volumes and body weights were measured daily. As shown in FIG. 44, cGAMP/Hf-DBP-Pt showed synergistic therapeutic effects of RT-RDT and the STING agonist. Steady body weight trend showed minimal toxicity and good biocompatibility of this system.

Preparation of Hf₁₂-Ir nMOL: MOL is a subclass of MOF with a monolayered thickness. Hf₁₂-Ir nMOL was synthesized as follows: 500 μL of HfCl₄ solution [2.0 mg/mL in N,N-dimethylformamide (DMF)], 500 μL of H₂DBB-Ir-F solution (4.0 mg/mL in DMF), 2 μL of trifluoroacetic acid (TFA), and 5 μL of water were added to a 1-dram glass vial. The mixture was sonicated and heated in an 80° C. oven for 1 day. The yellow suspension was collected by centrifugation and washed with DMF and ethanol. The final product Hf₁₂-Ir nMOL was dispersed in ethanol for characterization and further use.

Hf₁₂-Ir nMOL contains Hf₁₂ secondary building units (SBUs) and Ir(DBB)[dF(CF₃)ppy]₂⁺ photosensitizing ligands. PXRD patterns verified Hf₁₂-Ir nMOL as a crystalline material. TEM and AFM revealed the morphology of Hf₁₂-Ir nMOL an ultrathin plate of <2 nm in thickness and around 100-200 nm in diameter.

Preparation of cGAMP nMOL: To prepare cGAMP/nMOL, the Hf₁₂-Ir nMOL was first dispersed in 100 μL nuclease-free water at an equivalent Hf concentration of 2 mM. 1 μg 2′3′-cGAMP was then added to the nMOL suspension. The mixture was vortexed every 5 minutes three times to afford the cGAMP/nMOL nanoconjugate. The concentrations of Hf were detected by inductively coupled plasma-mass spectrometry (ICP-MS) using an Agilent 7700×ICP-MS (Agilent Technologies, Santa Clara, California, United States of America) and analyzed using an ICP-MS MassHunter version B01.03 (Agilent Technologies, Santa Clara, California, United States of America). Samples were digested in concentrated HNO₃ (trace metal grade) with 1% HF acid solution for 2 days and then diluted in a final concentration of 2% HNO₃ matrix. The crystallinity of both nanoparticles were examined by powder X-ray diffraction (PXRD) on a Bruker D8 Venture diffractometer (Bruker, Billerica, Massachusetts, United States of America) using a Cu K_{α} radiation source (λ=1.54178 Å). The sizes and ζ-potentials were measured by a Malvern Nano Series ZetaSizer (Malvern Panalytical, Malvern, United Kingdom). The morphologies were observed by transmission electron microscopy (TEM) on a TECNAI Spirit TEM (FEI Company, Hillsboro, Oregon, United States of America) and atomic force microscopy (AFM) on a Bruker V/Multimode 8 instrument (Bruker, Billerica, United States of America). AFM, TEM imaging and PXRD studies showed that the morphology and crystallinity were maintained after cGAMP conjugation.

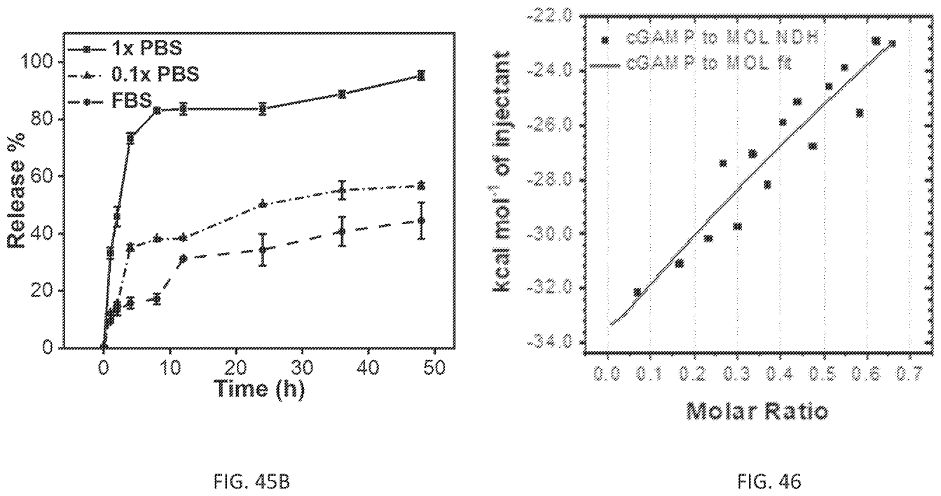

cGAMP loading efficiency and release profile of cGAMP/nMOL: The concentration of 2′3′-cGAMP was quantified by LC-MS on an Agilent 6540 Q-Tof MS-MS with 1290 UHPLC (5 m Agilent C18 reverse phase column) (Agilent Technologies, Santa Clara, California, United States of America). The standard curve of 2′3′-cGAMP was prepared by dissolving lyophilized 2′3′-cGAMP powder in nuclease-free water to afford 1000 ppm stock solution. The gradient dilutions were prepared, and the linear range was between 50 ppb and 20 ppm. The elution of the LC-MS was set as: 0-5 min, 95% H₂O 5% MeOH. The flow rate was 0.5 mL/min with injection volume of 20 μL. To determine the loading efficiency of 2′3′-cGAMP on Hf₁₂-Ir nMOL, GAMP/nMOL was freshly prepared as above and the supernatant was collected by centrifugation at 14000 g. The 2′3′-cGAMP concentration in the supernatant was quantified by LC-MS, N=3. For the release profile, cGAMP/nMOL was freshly prepared and redispersed in the same volume of 1×PBS, 0.1×PBS and FBS (100 μL/tube) in 1.5 mL Eppendorf tubes (3 replicates for each time point), respectively. The Eppendorf tubes were transferred onto a 37° C. heat block and the supernatants (80 L/tube) were collected at 0 h, 1 h, 2 h, 4 h, 8 h, 12 h, 24 h, 36 h, and 48 h by centrifugation at 14000 g. The supernatants in 1×PBS and 0.1 PBS groups were analyzed by LC-MS. The supernatants in the FBS group was mixed with 320 μL methanol per tube, sonicated for 1 minute to afford white precipitates, centrifuged again at 14000 g and the supernatants were then analyzed by LC-MS. As shown in FIG. 45A, TFA modified nMOL had nearly 100% adsorption efficiency, which verifies the conjugation between phosphate groups on cGAMP and Hf₁₂ SBUs on the nMOL. Fast release in 1×PBS was caused by phosphate exchange on SBUs. However, in 0.1×PBS and FBS which mimic the real phosphate concentration in biomedical application, the release of cGAMP was significantly slower. See FIG. 45B.

Isothermal titration calorimetry: The interaction between 2′3′-cGAMP and Hf₁₂-Ir nMOL was measured and analyzed on a MicroCal iTC₂₀₀ system (Malvern Instruments, Malvern, United Kingdom) equipped with reference and sample cells (V=L). All titrations were carried out using a 40 μL syringe at 298.15 K with a stirring rate of 250 rpm. 75 μM Hf₁₂-Ir nMOL water solution was titrated with 235 μM cGAMP water solution. Data analysis was performed using the MicroCal iTC₂₀₀ software (Malvern Instruments, Malvern, United Kingdom), and all data were fitted to an independent single-site model. The moderate binding interaction (K=3.80×10³ M⁻¹) verifies the dynamic binding between cGAMP and nMOL. See FIG. 46.

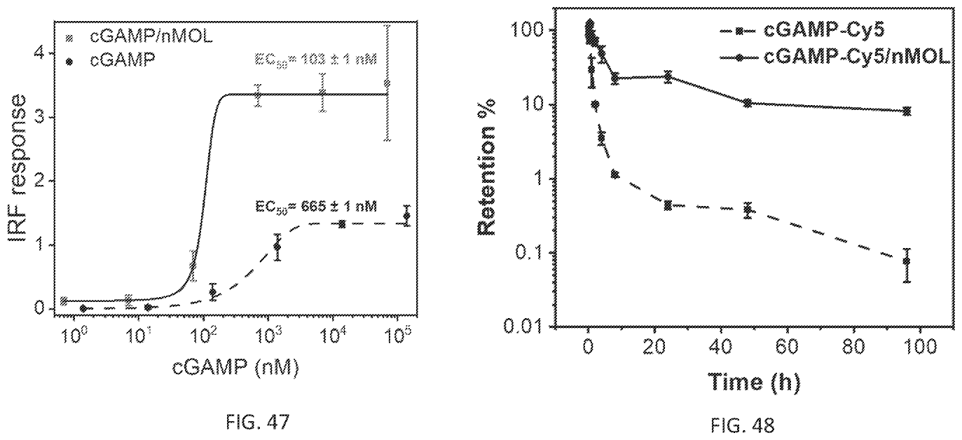

STING activation in vitro: Reporter cells sold under the tradename THP1-DUAL™ KO-MyD88 cells (InvivoGen, San Diego, California, United States of America) were used to quantify STING activation of free 2′3′-cGAMP and cGAMP/nMOL in vitro. The cells were seeded in 96-well plates at a density of 10⁵ cells/mL (N=6) and up to 10 μM 2′3′-cGAMP and cGAMP/nMOL were added and incubated for 24 hours. The stimulation of interferon regulatory factor (IRF) pathway was quantified by an assay sold under the tradename QUANTI-LUC™ (InvivoGen, San Diego, California, United States of America) on a plate reader sold under the tradename SYNERGY™ HTX (BioTek, Winooski, Vermont, United States of America) according to vendor's protocol. As shown in FIG. 47, cGAMP/nMOL had a much lower EC₅₀ (<1/6) to activate STING pathway in vitro, which shows the potential of cGAMP/nMOL as a promising nano-STING agonist.

In vivo imaging of cGAMP retention in tumors: Subcutaneous MC38 tumor bearing C57BL/6 mouse model was established as described in Example 17, above. When tumors reached ˜150 mm³, 20 μL of cGAMP-Cy5/nMOL (2 μg/0.5 μmol Hf) and cGAMP-Cy5 (2 μg) was intratumorally injected into the mice. The mice were anaesthetized with 2% (v/v) isoflurane/oxygen and imaged by an IVIS Spectrum 200 (Xenogen, Hopkinton, Massachusetts, United States of America; ex. 640 nm/em. 680 nm) at 10 minutes, 20 minutes, 30 minutes, 1 hour, 2 hours, 4 hours, 8 hours, 24 hours, 48 hours and 96 hours after injection. The images were processed and analyzed by software sold under the tradename LIVING IMAGE® 4.7.2 (PerkinElmer, Waltham, Massachusetts, United States of America). After 8 hours, the cGAMP-Cy5/nMOL had at least one magnitude higher fluorescence signal retention than free cGAMP. See FIG. 48. Free cGAMP diffused and disappeared quickly in vivo, but cGAMP/nMOL slowly released cGAMP for a longer STING activation.

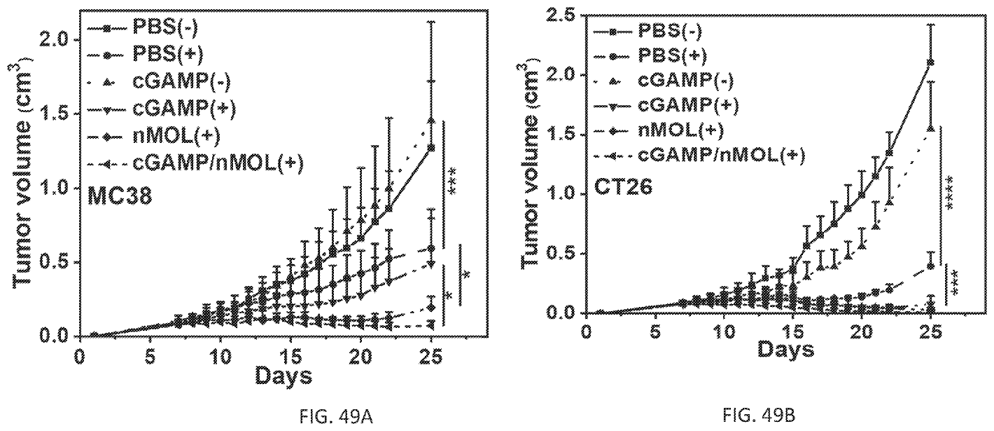

In vivo antitumor efficacy: The antitumor efficacy of cGAMP/nMOL was evaluated on subcutaneous CT26 tumor-bearing BALB/c and MC38 tumor-bearing C57BL/6 mouse models. For single tumor model, 2×10⁶ CT26 cells or MC38 cells were subcutaneously injected into the right flanks of BALB/c or C57BL/6 mice, respectively (both N=6). When the tumors reached 75-100 mm³ on Day 7, 20 μL of Hfiz-Ir nMOL (0.5 μmol Hf), cGAMP/nMOL (2 μg/0.5 μmol Hf), cGAMP (2 μg) or PBS was intratumorally injected into the mice. After 8 hours, the mice were anaesthetized with 2.5% (v/v) isoflurane/oxygen and the tumors were irradiated with 2 Gy X-ray/fraction for 6 consecutive days. The tumor volumes, body weights and health conditions of the mice were closely monitored. As shown in FIGS. 49A and 49B, synergistic effects between RT-RDT and STING activation achieved local tumor control at 1/5 of typical dose of cGAMP (10 μg).

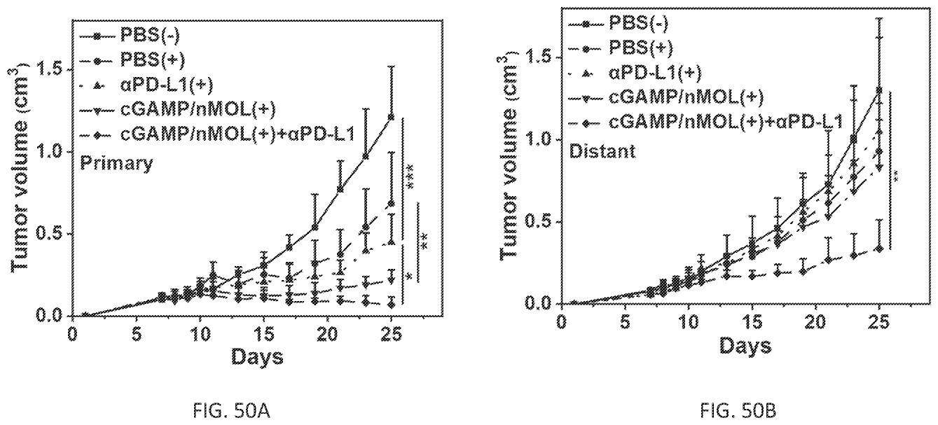

Abscopal effect: For bilateral MC38 tumor model, 2×10⁶ MC38 cells were subcutaneously injected into the right flank and 1×10⁶ MC38 cells were injected into left flank of C57BL/6 mice (N=6). When the primary tumors (right) reached 100-125 mm³ on Day 7, the primary tumors were injected with 20 μL of cGAMP/nMOL (2 g/0.5 μmol Hf) or PBS. The mice received same procedure of X-ray treatment as above for single tumor models. The mice in αPD-L1 or cGAMP/nMOL+αPD-L1 group were intraperitoneally injected with 2×75 μg/mouse αPD-L1 antibody on day 3 and day 6 after first X-ray treatment. Checkpoint blockade immunotherapy by αPD-L1 enhanced cGAMP/nMOL for better disease control of both local and distant sites. See FIGS. 50A and 50B. PD-L1 blockade extended the local synergy between RT-RDT and STING to systemic anti-cancer immune responses. The therapeutic combination of cGAMP/nMOL+αPD-L1 provided: 1) proliferation inhibition of cancer mass by augmented radiosensitization; 2) tumor antigen exposure by RT-RDT; 3) maturation and activation of APCs and T cells by STING agonists; 4) PD-1/PD-L1 checkpoint blockade by CBI. These four compartments were orchestrated and integrated into the 2D nanoplatform to finally realize favorable immune responses and therapeutic outcomes.

### REFERENCES

All references listed herein including but not limited to all patents, patent applications and publications thereof, scientific journal articles, and database entries are incorporated herein by reference in their entireties to the extent that they supplement, explain, provide a background for, or teach methodology, techniques, and/or compositions employed herein.
Allavena, P.; Sica, A.; Garlanda, C.; Mantovani, A., The Yin-Yang of tumor-associated macrophages in neoplastic progression and immune surveillance. Immunol. Rev. 2008, 222 (1), 155-161.
An, J.; Geib, S. J.; Rosi, N. L., Cation-triggered drug release from a porous zinc-adeninate metal-organic framework. J. Am. Chem. Soc. 2009, 131 (24), 8376-8377.
Brahmer, J. R., et al., Safety and activity of anti-PD-L1 antibody in patients with advanced cancer. New England Journal of Medicine 2012, 366, 2455-2465.
Brody, J. D., et al., In situ vaccination with a TLR9 agonist induces systemic lymphoma regression: a phase I/II study. Journal of clinical oncology 2010, 28, 4324.
Castano, A. P., Mroz, P., Hamblin, M. R., Photodynamic therapy and anti-tumour immunity. Nature Reviews Cancer 2006, 6, 535.
Chao, M. P.; Jaiswal, S.; Weissman-Tsukamoto, R.; Alizadeh, A. A.; Gentles, A. J.; Volkmer, J.; Weiskopf, K.; Willingham, S. B.; Raveh, T.; Park, C. Y., Calreticulin is the dominant pro-phagocytic signal on multiple human cancers and is counterbalanced by CD47. Sci. Transl. Med. 2010, 2(63), 63ra94-63ra94.
Chao, Y., et al., Combined local immunostimulatory radioisotope therapy and systemic immune checkpoint blockade imparts potent antitumour responses. Nature Biomedical Engineering 2018, 2, 611.
Chen, H.; Wang, G. D.; Chuang, Y.-J.; Zhen, Z.; Chen, X.; Biddinger, P.; Hao, Z.; Liu, F.; Shen, B.; Pan, Z., Nanoscintillator-mediated X-ray inducible photodynamic therapy for in vivo cancer treatment. Nano Lett. 2015, 15 (4), 2249-2256.
Chen, Q.; Wang, C.; Zhang, X.; Chen, G.; Hu, Q.; Li, H.; Wang, J.; Wen, D.; Zhang, Y.; Lu, Y., In situ sprayed bioresponsive immunotherapeutic gel for post-surgical cancer treatment. Nat. Nanotechnol. 2019a, 14 (1), 89-97.
Chen, Q.; Chen, G.; Chen, J.; Shen, J.; Zhang, X.; Wang, J.; Chan, A.; Gu, Z., Bioresponsive Protein Complex of aPDI and aCD47 Antibodies for Enhanced Immunotherapy. Nano Lett. 2019b, 19 (8), 4879-4889.
Deng, L., et al., STING-dependent cytosolic DNA sensing promotes radiation-induced type I interferon-dependent antitumor immunity in immunogenic tumors. Immunity 2014, 41, 843-852.
Du, B., Yu, M., Zheng, J., Transport and interactions of nanoparticles in the kidneys. Nature Reviews Materials 2018, 1.
Dunn, G. P., Bruce, A. T., Ikeda, H., Old, L. J., R. D. Schreiber, R. D., Cancer immunoediting: from immunosurveillance to tumor escape. Nature immunology 2002, 3, 991-998.
Emming, S., Schroder, K., Tiered DNA sensors for escalating responses. Science 2019, 365, 1375-1376.
Errico, A., Immunotherapy: PD-1-PD-L1 axis: efficient checkpoint blockade against cancer. Nature reviews Clinical oncology 2015, 12, 63-63.
Feng, M.; Jiang, W.; Kim, B. Y.; Zhang, C. C.; Fu, Y.-X.; Weissman, I. L., Phagocytosis checkpoints as new targets for cancer immunotherapy. Nat. Rev. Cancer 2019, 19 (10), 568-586.
Figdor, C. G., de Vries, I. J. M., Lesterhuis, W. J., Melief, C. J., Dendritic cell immunotherapy: mapping the way. Nature medicine 2004, 10, 475.
Furukawa, H.; Cordova, K. E.; O'Keeffe, M.; Yaghi, O. M., The chemistry and applications of metal-organic frameworks. Science 2013, 341 (6149), 1230444.
Gilliet, M., Cao, W., Liu, Y.-J., Plasmacytoid dendritic cells: sensing nucleic acids in viral infection and autoimmune diseases. Nature Reviews Immunology 2008, 8, 594.
Goldman, B.; L. DeFrancesco, L., The cancer vaccine roller coaster. Nature biotechnology 2009, 27, 129.
Gong, T., Liu, L., Jiang, W., Zhou, R., DAMP-sensing receptors in sterile inflammation and inflammatory diseases. Nature Reviews Immunology, 2019, 1-18.
Hu, Z., Ott, P. A., Wu, C. J., Towards personalized, tumour-specific, therapeutic vaccines for cancer. Nature Reviews Immunology 2018, 18, 168.
Jaiswal, S.; Jamieson, C. H.; Pang, W. W.; Park, C. Y.; Chao, M. P.; Majeti, R.; Traver, D.; van Rooijen, N.; Weissman, I. L., CD47 is upregulated on circulating hematopoietic stem cells and leukemia cells to avoid phagocytosis. Cell 2009, 138 (2), 271-285.
Jiang, W., et al., Designing nanomedicine for immuno-oncology. Nature Biomedical Engineering 2017, 1, 0029.
Joyce, J. A., Fearon, D. T., T cell exclusion, immune privilege, and the tumor microenvironment. Science 2015, 348, 74-80.
Kamath, P.; Darwin, E.; Arora, H.; Nouri, K., A Review on Imiquimod Therapy and Discussion on Optimal Management of Basal Cell Carcinomas. Clin. Drug Investig. 2018, 38 (10), 883-899.
Kawai, T., S. Akira, S., The role of pattern-recognition receptors in innate immunity: update on Toll-like receptors. Nature immunology 2010, 11, 373.
Kepp, O., Marabelle, A., Zitvogel, L., Kroemer, G., Oncolysis without viruses-inducing systemic anticancer immune responses with local therapies. Nature Reviews Clinical Oncology 2019, 1-16.
Klinman, D. M., Immunotherapeutic uses of CpG oligodeoxynucleotides. Nature Reviews Immunology 2004, 4, 249.
Kojima, Y.; Volkmer, J.-P.; McKenna, K.; Civelek, M.; Lusis, A. J.; Miller, C. L.; Direnzo, D.; Nanda, V.; Ye, J.; Connolly, A. J., CD47-blocking antibodies restore phagocytosis and prevent atherosclerosis. Nature 2016, 536 (7614), 86-90.
Kuai, R., Ochyl, L. J., Bahjat, K. S., Schwendeman, A., Moon, J. J., Designer vaccine nanodiscs for personalized cancer immunotherapy. Nature materials 2017, 16, 489.
Lan, G., Ni, K., Veroneau, S. S., Song, Y., Lin, W., Nanoscale Metal-Organic Layers for Radiotherapy-Radiodynamic Therapy. Journal of the American Chemical Society 2018, 140, 16971-16975.
Lan, G., Ni, K., Lin, W., Nanoscale metal-organic frameworks for phototherapy of cancer. Coordination chemistry reviews 2019a, 379, 65-81.
Lan, G., et al., Nanoscale Metal-Organic Framework Hierarchically Combines High-Z Components for Multifarious Radio-Enhancement. J. Am. Chem. Soc. 2019b, 141, 6859-6863.
Liu, H., et al., Structure-based programming of lymph-node targeting in molecular vaccines. Nature 2014, 507, 519.
Liu, X.; Pu, Y.; Cron, K.; Deng, L.; Kline, J.; Frazier, W. A.; Xu, H.; Peng, H.; Fu, Y.-X.; Xu, M. M., CD47 blockade triggers T cell-mediated destruction of immunogenic tumors. Nat. Med. 2015, 21 (10), 1209.
Lou, J.; Zhang, L.; Zheng, G., Advancing cancer immunotherapies with nanotechnology. Adv. Ther. 2019, 2 (4), 1800128.
Louttit, C.; Park, K. S.; Moon, J. J., Bioinspired nucleic acid structures for immune modulation. Biomater. 2019, 119287.
Lu, K.; He, C.; Lin, W., Nanoscale Metal-Organic Framework for Highly Effective Photodynamic Therapy of Resistant Head and Neck Cancer. J. Am. Chem. Soc. 2014, 136 (48), 16712-16715.
Lu, K.; He, C.; Guo, N.; Chan, C.; Ni, K.; Lan, G.; Tang, H.; Pelizzari, C.; Fu, Y.-X.; Spiotto, M. T., Low-dose X-ray radiotherapy-radiodynamic therapy via nanoscale metal-organic frameworks enhances checkpoint blockade immunotherapy. Nat. Biomed. Eng. 2018, 2 (8), 600.
Luo, M., et al., A STING-activating nanovaccine for cancer immunotherapy. Nature nanotechnology 2017, 12, 648.
Lutz, M. B., Schuler, G., Immature, semi-mature and fully mature dendritic cells: which signals induce tolerance or immunity? Trends in immunology 2002, 23, 445-449.
Ma, L., et al., Enhanced CAR-T cell activity against solid tumors by vaccine boosting through the chimeric receptor. Science 2019, 365, 162-168.
Mahoney, K. M., Rennert, P. D., Freeman, G. J., Combination cancer immunotherapy and new immunomodulatory targets. Nature reviews Drug discovery 2015, 14, 561-584.
Min, Y., et al., Antigen-capturing nanoparticles improve the abscopal effect and cancer immunotherapy. Nature nanotechnology 2017, 12, 877.
Morris, W.; Briley, W. E.; Auyeung, E.; Cabezas, M. D.; Mirkin, C. A., Nucleic Acid-Metal Organic Framework (MOF) Nanoparticle Conjugates. J. Am. Chem. Soc. 2014, 136 (20), 7261-7264.
Nam, J.; Son, S.; Park, K. S.; Zou, W.; Shea, L. D.; Moon, J. J., Cancer nanomedicine for combination cancer immunotherapy. Nat. Rev. Mater. 2019, 4 (6), 398-414.
Ni, K., et al., Nanoscale metal-organic frameworks enhance radiotherapy to potentiate checkpoint blockade immunotherapy. Nature communications 2018a, 9, 2351.
Ni, K., et al., Nanoscale metal-organic frameworks for mitochondria-targeted radiotherapy radiodynamic therapy. Nature communications 2018b, 9, 4321.
Ni, K.; Lan, G.; Chan, C.; Duan, X.; Guo, N.; Veroneau, S. S.; Weichselbaum, R. R.; Lin, W., Ultrathin Metal-Organic-Layer Mediated Radiotherapy-Radiodynamic Therapy. Matter 2019, 1 (5), 1331-1353.
O'neill, L. A.; Golenbock, D.; Bowie, A. G., The history of Toll-like receptors-redefining innate immunity. Nat. Rev. Immunol. 2013, 13 (6), 453-460.
Purcell, A. W., McCluskey, J., Rossjohn, J., More than one reason to rethink the use of peptides in vaccine design. Nature reviews Drug discovery 2007, 6, 404.
Radovic-Moreno, A. F., et al., Immunomodulatory spherical nucleic acids. Proceedings of the National Academy of Sciences, U.S.A. 2015, 112, 3892-3897.
Rodell, C. B.; Arlauckas, S. P.; Cuccarese, M. F.; Garris, C. S.; Li, R.; Ahmed, M. S.; Kohler, R. H.; Pittet, M. J.; Weissleder, R., TLR7/8-agonist-loaded nanoparticles promote the polarization of tumour-associated macrophages to enhance cancer immunotherapy. Nat. Biomed. Eng. 2018, 2 (8), 578.
Rosi, N. L., et al., Oligonucleotide-modified gold nanoparticles for intracellular gene regulation. Science 2006, 312, 1027-1030.
Sahin, U., Türeci, Ö., Personalized vaccines for cancer immunotherapy. Science 2018, 359, 1355-1360.
Scheetz, L., et al., Engineering patient-specific cancer immunotherapies. Nature biomedical engineering 2019, 3, 768-782.
Shae, D., et al., Endosomolytic polymersomes increase the activity of cyclic dinucleotide STING agonists to enhance cancer immunotherapy. Nature nanotechnology 2019, 14, 269.
Song, W., Musetti, S. N., Huang, L., Nanomaterials for cancer immunotherapy. Biomaterials 2017, 148, 16-30.
Tanyi, J. L., et al., Personalized cancer vaccine effectively mobilizes antitumor T cell immunity in ovarian cancer. Science translational medicine 2018, 10, eaao5931.
Wang, S., et al., General and direct method for preparing oligonucleotide-functionalized metal-organic framework Nanoparticles. Journal of the American Chemical Society 2017, 139, 9827-9830.
Wang, H., Mooney D. J., Biomaterial-assisted targeted modulation of immune cells in cancer treatment. Nature Materials 2018, 17, 761-772.
Weichselbaum, R. R., Liang, H., Deng, L., Fu, Y.-X., Radiotherapy and immunotherapy: a beneficial liaison? Nature reviews Clinical oncology 2017, 14, 365.
Weiner, G. J., Liu, H.-M., Wooldridge, J. E., Dahle, C. E., Krieg, A. M., Immunostimulatory oligodeoxynucleotides containing the CpG motif are effective as immune adjuvants in tumor antigen immunization. Proceedings of the National Academy of Sciences 1997, 94, 10833-10837.
Wilson, D. S., et al., Antigens reversibly conjugated to a polymeric glyco-adjuvant induce protective humoral and cellular immunity. Nature materials 2019, 18, 175.
Wu, M. X.; Yang, Y. W., Metal-organic framework (MOF)-based drug/cargo delivery and cancer therapy. Adv. Mater. 2017, 29 (23), 1606134.
Xiong, Z.; Ohlfest, J. R., Topical Imiquimod has Therapeutic and Immunomodulatory Effects Against Intracranial Tumors. J. Immunother. 2011, 34 (3).
Zhang, Y.-N., et al., Nanoparticle size influences antigen retention and presentation in lymph node follicles for humoral immunity. Nano letters 2019, 19, 7226-7235.
Zhu, Y.-Y., et al., Merging Photoredox and Organometallic Catalysts in a Metal-Organic Framework Significantly Boosts Photocatalytic Activities. Angew. Chem. 2018, 130, 14286-14290.

It will be understood that various details of the presently disclosed subject matter may be changed without departing from the scope of the presently disclosed subject matter. Furthermore, the foregoing description is for the purpose of illustration only, and not for the purpose of limitation.

1. A metal-organic framework (MOF) having a surface modified to coordinatively or electrostatically bind to one or more therapeutic agents of interest, wherein said MOF comprises:
(a) a plurality of metal oxo cluster secondary building units (SBUs), wherein each of said metal oxo cluster SBUs comprises one or more first metal ions and one or more anions, wherein each of said one or more anions is coordinated to one or more of the one or more first metal ions; and
(b) a plurality of organic bridging ligands linking together the plurality of SBUs to form a two- or three-dimensional matrix;
wherein (i) a plurality of SBUs at a surface of the MOF each comprise a weakly coordinating anion as a SBU capping group anion or (ii) the plurality of organic bridging ligands comprise an organic bridging ligand comprising an electron-withdrawing group or ligand, a positive charge, or a combination thereof, optionally wherein the plurality of organic bridging ligands comprise a ligand comprising a nitrogen donor group coordinatively bound to a second metal ion, wherein said second metal ion is further coordinated to at least one second metal ligand comprising one or more electron-withdrawing groups; wherein a surface of said MOF has enhanced ability to coordinatively or electrostatically bind to one or more therapeutic agents of interest.

2. The MOF of claim 1, wherein said one or more first metal ions comprise at least one ion of a metal that absorbs ionizing radiation, optionally X-rays, and/or wherein said metal is selected from the group consisting of Hf, a lanthanide metal, Ba, Ta, W, Re, Os, Ir, Pt, Au, Pb, and Bi; further optionally wherein the first metal ion is a Hf ion.

3. The MOF of claim 1, wherein a plurality of SBUs at a surface of the MOF each comprise a weakly coordinating anion as a capping group, optionally wherein said weakly coordinating anion is selected from the group consisting of trifluoroacetate and triflate.

4. The MOF of claim 3, wherein the plurality of organic bridging ligands comprise a porphyrin substituted by at least two carboxylate groups, optionally wherein the plurality of organic bridging ligands comprise 5,15-di(p-benzoato)porphyrin (DBP).

5. The MOF of claim 3, wherein the MOF further comprises a small molecule therapeutic agent sequestered in pores and/or cavities of the two- or three-dimensional network, optionally wherein said small molecule therapeutic agent is a chemotherapeutic agent, a small molecule inhibitor and/or a small molecule immunomodulator.

6. The MOF of claim 5, wherein the MOF comprises a chemotherapeutic agent sequestered in pores and/or cavities of the two- or three-dimensional network, optionally wherein said chemotherapeutic agent is selected from cisplatin, carboplatin, paclitaxel, SN-35, and etoposide.

7. The MOF of claim 5, wherein the MOF comprises a small molecule inhibitor sequestered in pores and/or cavities of the two- or three-dimensional network, optionally wherein said small molecule inhibitor is selected from the group consisting of a PLK1 inhibitor, a Wnt inhibitor, a Bcl-2 inhibitor, a PD-L1 inhibitor, an ENPP1 inhibitor and an IDO inhibitor.

8. The MOF of claim 5, wherein the MOF comprises a small molecule immunomodulator sequestered in pores and/or cavities of the two- or three-dimensional network.

9. The MOF of claim 8, wherein the small molecule immunomodulator is imiquimod (IMD).

10. The MOF of claim 1, wherein the plurality of organic bridging ligands comprise an organic bridging ligand comprising a nitrogen donor group, wherein said nitrogen donor group is coordinated to a second metal ion and wherein said second metal ion is further coordinated to at least one second metal ligand comprising one or more electron-withdrawing groups, optionally wherein the one or more electron withdrawing groups are selected from halo and perhaloalkyl groups.

11. The MOF of claim 10, wherein the organic bridging ligand comprising a nitrogen donor group is 4,4′-di(p-benzoato)-2,2′-bipyridine (DBB).

12. The MOF of claim 10, wherein the second metal ion is an iridium (Ir) ion or a ruthenium (Ru) ion and/or wherein said second metal ion is coordinated to two second metal ligands, wherein one or both of the second metal ligands comprise one or more electron withdrawing groups.

13. The MOF of claim 12, wherein one or both of the second metal ligands is 2-(2,4-difluorophenyl)-5-(trifluomethyl)pyridine (dF(CF₃)ppy).

14. The MOF claim 10, wherein the MOF has a zeta (Q)-potential value of at least about 5 millivolts (mV), optionally wherein the MOF has a C-potential value of at least about 30 mV.

15. The MOF of claim 1, wherein said MOF comprises a three-dimensional network, wherein said three-dimensional network is provided in the form of a nanoparticle.

16. A metal-organic framework (MOF) for the delivery of one or more therapeutic agents of interest, wherein said MOF comprises:
(a) a plurality of metal oxo cluster secondary building units (SBUs), wherein each of said metal oxo cluster SBUs comprises one or more first metal ions and one or more anions, wherein each of said anions is coordinated to one or more of the one or more first metal ions;
(b) a plurality of organic bridging ligands linking together the plurality of SBUs to form a two- or three-dimensional matrix; and
(c) one or more therapeutic agents of interest bonded to a surface of said MOF via coordinative bonds or electrostatic interactions, optionally wherein one or more therapeutic agents of interest are coordinatively bonded to a metal ion of one or more of the plurality of SBUs at the surface of the MOF.

17. The MOF of claim 16, wherein said first metal ion is an ion of a metal that absorbs ionizing radiation, optionally X-rays, and/or wherein the first metal ion is an ion of a metal selected from Hf, a lanthanide metal, Ba, Ta, W, Re, Os, Ir, Pt, Au, Pb, and Bi; further optionally wherein the first metal ion is a Hf ion.

18. The MOF of claim 16, wherein each of said one or more therapeutic agents of interest are selected from the group consisting of a nucleic acid, a small molecule comprising a phosphate or carboxylate group, and/or a macromolecule comprising a surface accessible phosphate or carboxylate group.

19. The MOF of claim 18, wherein the one or more therapeutic agents of interest comprise a macromolecule comprising a surface accessible phosphate or carboxylate group and wherein said macromolecule is a protein, optionally wherein said protein is an antibody.

20. The MOF of claim 19, wherein said protein is selected from the group consisting of an anti-CD37 antibody, an anti-CD44 antibody, an anti-CD47 antibody, an anti-CD73 antibody, an anti-PD-1 antibody, an anti-PD-L1 antibody, an anti-LAG3 antibody, and an anti-CTLA-4 antibody.

21. The MOF of claim 18, wherein the one or more therapeutic agents of interest comprise a nucleic acid and wherein said nucleic acid is selected from the group consisting of a miRNA, a mRNA, a siRNA, a CpG ODN, and a cyclic di-nucleotide, optionally wherein the nucleic acid is a cyclic di-nucleotide and said cyclic di-nucleotide is a STING agonist, further optionally wherein said STING agonist is c-di-AMP or cGAMP.

22. The MOF of claim 16, wherein said MOF comprises one or more additional therapeutic agents sequestered in pores or cavities of the two- or three-dimensional network; optionally wherein said MOF comprises about 1 wt % to about 50 wt % of said one or more additional therapeutic agents.

23. The MOF of claim 16, wherein the plurality of SBUs comprise Hf oxo clusters, wherein said plurality of organic bridging ligands comprise DBP, and wherein the one or more therapeutic agents of interest are bonded to the surface of said MOF via coordinative bonds to Hf ions of surface accessible SBUs.

24. The MOF of claim 23, wherein the one or more therapeutic agents of interest comprise one or more antibodies.

25. The MOF of claim 24, wherein the one or more therapeutic agents comprise an anti-CD47 antibody.

26. The MOF of claim 25, wherein the MOF further comprises IMD sequestered in pores or cavities of the two- or three-dimensional network.

27. The MOF of claim 26, wherein said MOF is a three-dimensional network and is provided as a nanoparticle.

28. The MOF of claim 27, wherein said MOF comprises about 1 wt % to about 50 wt % of the IMD or the anti-CD47 antibody; optionally wherein the MOF comprises about 9 weight (wt) % IMD and about 7.5 wt % anti-CD47 antibody.

29. The MOF of claim 16, wherein the plurality of SBUs comprise Hf oxo clusters, wherein said plurality of organic bridging ligands comprise DBB coordinated to an Ir ion, wherein said Ir ion is further coordinated to two (dF(CF₃)ppy); and wherein the one or more therapeutic agents of interest are bonded to the surface of said MOF via electrostatic interactions.

30. The MOF of claim 29, wherein the one or more therapeutic agents of interest comprise a nucleic acid.

31. The MOF of claim 30, wherein the nucleic acid is a STING agonist or a CpG oligodeoxynucleotide (ODN), optionally wherein the nucleic acid is a CpG ODN.

32. The MOF of claim 16, wherein the MOF comprises about 1 wt % to about 50 wt % of the one or more therapeutic agents of interest, optionally wherein said one or more therapeutic agents of interest comprise an antibody.

33. A method of treating cancer in a subject in need thereof, the method comprising:
(a) administering to the subject a MOF of claim 16; and
(b) exposing at least a portion of the subject to ionizing radiation energy, optionally X-rays.

34. The method of claim 33, wherein the method further comprises administering to said subject an additional therapeutic agent or treatment, optionally an immunotherapy agent and/or a cancer treatment selected from the group consisting of surgery, chemotherapy, toxin therapy, cryotherapy, and gene therapy.

35. The method of claim 34, wherein the additional therapeutic agent is an immunotherapy agent, optionally wherein said immunotherapy agent is an immune checkpoint inhibitor.

36. The method of claim 35, wherein the immunotherapy agent is an anti-PD-1 or an anti-PD-L1 antibody.

37. The method of claim 33, wherein the cancer is colorectal cancer, melanoma, head and neck cancer, brain cancer, breast cancer, liver cancer, cervical cancer, lung cancer or pancreatic cancer.

38. The method of claim 33, wherein administration of the MOF provides an extended release profile for one or more of the one or more therapeutic agents of interest, optionally wherein the release rate is tunable and/or wherein the MOF provides sustained release of one or more therapeutic agents of interest over a period of a few hours or a few days.

39. The method of claim 33, wherein administration of the MOF lowers the therapeutically effective dose of the one or more therapeutic agents of interest.

40. A method of enhancing surface interaction and/or bonding of one or more therapeutic agents of interest to a metal-organic framework (MOF), the method comprising modifying the surface of the MOF by (i) providing one or more surface accessible coordination sites coordinatively bonded to a weakly coordinated anion that can be replaced by a carboxylate or phosphate substituent of a therapeutic agent of interest or (ii) providing a MOF comprising one or more electron-withdrawing bridging ligands, one or more bridging ligands comprising a positive charge, or a combination thereof.

41. The method of claim 40, wherein modifying the surface of the MOF comprises:
(ia) providing a parent MOF comprising metal oxo cluster SBUs linked together via organic bridging ligands, wherein each of said SBUs comprises one or more metal ions and one or more anions, and wherein said MOF comprises a plurality of surface accessible metal oxo cluster SBUs where the one or more anions of each of said surface accessible metal oxo cluster SBUs comprise a strongly coordinating anion as a SBU capping group; optionally wherein said strongly coordinating anion comprises acetate or formate; and
(ib) removing said strongly coordinating anion, wherein the removing comprises contacting said parent MOF with a reagent selected from trimethylsilyl trifluoroacetate, trimethylsilyl triflate, and a mineral acid having a pKa of less than about 3; thereby replacing said strongly coordinating anion, optionally wherein said strongly coordinating anion is selected from an acetate or a formate anion, with a weakly coordinating anion, optionally wherein said weakly coordinating anion is selected from a trifluoroacetate or triflate anion.

42. The method of claim 40, wherein providing a MOF comprising one or more bridging ligands comprising an electron-withdrawing group, one or more bridging ligands comprising a positive charge, or a combination thereof comprises providing a MOF comprising metal oxo cluster SBUs linked together via organic bridging ligands, wherein each of said SBUs comprise one or more first metal ions and one or more anions coordinated to said one or more first metal ions, and wherein said organic bridging ligands comprise at least one organic bridging ligand comprising a coordinated, non-SBU-associated second metal ion, wherein said second metal ion is further coordinated to one or more electron-withdrawing ligand, optionally wherein said electron-withdrawing ligand is a halo and/or perhaloalkyl-substituted bipyridine ligand.

43. The method of claim 42, wherein providing the MOF comprises providing an MOF comprising a di(4-benzoato)-2,2′-bypyridine (DBB) bridging ligand, wherein said DBB bridging ligand is coordinated to a first metal ion of two different metal oxo cluster SBUs and to a second metal ion and wherein said second metal ion is further coordinated to two halo and/or perhaloalkyl-substituted pyridine ligands, optionally wherein said two halo and/or perhaloalkyl-substituted pyridine ligands are each 2-(2,4-difluorophenyl)-5-(trifluoromethyl)-pyridine.

44. The method of claim 42, wherein said second metal ion is iridium (Ir) or ruthenium (Ru).

45. The method of claim 40, wherein the MOF comprises one or more SBU comprising a metal ion that absorbs ionizing radiation, optionally x-rays and/or wherein the metal ion is an ion of an element selected from the group consisting of Hf, a lanthanide metal, Ba, Ta, W, Re, Os, Ir, Pt, Au, Pb, and Bi; further optionally wherein said metal ion is a Hf ion.

46. The method of claim 40, wherein said MOF has enhanced interaction and/or bonding ability for one or more therapeutic agents of interest compared to a MOF without surface modification, wherein said one or more therapeutic agents of interest are selected from a nucleic acid, a small molecule, and/or macromolecule comprising a surface accessible phosphate or carboxylate group.

47. The method of claim 46, wherein said protein is selected from the group consisting of an anti-CD37 antibody, an anti-CD44 antibody, an anti-CD47 antibody, an anti-CD73 antibody, an anti-PD-1 antibody, an anti-PD-L1 antibody, an anti-LAG3 antibody, and an anti-CTLA-4 antibody.

48. The method of claim 46, wherein said nucleic acid is selected from the group consisting of a miRNA, a mRNA, a siRNA, a CpG ODN, and a cyclic di-nucleotide, optionally wherein said cyclic di-nucleotide is a STING agonist, further optionally wherein said STING agonist is c-di-AMP or cGAMP.


SEQUENCE LISTING (5 sequences)

SEQ ID NO: 1 - protein, 7 aa, Artificial sequence
  synthetic sequence
CQCRRKN

SEQ ID NO: 2 - protein, 16 aa, Artificial sequence
  synthetic sequence
RRRRRRRRRCQCRRKN

SEQ ID NO: 3 - protein, 8 aa, Artificial sequence
  synthetic sequence
SIINFEKL

SEQ ID NO: 4 - protein, 9 aa, Artificial sequence
  synthetic sequence
SPSYVYHQF

SEQ ID NO: 5 - protein, 8 aa, Artificial sequence
  synthetic sequence
KSPWFTTL